(19) United States
(12) Patent Application Publication
(10) Pub. No.: US 2014/0243197 A1
(43) Pub. Date: Aug. 28, 2014

(54) CARBAMOYLMETHOXY- AND CARBAMOYLMETHYLTHIO- AND CARBAMOYLMETHYLAMINO BENZAMIDES FOR COMBATING INVERTEBRATE PESTS

(71) Applicants: Prashant Deshmukh, Mannheim (DE); Florian Kaiser, Mannheim (DE); Karsten Körber, Eppelheim (DE); Markus Kordes, Bobenheim-Roxheim (DE); Joachim Dickhaut, Heidelberg (DE); Arun Narine, Mannheim (DE); Nina Gertrud Bandur, Mannheim (DE); Gemma Veitch, Basel (CH); Deborah L. Culbertson, Fuquay Varina, NC (US); Paul Neese, Apex, NC (US); Koshi Gunjima, Cary, NC (US)

(72) Inventors: Prashant Deshmukh, Mannheim (DE); Florian Kaiser, Mannheim (DE); Karsten Körber, Eppelheim (DE); Markus Kordes, Bobenheim-Roxheim (DE); Joachim Dickhaut, Heidelberg (DE); Arun Narine, Mannheim (DE); Nina Gertrud Bandur, Mannheim (DE); Gemma Veitch, Basel (CH); Deborah L. Culbertson, Fuquay Varina, NC (US); Paul Neese, Apex, NC (US); Koshi Gunjima, Cary, NC (US)

(73) Assignee: BASF SE, Ludwigshafen (DE)

(21) Appl. No.: 14/239,221

(22) PCT Filed: Aug. 17, 2012
(86) PCT No.: PCT/EP2012/066140

§ 371 Date: Feb. 17, 2014

Related U.S. Application Data
(60) Provisional application No. 61/524,791, filed on Aug. 18, 2011.

(51) Int. Cl. C07D 401/04; A61K 31/4439; A01N 43/56

(52) U.S. Cl. CPC C07D 401/04; A01N 43/56; A61K 31/4439
USPC 504/100; 546/275.4; 514/341

(57) ABSTRACT

The present invention relates to substituted carbamoylmethoxy and carbamoylmethylthio benzamide compounds and the stereoisomers, salts, tautomers and N-oxides thereof and to compositions comprising the same. The invention also relates to the use of the carbamoylmethoxy- and carbamoylmethylthio-benzamide compounds or of the compositions comprising such compounds for combating invertebrate pests. Furthermore, the invention relates to methods of applying such compounds.

The present invention relates to substituted carbamoylmethoxy and carbamoylmethylthio benzamide compounds and the stereoisomers, salts, tautomers and N-oxides thereof and to compositions comprising the same. The invention also relates to the use of the carbamoylmethoxy- and carbamoylmethylthio-benzamide compounds or of the compositions comprising such compounds for combating invertebrate pests. Furthermore, the invention relates to methods of applying such compounds.

Invertebrate pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating invertebrate pests, in particular insects, arachnids and nematodes.

Certain substituted carbamoylmethoxy carbamoylmethylamino and carbamoylmethylthio benzamide compounds have been previously disclosed, for example in WO 2009/089680, WO 2005/054179, EP 133155, Bioorganic & Medicinal Chemistry Letters (2010), 20(2), 665-672 and Journal of Organic Chemistry (2001), 66(22), 7303-7312. Arylpyrazole anthranilic diamides and their use as insecticides have been presented in the IUPAC pesticides conference 2006 in Kobe, Japan. However, compounds with the characteristic substitution pattern as in the present invention have not yet been described.

It is therefore an object of the present invention to provide compounds that have a good pesticidal activity, in particular insecticidal activity, and show a broad activity spectrum against a large number of different invertebrate pests, especially against difficult to control arthropod pests and/or nematodes.

It has been found that these objectives can be achieved by carbamoylmethoxy- and carbamoylmethylthio- and carbamoylmethylamino benzamides compounds of the formula I below, by their stereoisomers and by their salts, in particular their agriculturally or veterinarily acceptable salts.

Therefore, in a first aspect, the invention relates to by carbamoylmethoxy- and carbamoylmethylthio- and carbamoylmethylamino benzamides compounds of compounds of formula (I)

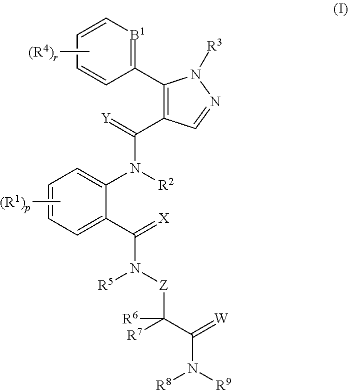

wherein
B¹ is N or CH;
each R¹ is independently selected from the group consisting of halogen; cyano; azido; nitro; —SCN; SF₅; C₁-C₆-alkyl which may be partially or fully halogenated and/or may be substituted by one or more radicals R¹⁰; C₃-C₈-cycloalkyl which may be partially or fully halogenated and/or may be substituted by one or more radicals R¹⁰; C₂-C₆-alkenyl which may be partially or fully halogenated and/or may be substituted by one or more radicals R¹⁰; C₂-C₆-alkynyl which may be partially or fully halogenated and/or may be substituted by one or more radicals R¹⁰; —Si(R¹⁴)₂R¹⁵; —OR¹¹; —OS(O)ₙR¹¹; —SR¹¹; —S(O)ₘR¹¹; —S(O)ₙN(R^{12a})R^{12b}; —N(R^{12a})R^{12b}; —N(R^{12a})C(═O)R¹⁰; —C(═O)R¹⁰; —C(═S)R¹⁰; —C(═O)OR¹¹; —C(═S)OR¹¹; —C(═NR^{12a})R¹⁰; —C(═O)N(R^{12a})R^{12b}; —C(═S)N(R^{12a})R^{12b}; phenyl which may be substituted by 1, 2, 3, 4 or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
   or two radicals R¹ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²¹—, —CH₂CH═N—, —CH═CH—NR²¹—, —OCH═N— and —SCH═N—, thus forming, together with the carbon atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R² is selected from the group consisting of hydrogen; cyano; C₁-C₁₀-alkyl which may be partially or fully halogenated and/or may be substituted by one or more radicals R¹⁰; C₃-C₈-cycloalkyl which may be partially or fully halogenated and/or may be substituted by one or more radicals R¹⁰; C₂-C₁₀-alkenyl which may be partially or fully halogenated and/or may be substituted by one or more radicals R¹⁰; C₂-C₁₀-alkynyl which may be partially or fully halogenated and/or may be substituted by one or more radicals R¹⁰; —N(R^{12a})R^{12b}; —Si(R¹⁴)₂R¹⁵; —OR¹¹; —SR¹¹; —S(O)ₘR¹¹; —S(O)ₙN(R^{12a})R^{12b}; —C(═O)R¹⁰; —C(═O)OR¹¹; —C(═O)N(R^{12a})R^{12b}; —C(═S)R¹⁰; —C(═S)OR¹¹; —C(═S)N(R^{12a})R^{12b}; —C(═NR^{12a})R¹⁰; phenyl which may be substituted by 1, 2, 3, 4 or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
R³ is selected from the group consisting of hydrogen, cyano, nitro, C₁-C₆-alkyl which may be partially or fully halogenated and/or may be substituted by one or more radicals R⁷, C₃-C₈-cycloalkyl which may be partially or fully halogenated and/or may be substituted by one or more radicals R⁷, C₂-C₆-alkenyl which may be partially or fully halogenated and/or may be substituted by one or more radicals R⁷, C₂-C₆-alkynyl which may be partially or fully halogenated and/or may be substituted by one or more radicals R⁷, —Si(R¹⁴)₂R¹³, —OR⁸, —OS(O)ₙR⁸, —SR⁸, —S(O)ₘR⁸, —S(O)ₙN(R^{9a})R^{9b}, —N(R^{9a})R^{9b}, N(R^{9a})C(═O)R⁷, —C(═O)R⁷, —C(═O)OR⁸, —C(═S)R⁷, —C(═S)OR⁸, —C(═NR^{9a})R⁷, —C(═O)N(R^{9a}) R^{9b}, —C(═S)N(R^{9a})R^{9b}, phenyl which may be substituted by 1, 2, 3, 4 or 5 radicals R¹⁰, and a 3-, 4-, 5-, 6- or 7-membered saturated, partially unsaturated or maximum unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R⁴ is independently selected from the group consisting of halogen, cyano, azido, nitro, —SCN, SF₅, C₁-C₆-alkyl which may be partially or fully halogenated and/or may be substituted by one or more radicals R¹⁰, C₃-C₈-cycloalkyl which may be partially or fully halogenated and/or may be substituted by one or more radicals R¹⁰, C₂-C₆-alkenyl which may be partially or fully halogenated and/or may be substituted by one or more radicals R¹⁰, C₂-C₆-alkynyl which may be partially or fully halogenated and/or may be substituted by one or more radicals R¹⁰, —Si(R¹⁴)₂R¹⁵, —OR¹¹, —SR¹¹, —S(O)ₘR¹¹, —S(O)ₙN(R^{12a})R^{12b}, —N(R^{12a})R^{12b}, —N(R^{12a})C(═O)R¹⁰, —C(═O)R¹⁰, —C(═O)OR¹¹, —C(═S)R¹⁰, —C(═S)OR¹¹, —C(═NR^{12a})R¹⁰, —C(═O)N(R^{12a})R^{12b}, —C(═S)N(R^{12a})R^{12b}, phenyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
R⁵ is selected from the group consisting of hydrogen; cyano; C₁-C₁₀-alkyl which may be partially or fully halogenated and/or may be substituted by one or more radicals R¹⁰; C₃-C₈-cycloalkyl which may be partially or fully halogenated and/or may be substituted by one or more radicals R¹⁰; C₂-C₁₀-alkenyl which may be partially or fully halogenated and/or may be substituted by one or more radicals R¹⁰; C₂-C₁₀-alkynyl which may be partially or fully halogenated and/or may be substituted by one or more radicals R¹⁰; —N(R^{12a})R^{12b}; —Si(R¹⁴)₂R¹⁵; —OR¹¹; —SR¹¹; —S(O)ₘR¹¹; —S(O)ₙN(R^{12a})R^{12b}; —C(═O)R¹⁰; —C(═O)OR¹¹; —C(═O)N(R^{12a})R^{12b}; —C(═S)R¹⁰; —C(═S)OR¹¹; —C(═S)N(R^{12a})R^{12b}; —C(═NR^{12a})R¹⁰; phenyl which may be substituted by 1, 2, 3, 4 or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
R⁶, R⁷ are selected independently of each other from the group consisting of hydrogen, halogen, cyano, azido, nitro, —SCN, C₁-C₆-alkyl, C₃-C₆-cycloalkyl, C₂-C₆-alkenyl, C₂-C₆-alkynyl, wherein the aforementioned aliphatic and cycloaliphatic radicals each independently may be partially or fully halogenated and/or may be substituted with 1, 2, 3, 4, 5, 6, 7, 8, 9 or 10 substituents R¹⁰, said substituents R¹⁰ being identical or different from one another if more than one substituent R¹⁰ is present, —OR¹¹, —NR^{12a}R^{12b}, —S(O)ₙR¹¹, —S(O)ₙNR^{12a}R^{12b}, —C(═O)R¹⁰, —C(═O)NR^{12a}R^{12b}, —C(═O)OR¹¹, —C(═S)R¹⁰, —C(═S)NR^{12a}R^{12b}, —C(═S)OR¹¹, —C(═S)SR¹¹, —C(═NR^{12a})R¹⁰, —C(═NR^{12a})NR^{12a}R^{12b}, —Si(R¹⁴)₂R¹⁵, phenyl which may be substituted with 1, 2, 3, 4 or 5 substituents R¹³, said substituents R¹³ being identical or different from one another if more than one substituent R¹³ is present, and a 3-, 4-, 5-, 6- or 7-membered saturated, partially unsaturated or maximally unsaturated heterocyclic ring wherein said heterocyclic ring comprises 1, 2 or 3 heteroatoms independently selected from the group consisting of oxygen, nitrogen and sulfur atoms and may be substituted with 1, 2, 3, 4 or 5 substituents R¹³, said substituents R¹³ being identical or different from one another if more than one substituent R¹³ is present, and wherein said nitrogen and sulfur atoms, independently of one another, may be oxidized;
   or R⁶ and R⁷ are together a C₂-C₇-alkylene or C₂-C₇-alkenylene chain and form a 3-, 4-, 5-, 6-, 7- or 8-membered saturated, partially unsaturated or maximally unsaturated ring together with the carbon atom they are bonded to, wherein 1, 2, 3 or 4 of any of the CH₂ groups in the C₂-C₇-alkylene chain or 1, 2, 3 or 4 of any of the CH₂ or CH groups in the C₂-C₇-alkenylene chain may be replaced by 1, 2, 3 or 4 groups independently selected from the group consisting of O, S, N and NH; and wherein the carbon and/or nitrogen atoms in the C₂-C₇-alkylene or C₂-C₇-alkenylene chain may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl and phenyl which may be substituted with 1, 2, 3, 4 or 5 substituents R¹³, said substituents R¹³ being identical or different from one another if more than one substituent R¹³ is present; and wherein the sulfur and nitrogen atoms in the C₂-C₇-alkylene, C₂-C₇-alkenylene or C₂-C₇-alkynylene chain, independently of one another, may be oxidized;
   or R⁶ and R⁷ together form a ═O, ═S, ═NR^{12a}, ═NOR¹¹, or ═CR¹⁶R¹⁷ radical;
R⁸, R⁹ are selected independently of each other from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals each independently may be partially or fully halogenated and/or may be substituted with 1, 2, 3, 4, 5, 6, 7, 8, 9 or 10 substituents R¹⁰, said substituents R¹⁰ being identical or different from one another if more than one substituent R¹⁰ is present, —OR¹¹, —NR^{12a}R^{12b}, —S(O)ₙR¹¹, —S(O)ₙNR^{12a}R^{12b}, —C(═O)R¹⁰, —C(═O)NR^{12a}R^{12b}, —C(═O)OR¹¹, —C(═S)R¹³, —C(═S)NR^{12a}R^{12b}, —C(═S)OR¹¹, —C(═S)SR¹¹, —C(═NR^{12a})R¹³, —C(═NR^{12a})NR^{12a}R^{12b}, —Si(R¹⁴)₂R¹⁵, phenyl which may be substituted with 1, 2, 3, 4 or 5 substituents R¹³ said substituents R¹³ being identical or different from one another if more than one substituent R¹³ is present, and a 3-, 4-, 5-, 6- or 7-membered saturated, partially unsaturated or maximum unsaturated heterocyclic ring wherein said heterocyclic ring comprises 1, 2 or 3 heteroatoms independently selected from the group consisting of oxygen, nitrogen and sulfur atoms and may be substituted with 1, 2, 3, 4 or 5 substituents R²¹, said substituents R²¹ being identical or different from one another if more than one substituent R²¹ is present, and wherein said nitrogen and sulfur atoms, independently of one another, may be oxidized;
   or R⁸ and R⁹, together with the nitrogen atom to which they are bound, form a 3-, 4-, 5-, 6-, 7- or 8-membered saturated, partially unsaturated or maximally unsaturated heterocyclic ring which may additionally contain 1, 2, 3 or 4 further heteroatoms or heteroatom groups selected from N, O, S, NO, SO, SO₂, C(═O) and C(═S) as ring members, where the heterocyclic ring may be substituted by one or more radicals R¹³;
   or R⁸ and R⁸ together may form a═CR¹⁶R¹⁷, ═S(O)ₙR¹¹, ═S(O)ₙNR^{12a}R^{12b}, ═NR^{12a} or ═NOR¹¹ radical;
each R¹⁰ is independently selected from the group consisting of cyano, azido, nitro, —SCN, SF₅, C₃-C₈-cycloalkyl, C₃-C₈-halocycloalkyl, —Si(R¹⁴)₂R¹⁵, —OR¹¹, —OSO₂R¹¹, —SR¹¹, —S(O)ₘR¹¹, —S(O)ₙN(R^{12a})R^{12b}, —N(R^{12a})R^{12b}, —C(═O)N(R^{12a})R^{12b}, —C(═S)N(R^{12a})R^{12b}, —C(═O)OR¹¹, —C(═O)R²⁰, phenyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
   and, in case R¹⁰ is bound to a cycloalkyl group or to a heterocyclic ring, R¹⁰ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹³;
   and in groups —C(═O)R¹⁰, —C(═S)R¹⁰, —C(═NR^{12a})R¹ and —N(R^{12a})C(═O)R¹⁰, R¹⁰ may additionally be selected from hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹³;
   or two geminally bound radicals R¹⁰ together form a group selected from ═CR¹⁶R¹⁷, ═S(O)ₘR¹¹, ═S(O)ₘN(R^{12a})R^{12b}, ═NR^{12a}, ═NOR¹¹ and ═NN(R^{12a})R^{12b};
   or two radicals R¹⁰, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R¹¹ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹⁵, —SR²³, —S(O)ₘR²³, —S(O)ₙN(R^{12a})R^{12b}, —N(R^{12a})R^{12b}, —N═CR¹⁸R¹⁹, —C(═O)R²⁰, —C(═O)N(R^{12a})R^{12b}, —C(═S)N(R^{12a})R^{12b}, —C(═O)OR²³, phenyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
   with the proviso that R¹¹ is not C₁-C₆-alkoxy or C₁-C₆-haloalkoxy if it is bound to an oxygen atom;
R^{12a}, R^{12b} are, independently of each other and independently of each occurrence, selected from the group consisting of hydrogen, cyano, C₁-C₆-alkyl which may be partially or fully halogenated and/or may be substituted by one or more radicals R²², C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R²², C₃-C₈-cycloalkyl which may be partially or fully halogenated and/or may be substituted by one or more radicals R²², C₃-C₈-cycloalkyl-C₁-C₄-alkyl where the cycloalkyl moiety may be partially or fully halogenated and/or may be substituted by one or more radicals R²², C₂-C₆-alkenyl which may be partially or fully halogenated and/or may be substituted by one or more radicals R²², C₂-C₆-alkynyl which may be partially or fully halogenated and/or may be substituted by one or more radicals R²², —N(R²⁴)R²⁵; —N(R²⁴)C(═O)R²⁰; —Si(R¹⁴)₂R¹⁵; —OR²³; —SR²³; —S(O)ₘR²³; —S(O)ₙN(R²⁴)R²⁵; —C(═O)R²⁰; —C(═O)OR²³; —C(═O)N(R²⁴)R²⁵; —C(═S)R²⁰; —C(═S)OR²³; —C(═S)N(R²⁴)R²⁵; —C(═NR²⁴)R²⁰; —S(O)ₘR²³, —S(O)ₙN(R²⁴)R²⁵; phenyl which may be substituted by 1, 2, 3, 4 or 5 radicals R¹³; benzyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
   or R^{12a} and R^{12b} together form a group ═CR¹⁶R¹⁷;
   or R^{12a} and R^{12b}, together with the nitrogen atom to which they are bound, may form a 3-, 4-, 5-, 6- or 7-membered saturated, partially unsaturated or maximally unsaturated heterocyclic ring which may additionally containing 1 or 2 further heteroatoms or heteroatom groups selected from N, O, S, NO, SO and SO₂, as ring members, where the heterocyclic ring may be substituted by one or more radicals R¹³;
each R¹³ is independently selected from the group consisting of halogen, cyano, azido, nitro, —SCN, SF₅, C₁-C₁₀-alkyl which may be partially or fully halogenated and/or may be substituted by one or more radicals R²², C₃-C₈-cycloalkyl which may be partially or fully halogenated and/or may be substituted by one or more radicals R²², C₂-C₁₀-alkenyl which may be partially or fully halogenated and/or may be substituted by one or more radicals R²², C₂-C₁₀-alkynyl which may be partially or fully halogenated and/or may be substituted by one or more radicals R²², —Si(R¹⁴)₂R¹⁵, —OR²³, —OS(O)ₙR²³, —SR²³, —S(O)ₘR²³, —S(O)ₙN(R²⁴)R²⁵, —N(R²⁴)R²⁵, C(═O)R²⁰, —C(═O)OR²³, —C(═NR²⁴)R²⁰, —C(═O)N(R²⁴)R²⁵, —C(═S)N(R²⁴)R²⁵, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;
   or two radicals R¹³ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²⁴—, —CH₂CH═N—, —CH═CH—NR²⁴—, —OCH═N— and —SCH═N—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R¹⁴, R¹⁵ are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;
R¹⁶, R¹⁷ are, independently of each other and independently of each occurrence, selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, —C(═O)R²⁰, —C(═O)OR²³, —C(═NR²⁴)R²⁰, —C(═O)N(R²⁴)R²⁵, —C(═S)N(R²⁴)R²⁵, phenyl which may be substituted by 1, 2, 3, 4, or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R¹³;
R¹⁸, R¹⁹ are, independently of each other and independently of each occurrence, selected from the group consisting of C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R¹³;
each R²⁰ is independently selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
each R²¹ is independently defined like R²;
each R²² is independently selected from the group consisting of cyano, azido, nitro, —SCN, SF₅, C₃-C₈-cycloalkyl, C₃-C₈-halocycloalkyl, —Si(R¹⁴)₂R¹⁵, —OR²³, —OSO₂R²³, —SR²³, —S(O)ₘR²³, —S(O)ₙN(R²⁴)R²⁵, —N(R²⁴)R²⁵, —C(═O)N(R²⁴)R²⁵, —C(═S)N(R²⁴)R²⁵, —C(═O)OR²³, —C(═O)R²³, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   and, in case R²² is bound to a cycloalkyl group, R²² may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl and C₂-C₆-haloalkynyl;
   or two geminally bound radicals R²² together form a group selected from ═S(O)ₘR²³, ═S(O)ₘN(R²⁴)R²⁵, ═NR²⁴, ═NOR²³ and ═NN(R²⁴)R²⁵;
   or two radicals R²², together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R²³ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹⁵, C₁-C₆-alkylaminosulfonyl, amino, C₁-C₆-alkylamino, di-(C₁-C₆-alkyl)-amino, C₁-C₆-alkylcarbonyl, C₁-C₆-haloalkylcarbonyl, aminocarbonyl, C₁-C₆-alkylaminocarbonyl, di-(C₁-C₆-alkyl)-aminocarbonyl, C₁-C₆-alkoxycarbonyl, C₁-C₆-haloalkoxycarbonyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   with the proviso that R²³ is not C₁-C₆-alkoxy or C₁-C₆-haloalkoxy if it is bound to an oxygen atom;
R²⁴ and R²⁵ are independently of each other and independently of each occurrence selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   or R²⁴ and R²⁵, together with the nitrogen atom to which they are bound, may form a 3-, 4-, 5-, 6- or 7-membered saturated, partially unsaturated or maximally unsaturated heterocyclic ring which may additionally contain 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy;
each m is independently 1 or 2;
each n is independently 0, 1 or 2;
p is 0, 1, 2, 3 or 4;
r is 0, 1, 2, 3, or 4;
W is O or S;
X is O or S;
Y is O or S; and
Z is O, NR^{12a} or S(O)ₙ
   or a stereoisomer, salt, tautomer or N-oxide thereof.

Furthermore, the invention relates to processes for the synthesis of compounds according to the invention and to intermediate compounds for the synthesis of compounds of formula (I).

The compounds of the present invention, i.e. the compounds of formula (I), their stereoisomers, their salts, their tautomers or their N-oxides, are particularly useful for controlling invertebrate pests, in particular for controlling arthropods and nematodes and especially insects. Therefore, the invention also relates to the use of a compound of the present invention, for combating or controlling invertebrate pests, in particular invertebrate pests of the group of insects, arachnids or nematodes.

The term “compound(s) according to the invention” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The term “composition(s) according to the invention” or “composition(s) of the present invention” comprises composition(s) comprising at least one compound according to the invention as defined above.

The invention also relates to a composition comprising at least one compound according to the invention, including a stereoisomer, salt, tautomer or N-oxide thereof, and at least one inert liquid and/or solid carrier. In particular, the invention relates to an agricultural or veterinary composition comprising at least one compound according to the invention including a stereoisomer, an agriculturally or veterinarily acceptable salt, tautomer or an N-oxide thereof, and at least one liquid and/or solid carrier.

The present invention also relates to a method for combating or controlling invertebrate pests, especially invertebrate pests of the group of insects, arachnids or nematodes, which method comprises contacting said pest or its food supply, habitat or breeding grounds with a pesticidally effective amount of at least one compound according to the invention including a stereoisomer, salt, tautomer or N-oxide thereof or a composition according to the invention.

The present invention also relates to a method for protecting growing plants from attack or infestation by invertebrate pests, especially invertebrate pests of the group of insects, arachnids or nematodes, which method comprises contacting a plant, or soil or water in which the plant is growing or may grow, with a pesticidally effective amount of at least one compound according to the invention including a stereoisomer, salt, tautomer or N-oxide thereof or a composition according to the invention.

The present invention also relates to a method for the protection of plant propagation material, preferably seeds, from soil insects and of the seedlings' roots and shoots from soil and foliar insects comprising contacting the seeds before sowing and/or after pregermination with at least one compound according to the invention including a stereoisomer, salt, tautomer or N-oxide thereof or a composition according to the invention.

The present invention also relates to plant propagation material, preferably seed, comprising a compound according to the invention including a stereoisomer, salt, tautomer or N-oxide thereof, preferably in an amount of from 0.01 g to 10 kg per 100 kg of the plant propagation material.

The present invention also relates to the use of a compound according to the invention including a stereoisomer, salt, tautomer or N-oxide thereof or a composition according to the invention for combating or controlling invertebrate pests of the group of insects, arachnids or nematodes.

The present invention also relates to the use of a compound according to the invention including a stereoisomer, salt or N-oxide thereof or a composition according to the invention for protecting growing plants from attack or infestation by invertebrate pests of the group of insects, arachnids or nematodes.

The present invention also relates to the use of a compound according to the invention including a stereoisomer, veterinarily acceptable salt, tautomer or N-oxide thereof or a composition according to the invention for combating or controlling invertebrate parasites in and on animals and to the use of a compound according to the invention including a stereoisomer, veterinarily acceptable salt, tautomer or N-oxide thereof or a composition according to the invention for preparing a medicament for combating or controlling invertebrate parasites in and on animals.

The present invention also relates to a method for treating an animal infested or infected by parasites or for preventing animals from getting infested or infected by parasites or for protecting an animal against infestation or infection by parasites which comprises orally, topically or parenterally administering or applying to the animal a parasiticidally effective amount of a compound according to the invention including a stereoisomer, veterinarily acceptable salt, tautomer or N-oxide thereof or a composition according to the invention.

The present invention also relates to the use of a compound according to the invention including a stereoisomer, veterinarily acceptable salt or N-oxide thereof or a composition according to the invention for the manufacture of a medicament for protecting an animal against infestation or infection by parasites or treating an animal infested or infected by parasites.

The present invention also relates to a process for the preparation of a composition for treating animals infested or infected by parasites, for preventing animals of getting infected or infested by parasites or protecting animals against infestation or infection by parasites which comprises a compound according to the invention including a stereoisomer, veterinarily acceptable salt, tautomer or N-oxide thereof.

The present invention also relates to a compound according to the invention including a stereoisomer, veterinarily acceptable salt, tautomer or N-oxide thereof for use as a medicament.

The present invention also relates to a compound according to the invention including a stereoisomer, veterinarily acceptable salt, tautomer or N-oxide thereof for use in the treatment, control, prevention or protection of animals against infestation or infection by parasites.

Depending on the substitution pattern, the compounds of the formula (I) may have one or more centers of chirality, in which case they are present as mixtures of enantiomers or diastereomers. The invention provides both the pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Another aspect may be the presence of atropisomerism due to hindered rotation of the amide moiety (for review articles on axial chirality and atropisomerism, see for example J. Clayden, Tetrahedron 2004, 60, 4335 and for the axial chirality arising from the sp²-sp² axis of the benzene-amide bond, see Y. Ishichi et al, Tetrahedron 2004, 60, 4481). Cis/trans isomers may be present with respect to an alkene, carbon-nitrogen double-bond, nitrogen-sulfur double bond or amide group. The term “stereoisomer(s)” encompasses both optical isomers, such as enantiomers or diastereomers, the latter existing due to more than one center of chirality in the molecule, as well as geometrical isomers (cis/trans isomers).

Depending on the substitution pattern, the compounds of the formula (I) may be present in the form of their tautomers. Hence the invention also relates to the tautomers of the formula (I) and the stereoisomers, salts, tautomers and N-oxides of said tautomers. For instance, if R⁴ is OH which is bound vicinally to B¹ and B¹ is N, or if R³ is H, the compounds (I) may be present in the below tautomeric forms (only two exemplary tautomer pairs are listed)

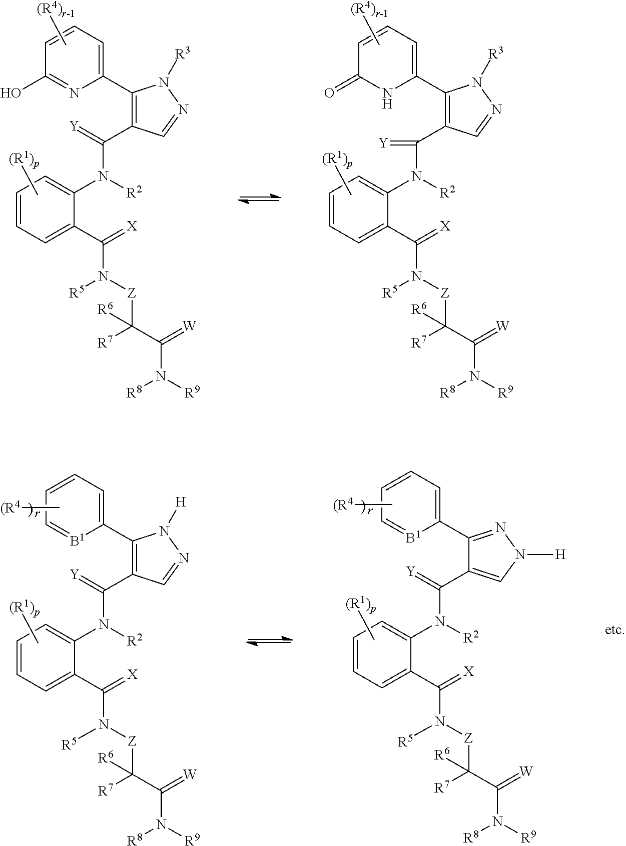

The term “N-oxide” includes any compound of the present invention which has at least one tertiary nitrogen atom that is oxidized to an N-oxide moiety. N-oxides are in particular possible in compounds (I) in which B¹ is N. N-oxides of such compounds can be prepared by oxidizing the ring nitrogen atom(s) with a suitable oxidizing agent, such as peroxo carboxylic acids or other peroxides.

The compounds of the present invention may be amorphous or may exist in one ore more different crystalline states (polymorphs) which may have different macroscopic properties such as stability or show different biological properties such as activities. The present invention includes both amorphous and crystalline compounds of formula (I), their enantiomers or diastereomers, mixtures of different crystalline states of the respective compound of formula (I), its enantiomers or diastereomers, as well as amorphous or crystalline salts thereof.

Salts of the compounds of the present invention are preferably agriculturally and veterinarily acceptable salts. They can be formed in a customary method, e.g. by reacting the compound with an acid if the compound of the present invention has a basic functionality or by reacting the compound with a suitable base if the compound of the present invention has an acidic functionality.

Suitable agriculturally acceptable salts are especially the salts of those cations or the acid addition salts of those acids whose cations and anions, respectively, do not have any adverse effect on the pesticidal action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds of the present invention with an acid of the corresponding anion, preferably of hydrochloric acid, hydrobromic acid, sulfuric acid, phosphoric acid or nitric acid.

Veterinarily acceptable salts of the compounds of the present invention encompass the salts of those cations or the acid addition salts which are known and accepted in the art for the formation of salts for veterinary use. Suitable acid addition salts, e.g. formed by compounds of the present invention containing a basic nitrogen atom, e.g. an amino group, include salts with inorganic acids, for example hydrochlorids, sulphates, phosphates, and nitrates and salts of organic acids for example acetic acid, maleic acid, e.g. the monoacid salts or diacid salts of maleic acid, dimaleic acid, fumaric acid, e.g. the monoacid salts or diacid salts of fumaric acid, difumaric acid, methane sulfenic acid, methane sulfonic acid, and succinic acid.

The term “invertebrate pest” as used herein encompasses animal populations, such as arthropod pests, including insects and arachnids, as well as nematodes, which may attack plants thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “plants” comprises any types of plants including “non-cultivated plants” and in particular “cultivated plants”.

The term “non-cultivated plants” refers to any wild type species or related species or related genera of a cultivated plant.

The term “cultivated plants” is to be understood as including plants which have been modified by breeding, mutagenesis or genetic engineering. Genetically modified plants are plants, the genetic material of which has been modified by the use of recombinant DNA techniques so that under natural circumstances it cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides), e.g. by glycosylation or polymer additions such as prenylated, acetylated or farnesylated moieties or PEG moieties (e.g. as disclosed in Biotechnol Prog. 2001 July-August; 17(4):720-8., Protein Eng Des Sel. 2004 January; 17(1):57-66, Nat Protoc. 2007; 2(5): 1225-35., Curr Opin Chem Biol. 2006 October; 10(5):487-91. Epub 2006 August 28., Biomaterials. 2001 March; 22(5):405-17, Bioconjug Chem. 2005 January-February; 16(1):113-21).

The term “cultivated plants” is to be understood also including plants that have been rendered tolerant to applications of specific classes of herbicides, such as hydroxyphenylpyruvate dioxygenase (HPPD) inhibitors; acetolactate synthase (ALS) inhibitors, such as sulfonyl ureas (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e. g. WO 92/00377); glutamine synthetase (GS) inhibitors, such as glufosinate (see e. g. EP-A-0242236, EP-A-242246) or oxynil herbicides (see e. g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e. g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as ä-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are disclosed, for example, in EP-A 374 753, WO 93/007278, WO 95/34656, EP-A 427 529, EP-A 451 878, WO 03/018810 und WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e. g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e. g. bio mass production, grain yield, starch content, oil content or protein content), tolerance to drought, salinity or other growth-limiting environ-mental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e. g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e. g. Amflora® potato).

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine. A partially or fully halogenated radical is termed below also “halo-radical”. For example, partially or fully halogenated alkyl is also termed haloalkyl.

The term “alkyl” as used herein (and in the alkyl moieties of other groups comprising an alkyl group, e.g. alkoxy, alkylcarbonyl, alkylthio, alkylsulfinyl, alkylsulfonyl and alkoxy-alkyl) denotes in each case a straight-chain or branched alkyl group having usually from 1 to 10 carbon atoms, frequently from 1 to 6 carbon atoms, preferably 1 to 4 carbon atoms and in particular from 1 to 3 carbon atoms. Examples of C₁-C₄-alkyl are methyl, ethyl, n-propyl, iso-propyl, n-butyl, 2-butyl (sec-butyl), isobutyl and tert-butyl. Examples for C₁-C₆-alkyl are, apart those mentioned for C₁-C₄-alkyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Examples for C₁-C₁₀-alkyl are, apart those mentioned for C₁-C₆-alkyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl, 2-propylpentyl, nonyl, decyl, 2-propylheptyl and 3-propylheptyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy, haloalkylthio, haloalkylcarbonyl, haloalkylsulfonyl and haloalkylsulfinyl) denotes in each case a straight-chain or branched alkyl group having usually from 1 to 10 carbon atoms (“C₁-C₁₀-haloalkyl”), frequently from 1 to 6 carbon atoms (“C₁-C₆-haloalkyl”), more frequently 1 to 4 carbon atoms (“C₁-C₄-haloalkyl”), wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl. Halomethyl is methyl in which 1, 2 or 3 of the hydrogen atoms are replaced by halogen atoms. Examples are bromomethyl, chloromethyl, dichloromethyl, trichloromethyl, fluoromethyl, difluoromethyl, trifluoromethyl, chlorofluoromethyl, dichlorofluoromethyl, chlorodifluoromethyl and the like. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl, pentafluoroethyl, and the like. Examples for C₁-C₂-haloalkyl are, apart those mentioned for C₁-C₂-fluoroalkyl, chloromethyl, dichloromethyl, trichloromethyl, bromomethyl, chlorofluoromethyl, dichlorofluoromethyl, chlorodifluoromethyl, 1-chloroethyl, 2-chloroethyl, 2,2,-dichloroethyl, 2,2,2-trichloroethyl, 2-chloro-2-fluoroethyl, 2-chloro-2,2-difluoroethyl, 2,2-dichloro-2-fluoroethyl, 1-bromoethyl, and the like. Examples for C₁-C₄-haloalkyl are, apart those mentioned for C₁-C₂-haloalkyl, 1-fluoropropyl, 2-fluoropropyl, 3-fluoropropyl, 3,3-difluoropropyl, 3,3,3-trifluoropropyl, heptafluoropropyl, 1,1,1-trifluoroprop-2-yl, 3-chloropropyl, 4-chlorobutyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms (“C₃-C₁₀-cycloalkyl”), preferably 3 to 8 carbon atoms (“C₃-C₈-cycloalkyl”) or in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl.

Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.1.1]hexyl, bicyclo[2.2.1]heptyl, bicyclo[3.1.1]heptyl, bicyclo[2.2.1]heptyl, bicyclo[2.2.2]octyl and bicyclo[3.2.1]octyl.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, preferably 3 to 8 carbon atoms or in particular 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “cycloalkyl-alkyl” used herein denotes a cycloalkyl group, as defined above, which is bound to the remainder of the molecule via an alkylene group. The term “C₃-C₈-cycloalkyl-C₁-C₄-alkyl” refers to a C₃-C₈-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “alkenyl” as used herein denotes in each case a monounsaturated straight-chain or branched hydrocarbon radical having usually 2 to 10 (“C₂-C₁₀-alkenyl”), preferably 2 to 6 carbon atoms (“C₂-C₆-alkenyl”), in particular 2 to 4 carbon atoms (“C₂-C₄-alkenyl”), and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) or 2 to 4 (“C₂-C₄-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “alkynyl” as used herein denotes unsaturated straight-chain or branched hydrocarbon radicals having usually 2 to 10 (“C₂-C₁₀-alkynyl”), frequently 2 to 6 (“C₂-C₆-alkynyl”), preferably 2 to 4 carbon atoms (“C₂-C₄-alkynyl”) and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like.

The term haloalkynyl as used herein, which is also expressed as “alkynyl which may be substituted by halogen”, refers to unsaturated straight-chain or branched hydrocarbon radicals having usually 3 to 10 carbon atoms (“C₂-C₁₀-haloalkynyl”), frequently 2 to 6 (“C₂-C₆-haloalkynyl”), preferably 2 to 4 carbon atoms (“C₂-C₄-haloalkynyl”), and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 10 carbon atoms (“C₁-C₁₀-alkoxy”), frequently from 1 to 6 carbon atoms (“C₁-C₆-alkoxy”), preferably 1 to 4 carbon atoms (“C₁-C₄-alkoxy”), which is bound to the remainder of the molecule via an oxygen atom. C₁-C₂-Alkoxy is methoxy or ethoxy. C₁-C₄-Alkoxy is additionally, for example, n-propoxy, 1-methylethoxy (isopropoxy), butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “haloalkoxy” as used herein denotes in each case a straight-chain or branched alkoxy group, as defined above, having from 1 to 10 carbon atoms (“C₁-C₁₀-haloalkoxy”), frequently from 1 to 6 carbon atoms (“C₁-C₆-haloalkoxy”), preferably 1 to 4 carbon atoms (“C₁-C₄-haloalkoxy”), more preferably 1 to 3 carbon atoms (“C₁-C₃-haloalkoxy”), wherein the hydrogen atoms of this group are partially or totally replaced with halogen atoms, in particular fluorine atoms. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

The term “alkoxy-alkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. “C₁-C₆-Alkoxy-C₁-C₆-alkyl” is a C₁-C₆-alkyl group, as defined above, in which one hydrogen atom is replaced by a C₁-C₆-alkoxy group, as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “haloalkoxy-alkyl” as used herein denotes in each case alkyl as defined above, usually comprising 1 to 6 carbon atoms, preferably 1 to 4 carbon atoms, wherein 1 carbon atom carries an haloalkoxy radical as defined above, usually comprising 1 to 10, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio “(also alkylsulfanyl or alkyl-S—)” as used herein denotes in each case a straight-chain or branched saturated alkyl group as defined above, usually comprising 1 to 10 carbon atoms (“C₁-C₁₀-alkylthio”), frequently comprising 1 to 6 carbon atoms (“C₁-C₆-alkylthio”), preferably 1 to 4 carbon atoms (“C₁-C₄-alkylthio”), which is attached via a sulfur atom at any position in the alkyl group. C₁-C₂-Alkylthio is methylthio or ethylthio. C₁-C₄-Alkylthio is additionally, for example, n-propylthio, 1-methylethylthio (isopropylthio), butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “haloalkylthio” as used herein refers to an alkylthio group as defined above wherein the hydrogen atoms are partially or fully substituted by fluorine, chlorine, bromine and/or iodine. C₁-C₂-Haloalkylthio is, for example, SCH₂F, SCHF₂, SCF₃, SCH₂Cl, SCHCl₂, SCC13, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₂-alkylsulfinyl” refers to a C₁-C₂-alkyl group, as defined above, attached via a sulfinyl [S(O)] group. The term “C₁-C₄-alkylsulfinyl” refers to a C₁-C₄-alkyl group, as defined above, attached via a sulfinyl [S(O)] group. The term “C₁-C₆-alkylsulfinyl” refers to a C₁-C₆-alkyl group, as defined above, attached via a sulfinyl [S(O)] group. C₁-C₂-alkylsulfinyl is methylsulfinyl or ethylsulfinyl. C₁-C₄-alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutyl-sulfinyl) or 1,1-dimethylethylsulfinyl (tert-butylsulfinyl). C₁-C₆-alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsuIfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The terms “haloalkylsulfinyl” and “S(O)ₙ-haloalkyl” (wherein n is 1) are equivalent and, as used herein, denote a haloalkyl group, as defined above, attached via a sulfinyl [S(O)] group. The term “S(O)ₙ—C₁-C₄-haloalkyl” (wherein n is 1), i.e. “C₁-C₄-haloalkylsulfinyl”, is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHC₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. The term “C₁-C₂-alkylsulfonyl” refers to a C₁-C₂-alkyl group, as defined above, attached via a sulfonyl [S(O)₂] group. The term “C₁-C₄-alkylsulfonyl” refers to a C₁-C₄-alkyl group, as defined above, attached via a sulfonyl [S(O)₂] group. The term “C₁-C₆-alkylsulfonyl” refers to a C₁-C₆-alkyl group, as defined above, attached via a sulfonyl [S(O)₂] group. C₁-C₂-alkylsulfonyl is methylsulfonyl or ethylsulfonyl. C₁-C₄-alkylsulfonyl is additionally, for example, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The terms “haloalkylsulfonyl” and “S(O)ₙ-haloalkyl” (wherein n is 2) are equivalent and, as used herein, denote a haloalkyl group, as defined above, attached via a sulfonyl [S(O)₂] group. The term “S(O)ₙ—C₁-C₄-haloalkyl” (wherein n is 2), i.e. “C₁-C₄-haloalkylsulfonyl”, is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₄-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonyl, 1-(CH₂Br)-2-bromoethylsulfonyl, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The term “alkylamino” as used herein denotes in each case a group —NHR, wherein R is a straight-chain or branched alkyl group usually having from 1 to 6 carbon atoms (“C₁-C₆-alkylamino”), preferably 1 to 4 carbon atoms (“C₁-C₄-alkylamino”). Examples of C₁-C₆-alkylamino are methylamino, ethylamino, n-propylamino, isopropylamino, n*butylamino, 2-butylamino, iso-butylamino, tert-butylamino, and the like.

The term “dialkylamino” as used herein denotes in each case a group-NRR′, wherein R and R′, independently of each other, are a straight-chain or branched alkyl group each usually having from 1 to 6 carbon atoms (“di-(C₁-C₆-alkyl)-amino”), preferably 1 to 4 carbon atoms (“di-(C₁-C₄-alkyl)-amino”). Examples of a di-(C₁-C₆-alkyl)-amino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The term “alkylaminosulfonyl” as used herein denotes in each case a straight-chain or branched alkylamino group as defined above, which is bound to the remainder of the molecule via a sulfonyl [S(O)₂] group. Examples of an alkylaminosulfonyl group are methylaminosulfonyl, ethylaminosulfonyl, n-propylaminosulfonyl, isopropylaminosulfonyl, n-butylaminosulfonyl, 2-butylaminosulfonyl, iso-butylaminosulfonyl, tertbutylaminosulfonyl, and the like.

The term “dialkylaminosulfonyl” as used herein denotes in each case a straight-chain or branched alkylamino group as defined above, which is bound to the remainder of the molecule via a sulfonyl [S(O)₂] group. Examples of a dialkylaminosulfonyl group are dimethylaminosulfonyl, diethylaminosulfonyl, dipropylaminosulfonyl, dibutylaminosulfonyl, methyl-ethyl-aminosulfonyl, methyl-propyl-aminosulfonyl, methylisopropylaminosulfonyl, methyl-butyl-aminosulfonyl, methyl-isobutyl-aminosulfonyl, ethyl-propyl-aminosulfonyl, ethyl-isopropylaminosulfonyl, ethyl-butyl-aminosulfonyl, ethyl-isobutyl-aminosulfonyl, and the like.

The suffix “-carbonyl” in a group denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “3- to 6-membered carbocyclic ring” as used herein refers to cyclopropane, cyclobutane, cyclopentane and cyclohexane rings.

The term “aryl” as used herein refers to a mono-, bi- or tricyclic aromatic hydrocarbon radical such as phenyl or naphthyl, in particular phenyl.

The term “het(ero)aryl” as used herein refers to a mono-, bi- or tricyclic heteroaromatic hydrocarbon radical, preferably to a monocyclic heteroaromatic radical, such as pyridyl, pyrimidyl and the like.

The term “3-, 4-, 5-, 6-, 7- or 8-membered saturated carbocyclic ring” as used herein refers to carbocyclic rings, which are monocyclic and fully saturated. Examples of such rings include cyclopropane, cyclobutane, cyclopentane, cyclohexane, cycloheptane, cyclooctane and the like.

The terms “3-, 4-, 5-, 6-, 7- or 8-membered partially unsaturated carbocyclic ring” and “5- or 6-membered partially unsaturated carbocyclic ring” refer to carbocyclic rings, which are monocyclic and have one or more degrees of unsaturation. Examples of such rings include cyclopropene, cyclobutene, cyclopentene, cyclohexene, cycloheptene, cyclooctene and the like.

The term “3-, 4-, 5-, 6- or 7-membered saturated, partially unsaturated or maximum unsaturated heterocyclic ring containing 1, 2 or 3 heteroatoms or heteroatom groups selected from N, O, S, NO, SO and SO₂, as ring members” [wherein “maximum unsaturated” includes also “aromatic”] as used herein denotes monocyclic radicals, the monocyclic radicals being saturated, partially unsaturated or maximum unsaturated (including aromatic). Unsaturated rings contain at least one C—C and/or C—N and/or N—N double bond(s). Maximum unsaturated rings contain as many conjugated C—C and/or C—N and/or N—N double bonds as allowed by the ring size. Maximum unsaturated 5- or 6-membered heterocyclic rings are aromatic. The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

Examples of a 3-, 4-, 5-, 6- or 7-membered saturated heterocyclic ring include: Oxiranyl, thiiranyl, aziridinyl, oxetanyl, thietanyl, azetidinyl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

A 3-, 4-, 5-, 6- or 7-membered maximum unsaturated (including aromatic) heterocyclic ring is e.g. a 5- or 6-membered maximum unsaturated (including aromatic) heterocyclic ring. Examples are: 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl.

The term “a 3-, 4-, 5-, 6- or 7-membered saturated, partially unsaturated or maximum unsaturated heterocyclic ring wherein said heterocyclic ring comprises 1, 2 or 3 heteroatoms independently selected from the group consisting of oxygen, nitrogen and sulfur atoms and wherein said nitrogen and sulfur atoms, independently of one another, may be oxidized”, is equivalent to the above-defined term “3-, 4-, 5-, 6- or 7-membered saturated, partially unsaturated or maximum unsaturated heterocyclic ring containing 1, 2 or 3 heteroatoms or heteroatom groups selected from N, O, S, NO, SO and SO₂, as ring members” [wherein “maximum unsaturated” includes also “aromatic”].

The term “a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members” as used herein denotes on the one side a “3-, 4-, 5-, 6-, 7- or 8-membered saturated carbocyclic ring” as defined above, with the exception of the maximum unsaturated ring systems, and on the other side “a saturated or partially unsaturated 3-, 4-, 5-, 6-, 7- or 8-membered heterocyclic ring containing 1, 2 or 3 heteroatoms or heteroatom groups selected from N, O, S, NO, SO and SO₂, as ring members”. The saturated or partially unsaturated 3-, 4-, 5-, 6- or 7-membered heterocyclic ring is as defined above.

When R^{12a} and R^{12b}, together with the nitrogen atom to which they are bound, or R²⁴ and R²⁵, together with the nitrogen atom to which they are bound, form a 3-, 4-, 5-, 6- or 7-membered saturated, partially unsaturated or maximum unsaturated heterocyclic ring which may additionally contain 1 or 2 further heteroatoms or heteroatom groups selected from N, O, S, NO, SO and SO₂, as ring members, this is an N-bound heterocyclic ring which apart the nitrogen ring atom may additionally contain 1, 2, 3 or 4 further heteroatoms or heteroatom groups selected from N, O, S, NO, SO and SO₂, as ring members. Examples are aziridin-1-yl, azetidin-1-yl, pyrrolidine-1-yl, pyrazolidin-1-yl, imidazolin-1-yl, oxazolidin-3-yl, isoxazolidin-3-yl, thiazolidin-1-yl, isothiazolidin-1-yl, triazolidin-1-yl, piperidin-1-yl, piperazin-1-yl, morpholin-4-yl, thiomorpholin-1-yl, 1,1-dioxothiomorpholin-4-yl, pyrrolin-1-yl, pyrrolin-1-yl, imidazolin-1-yl, dihydropyridin-1-yl, tetrahydropyridin-1-yl, pyrrol-1-yl, pyrazol-1-yl, imidazol-1-yl and the like.

When R⁸ and R⁹, together with the nitrogen atom to which they are bound, form a 3-, 4-, 5-, 6-, 7- or 8-membered saturated, partially unsaturated or maximally unsaturated heterocyclic ring which may additionally contain 1, 2, 3 or 4 further heteroatoms or heteroatom groups selected from N, O, S, NO, SO, SO₂, C(═O) and C(═S) as ring members, this is an N-bound heterocyclic ring which apart the nitrogen ring atom may additionally contain 1, 2, 3 or 4 further heteroatoms or heteroatom groups selected from N, O, S, NO, SO, SO₂, C(═O) and C(═S), as ring members. Examples are, apart those listed above for rings formed by R^{12a} and R^{12b} or R²⁴ and R²⁵ together with the nitrogen atom to which they are bound, pyrrolidine-2-one-1-yl, pyrrolidin-2,5-dione-1-yl, pyrrolidin-3-one-1-yl, pyrrolidin-2-thione-1-yl, pyrazolidin-3-one-1-yl, pyrazolidin-4-one-1-yl, imidazolidin-2-one-1-yl, imidazolidin-4-one-1-yl, piperidine-2-one-1-yl and the like.

The remarks made below as to preferred embodiments of the variables (substituents) of the compounds of formula (I) are valid on their own as well as preferably in combination with each other, as well as in combination with the stereoisomers, salts, tautomers or N-oxides thereof.

The remarks made below concerning preferred embodiments of the variables further are valid are valid on their own as well as preferably in combination with each other concerning the compounds of formula (I) as well as concerning the uses and methods according to the invention and the composition according to the invention.

As a matter of course, the r radicals R⁴ replace a hydrogen atom on a carbon ring atom. For instance, if B¹ is defined to be CH and if this position is to be substituted by a radical R⁴, then B¹ is of course C—R⁴. If there is more than one radical R⁴, these can be the same or different.

As a matter of course, the p radicals R¹ replace a hydrogen atom on a carbon ring atom. If there is more than one radical R¹, these can be the same or different.

A preferred compound according to the invention is a compound of formula (I) or a stereoisomer, salt, tautomer or N-oxide thereof, wherein the salt is an agriculturally or veterinarily acceptable salt. A further preferred compound according to the invention is a compound of formula (I) or a stereoisomer or salt thereof, especially an agriculturally or veterinarily acceptable salt. A most preferred compound according to the invention is a compound of formula (I) or a salt thereof, especially an agriculturally or veterinarily acceptable salt thereof.

Preferred is a compound of formula (I), wherein W is O.

Preferred is a compound of formula (I), wherein X is O.

Preferred is a compound of formula (I), wherein Y is O.

Preferred is a compound of formula (I), wherein W, X and Y are O.

Preferred is a compound of formula (I), wherein p is 1, 2 or 3, specifically 2.

Preferred is a compound of formula (I), wherein r is 0, 1, or 2, specifically 1.

Preferred is a compound of formula (I), wherein p is 1, 2 or 3, specifically 2; and r is 0, 1, or 2, specifically 1.

In a specific embodiment, B¹ is N.

Preferred is a compound of formula (I), wherein each R¹ is independently selected from halogen; cyano; C₁-C₆-alkyl which may be partially or fully halogenated and/or may be substituted by one or more, preferably 1, 2 or 3, more preferably 1 or 2, in particular 1, radicals R¹⁰; C₃-C₈-cycloalkyl which may be partially or fully halogenated and/or may be substituted by one or more, preferably 1, 2 or 3, more preferably 1 or 2, in particular 1, radicals R¹⁰; C₂-C₆-alkenyl which may be partially or fully halogenated and/or may be substituted by one or more, preferably 1, 2 or 3, more preferably 1 or 2, in particular 1, radicals R¹⁰; C₂-C₆-alkynyl which may be partially or fully halogenated and/or may be substituted by one or more, preferably 1, 2 or 3, more preferably 1 or 2, in particular 1, radicals R¹⁰; —OR¹¹; —OS(O)ₙR¹¹; —SR¹¹; —S(O)ₘR¹¹; —S(O)ₙN(R^{12a})R^{12b}; —N(R^{12a})R^{12b}; —N(R^{12a})C(═O)R¹⁰; C(═O)R¹⁰; —C(═O)OR¹¹; phenyl which may be substituted by 1, 2 or 3 radicals R¹³; and a 5- or 6-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2, in particular 1, radicals R¹³; or two radicals R¹ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²¹—, —CH₂CH═N—, —CH═CH—NR²¹—, —OCH═N— and —SCH═N—, thus forming, together with the carbon atoms to which they are bound, a 5- or 6-membered ring, where the hydrogen atoms of the above groups may be replaced by one or more, preferably 1, 2 or 3, more preferably 1 or 2, in particular 1, substituents selected from halogen, methyl, halomethyl, hydroxyl, methoxy and halomethoxy or one or more, preferably 1 or 2, CH₂ groups of the above groups may be replaced by a C═O group.

In particular, each R¹ is independently selected from halogen, cyano and C₁-C₆-alkyl which may be partially or fully halogenated and/or may be substituted by one or more, preferably 1, 2 or 3, more preferably 1 or 2, in particular 1, radicals R¹⁰. More particularly, each R¹ is independently selected from halogen, cyano, C₁-C₆-alkyl and C₁-C₄-haloalkyl. Specifically, each R¹ is independently selected from halogen, C₁-C₄-alkyl and CF₃, more specifically from halogen and C₁-C₄-alkyl, and very specifically from Cl and methyl.

Preferred is a compound of formula (I), wherein R² is hydrogen or C₁-C₆-alkyl which may be partially or fully halogenated and/or may be substituted by one or more, preferably 1, 2 or 3, more preferably 1 or 2, in particular 1, radicals R¹⁰. More preferably, R² is hydrogen or C₁-C₄-alkyl. Specifically R² is hydrogen.

Preferred is a compound of formula (I), wherein R³ is selected from hydrogen; cyano; C₁-C₆-alkyl which may be partially or fully halogenated and/or may be substituted by one or more, preferably 1, 2 or 3, more preferably 1 or 2, in particular 1, radicals R⁷; C₃-C₈-cycloalkyl which may be partially or fully halogenated and/or may be substituted by one or more, preferably 1, 2 or 3, more preferably 1 or 2, in particular 1, radicals R⁷; C₂-C₆-alkenyl which may be partially or fully halogenated and/or may be substituted by one or more, preferably 1, 2 or 3, more preferably 1 or 2, in particular 1, radicals R⁷; C₂-C₆-alkynyl which may be partially or fully halogenated and/or may be substituted by one or more, preferably 1, 2 or 3, more preferably 1 or 2, in particular 1, radicals R⁷; C(═O)R⁷; —C(═O)OR⁸; phenyl which may be substituted by 1, 2 or 3 radicals R¹⁰; and a 5- or 6-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2, in particular 1, radicals R¹⁰. More preferably, R³ is selected from hydrogen, cyano and C₁-C₆-alkyl which may be partially or fully halogenated and/or may be substituted by one or more, preferably 1, 2 or 3, more preferably 1 or 2, in particular 1, radicals R⁷. In particular, R³ is selected from hydrogen and C₁-C₆-alkyl which may be partially or fully halogenated and/or may be substituted by one or more, preferably 1, 2 or 3, more preferably 1 or 2, in particular 1, radicals R⁷. More particularly, R³ is selected from C₁-C₆-alkyl and C₁-C₄-haloalkyl. Specifically, R³ is selected from C₁-C₆-alkyl and C₁-C₄-haloalkyl, more specifically from C₁-C₄-alkyl and fluorinated C₁-C₄-alkyl, and very specifically from C₁-C₄-alkyl and fluorinated C₁-C₂-alkyl.

In a specific embodiment, R³ has one of the general or preferred meanings given above, except for hydrogen.

Preferred is a compound of formula (I), wherein each R⁴ is independently selected from halogen; cyano; C₁-C₆-alkyl which may be partially or fully halogenated and/or may be substituted by one or more, preferably 1, 2 or 3, more preferably 1 or 2, in particular 1, radicals R¹⁰; C₃-C₈-cycloalkyl which may be partially or fully halogenated and/or may be substituted by one or more, preferably 1, 2 or 3, more preferably 1 or 2, in particular 1, radicals R¹⁰; C₂-C₆-alkenyl which may be partially or fully halogenated and/or may be substituted by one or more, preferably 1, 2 or 3, more preferably 1 or 2, in particular 1, radicals R¹⁰; C₂-C₆-alkynyl which may be partially or fully halogenated and/or may be substituted by one or more, preferably 1, 2 or 3, more preferably 1 or 2, in particular 1, radicals R¹⁰; —OR¹¹; —OS(O)ₙR¹¹; —SR¹¹; —S(O)ₘR¹¹; —S(O)ₙN(R^{12a})R^{12b}; —N(R^{12a})R^{12b}; —N(R^{12a})C(═O)R¹⁰; C(═O)R¹⁰; —C(═O)OR¹¹; phenyl which may be substituted by 1, 2 or 3 radicals R¹³; and a 5- or 6-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2, in particular 1, radicals R¹³. In particular, each R⁴ is independently selected from halogen, cyano and C₁-C₆-alkyl which may be partially or fully halogenated and/or may be substituted by one or more, preferably 1, 2 or 3, more preferably 1 or 2, in particular 1, radicals R¹⁰. More particularly, each R⁴ is independently selected from halogen and C₁-C₄-haloalkyl. Specifically, each R⁴ is independently selected from halogen and very specifically from Cl.

Preferred is a compound of formula (I), wherein R⁵ is hydrogen or C₁-C₆-alkyl which may be partially or fully halogenated and/or may be substituted by one or more, preferably 1, 2 or 3, more preferably 1 or 2, in particular 1, radicals R¹⁰. More preferably, R⁵ is hydrogen or C₁-C₄-alkyl. Specifically R⁵ is hydrogen.

Preferred is a compound of formula (I), wherein R⁶ and R⁷, independently of each other, are selected from hydrogen, halogen and C₁-C₆-alkyl which may be partially or fully halogenated and/or may be substituted by one or more radicals R¹⁰. More preferably, R⁶ and R⁷, independently of each other, are selected from hydrogen, halogen, C₁-C₆-alkyl, and C₁-C₆-haloalkyl. Specifically, R⁶ and R⁷ are hydrogen.

Preferred is a compound of formula (I), wherein R⁸ and R⁹ are independently selected from hydrogen, C₁-C₆-alkyl which may be partially or fully halogenated and/or may be substituted by one or more, preferably 1, 2 or 3, more preferably 1 or 2, in particular 1, radicals R¹⁰, C₂-C₆-alkenyl which may be partially or fully halogenated and/or may be substituted by one or more, preferably 1, 2 or 3, more preferably 1 or 2, in particular 1, radicals R¹⁰, C₂-C₆-alkynyl which may be partially or fully halogenated and/or may be substituted by one or more, preferably 1, 2 or 3, more preferably 1 or 2, in particular 1, radicals R¹⁰, C₃-C₆-cycloalkyl which may be partially or fully halogenated and/or may be substituted by one or more, preferably 1, 2 or 3, more preferably 1 or 2, in particular 1, radicals R¹⁰, phenyl which may be substituted by 1, 2, 3, 4 or 5 radicals R¹³, a 3-, 4-, 5-, 6- or 7-membered saturated, partially unsaturated or maximum unsaturated heterocyclic ring containing 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2, in particular 1, radicals R¹³, —C(═O)R¹⁰, —C(═O)OR¹¹ and —C(═O)N(R^{12a})R^{12b}; or R⁸ and R⁹, together with the nitrogen atom to which they are attached, form a saturated, partially unsaturated or aromatic 5- or 6-membered ring which optionally contains 1 or 2 further heteroatoms or heteroatom groups selected from N, O, S, NO, SO and SO₂, as ring members, and which may be substituted by 1, 2 or 3 radicals R¹³.

More preferably, R⁸ and R⁹, independently of each other, are selected from the group consisting of hydrogen, C₁-C₆-alkyl which may be partially or fully halogenated and/or may be substituted by one or more, preferably 1, 2 or 3, more preferably 1 or 2, in particular 1, radicals R¹⁰, —C(═O)R¹⁰, —C(═O)OR¹¹, and —C(═O)N(R^{12a})R^{12b}, or, together with the nitrogen atom to which they are bound, form a 5- or 6-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals selected from halogen, cyano, C₁-C₆-alkyl, C₁-C₆-haloalkyl, C₁-C₆-alkoxy and C₁-C₆-haloalkoxy.

In particular, R⁸ and R⁹, independently of each other, are selected from the group consisting of hydrogen, C₁-C₆-alkyl, C₁-C₆-haloalkyl and C₁-C₆-alkyl substituted by phenyl which may carry 1, 2 or 3 substituents R¹³. Specifically, R⁸ and R⁹, independently of each other, are selected from the group consisting of hydrogen and C₁-C₆-alkyl. More specifically one of R⁸ and R⁹ is hydrogen or methyl and the other is C₁-C₆-alkyl.

In one preferred embodiment, Z is O. In another preferred embodiment, Z is NR^{12a}.

If Z is NR^{12a}, R^{12a} is preferably hydrogen or C₁-C₆-alkyl which may be partially or fully halogenated and/or may be substituted by one or more radicals R²², and is specifically hydrogen.

In case R¹⁰ is a substituent on an alkyl, alkenyl or alkynyl group, it is preferably selected from the group consisting of cyano, azido, nitro, —SCN, SF₅, C₃-C₈-cycloalkyl, C₃-C₈-halocycloalkyl, —Si(R¹⁴)₂R¹⁵, —OR¹¹, —OSO₂R¹¹, —SR¹¹, —S(O)ₘR¹¹, —S(O)ₙN(R^{12a})R^{12b}, —N(R^{12a})R^{12b}, —C(═O)N(R^{12a})R^{12b}, —C(═S)N(R^{12a})R^{12b}, —C(═O)OR¹¹, —C(═O)R²⁰, phenyl which may be substituted by 1, 2, 3, 4 or 5 radicals R¹³, and a 3-, 4-, 5-, 6- or 7-membered saturated, partially unsaturated or maximum unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³; or two geminally bound radicals R¹⁰ together form a group selected from ═CR¹⁶R¹⁷, ═S(O)ₘR¹¹, ═S(O)ₘN(R^{12a})R^{12b}, ═NR^{12a}, ═NOR¹¹ and ═NNR^{12a}R^{12b}; or two radicals R¹⁰, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members, where R¹¹, R^{12a}, R^{12b}, R¹³, R¹⁴, R¹⁵, R¹⁶, R¹⁷ and R²⁰ have one of the meanings given above or in particular one of the preferred meanings given below.

In case R¹⁰ is a substituent on an alkyl, alkenyl or alkynyl group, it is more preferably selected from the group consisting of cyano, C₃-C₈-cycloalkyl, C₃-C₈-halocycloalkyl, —OR¹¹, —SR¹¹, —C(═O)N(R^{12a})R^{12b}, —C(═S)N(R^{12a})R^{12b}, —C(═O)OR¹¹, —C(═O)R²⁰, —C(═NR^{12a})R²⁰, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹¹, R^{12a}, R^{12b}, R¹³ and R²⁰ have one of the meanings given above or in particular one of the preferred meanings given below.

In case R¹⁰ is a substituent on an alkyl, alkenyl or alkynyl group, it is even more preferably selected from the group consisting of cyano, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, —C(═O)N(R^{12a})R^{12b}, —C(═S)N(R^{12a})R^{12b}, —C(═O)OR¹¹, —C(═O)R²⁰, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹¹, R^{12a}, R^{12b}, R¹³, and R²⁰ have one of the meanings given above or in particular one of the preferred meanings given below.

In case R²⁰ is a substituent on an alkyl, alkenyl or alkynyl group, it is in particular selected from the group consisting of cyano, C₃-C₆-cycloalkyl, C₃-C₆-halocycloalkyl, —C(═O)N(R^{12a})R^{12b}, —C(═S)N(R^{12a})R^{12b}, —C(═O)OR¹¹, —C(═O)R²⁰, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹¹, R^{12a}, R^{12b}, R¹³ and R²⁰ have one of the meanings given above or in particular one of the preferred meanings given below.

In case R¹⁰ is a substituent on a cycloalkyl group, it is preferably selected from the group consisting of cyano, azido, nitro, —SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹⁵, —OR¹¹, —OSO₂R¹¹, —SR¹¹, —S(O)ₘR¹¹, —S(O)ₙN(R^{12a})R^{12b}, —N(R^{12a})R^{12b}, —C(═O)N(R^{12a})R^{12b}, —C(═S)N(R^{12a})R^{12b}, —C(═O)OR¹¹, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³;

or two geminally bound radicals R⁷ together form a group selected from ═CR¹⁶R¹⁷, ═S(O)ₘR¹¹, ═S(O)ₘN(R^{12a})R^{12b}, ═NR^{12a}, ═NOR¹¹ and ═NNR^{12a}R^{12b};
or two radicals R¹⁰, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members,
where R¹¹, R^{12a}, R^{12b}, R¹³, R¹⁴, R¹⁵, R¹⁶, R¹⁷ and R²⁰ have one of the meanings given above or in particular one of the preferred meanings given below.

In case R¹⁰ is a substituent on a cycloalkyl group, it is more preferably selected from the group consisting of halogen, cyano, C₁-C₆-alkyl, C₁-C₆-haloalkyl, C₁-C₆-alkoxy-C₁-C₆-alkyl, —OR¹¹, —OSO₂R¹¹, —SR¹¹, —S(O)ₘR¹¹, —S(O)ₙN(R^{12a})R^{12b}, —N(R^{12a})R^{12b}, —C(═O)N(R^{12a})R^{12b}, —C(═S)N(R^{12a})R^{12b}, —C(═O)OR¹¹, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹¹, R^{12a}, R^{12b} and R¹³ have one of the meanings given above or in particular one of the preferred meanings given below.

In case R¹⁰ is a substituent on a cycloalkyl group, it is even more preferably selected from the group consisting of halogen, C₁-C₄-alkyl, C₁-C₃-haloalkyl, C₁-C₄-alkoxy and C₁-C₃-haloalkoxy. In particular, R¹⁰ as a substituent on a cycloalkyl group is selected from halogen, C₁-C₄-alkyl and C₁-C₃-haloalkyl.

In case R¹⁰ is a substituent on C(═O), C(═S) or C(═NR^{12a}), it is preferably selected from the group consisting of hydrogen, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —OR¹¹, —SR¹¹, —N(R^{12a})R^{12b}, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹¹, R^{12a}, R^{12b} and R¹³ have one of the meanings given above or in particular one of the preferred meanings given below.

In case R¹⁰ is a substituent on C(═O), C(═S) or C(═NR^{12a}), it is more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹³ has one of the meanings given above or in particular one of the preferred meanings given below.

In case R¹⁰ is a substituent on C(═O), C(═S) or C(═NR^{12a}), it is more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl which may be substituted by 1, 2, 3, 4 or 5 radicals R¹³, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹³ has one of the meanings given above or in particular one of the preferred meanings given below.

In case R¹⁰ is a substituent on C(═O), C(═S) or C(═NR^{12a}), it is even more preferably selected from the group consisting of C₁-C₄-alkyl, C₁-C₃-haloalkyl, C₃-C₆-cycloalkyl, C₃-C₆-halocycloalkyl, C₁-C₄-alkoxy, C₁-C₃-haloalkoxy, phenyl which may be substituted by 1, 2, 3, 4 or 5 radicals R¹³, and a 5- or 6-membered heteroaromatic ring containing 1, 2 or 3 heteroatoms selected from N, O and S, as ring members, where the heteroaromatic ring may be substituted by one or more radicals R¹³; where R¹³ has one of the meanings given above or in particular one of the preferred meanings given below.

Preferably, each R¹¹ is independently selected from the group consisting of hydrogen, C₁-C₆-alkyl, C₁-C₆-haloalkyl, C₃-C₈-cycloalkyl, C₃-C₈-halocycloalkyl, C₃-C₈-cycloalkyl-C₁-C₄-alkyl, phenyl which may be substituted by 1, 2, 3, 4 or 5 radicals R¹³; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹³, where R¹³ has one of the meanings given above or in particular one of the preferred meanings given below.

More preferably, each R¹¹ is independently selected from the group consisting of hydrogen, C₁-C₆-alkyl, C₁-C₆-haloalkyl, phenyl which may be substituted by 1, 2, 3, 4 or 5 radicals R¹³; and a 5- or 6-membered heteroaromatic ring containing 1, 2 or 3 heteroatoms selected from N, O and S, as ring members, where the heteroaromatic ring may be substituted by one or more radicals R¹³; where R¹³ has one of the meanings given above or in particular one of the preferred meanings given below.

R^{12a} and R^{12b} are independently of each other and independently of each occurrence preferably selected from the group consisting of hydrogen, cyano, C₁-C₆-alkyl which may be partially or fully halogenated and/or may be substituted by one or more radicals R²², C₂-C₆-alkenyl which may be partially or fully halogenated and/or may be substituted by one or more radicals R²², C₂-C₆-alkynyl which may be partially or fully halogenated and/or may be substituted by one or more radicals R²², C₃-C₈-cycloalkyl, C₃-C₈-halocycloalkyl, C₃-C₈-cycloalkyl-C₁-C₆-alkyl, S(O)ₘR²³, S(O)ₙNR²⁴R²⁵, phenyl which may be substituted by 1, 2, 3, 4 or 5 radicals R¹³, benzyl wherein the phenyl moiety may be substituted by 1, 2, 3, 4 or 5 radicals R¹³, and a 5- or 6-membered heterocyclic ring containing 1, 2 or 3 heteroatoms or heteroatom groups selected from N, O, S, NO, SO and SO₂, as ring members, where the heterocyclic ring may be substituted by one or more radicals R¹³; where R¹³, R²³, R²⁴ and R²⁵ have one of the meanings given above or in particular one of the preferred meanings given below; or R^{12a} and R^{12b} together form a group ═CR¹⁶R¹⁷, where R¹⁶ and R¹⁷ have one of the meanings given above or in particular one of the preferred meanings given below; or R^{12a} and R^{12b}, together with the nitrogen atom to which they are bound, form a 3-, 4-, 5-, 6- or 7-membered saturated, partially unsaturated or aromatic, preferably a saturated, heterocyclic ring which may additionally containing 1 or 2 further heteroatoms or heteroatom groups selected from N, O, S, NO, SO and SO₂, as ring members, where the heterocyclic ring may be substituted by one or more radicals R¹³, where R¹³ has one of the meanings given above or in particular one of the preferred meanings given below.

In the above preferred embodiment of R^{12a} and R^{12b}, R¹⁶ is preferably hydrogen or methyl and R¹⁷ is preferably C₁-C₆-alkoxy, C₁-C₆-haloalkoxy, —C(═O)R²⁰, —C(═O)OR²³, or —C(═O)N(R²⁴)R²⁵, where R²⁰, R²³, R²⁴ and R²⁵ have one of the meanings given above or in particular one of the preferred meanings given below.

In the above preferred embodiment of R^{12a} and R^{12b}, R^{12a} and R^{12b}, if they do not form together a group ═CR¹⁶R¹⁷ or together with the N atom to which they are bound a heterocyclic ring, are preferably selected from hydrogen, cyano, C₁-C₄-alkyl, C₁-C₄-haloalkyl, cyclopropyl, C₁-C₄-alkylcarbonyl, C₁-C₄-haloalkylcarbonyl, C₁-C₄-alkoxycarbonyl and C₁-C₄-haloalkoxycarbonyl and are more preferably hydrogen or C₁-C₄-alkyl.

If R^{12a} and R^{12b}, together with the nitrogen atom to which they are bound, form a 3-, 4-, 5-, 6- or 7-membered saturated, partially unsaturated or aromatic heterocyclic ring which may additionally contain 1 or 2 further heteroatoms or heteroatom groups selected from N, O, S, NO, SO and SO₂, as ring members, this is preferably a 3, 5 or 6-membered saturated heterocyclic ring which may additionally contain 1 further heteroatom or heteroatom group selected from N, O, S, NO, SO and SO₂, as ring member.

Specifically, R^{12a} and R^{12b} are independently of each other and independently of each occurrence selected from the group consisting of hydrogen, C₁-C₆-alkyl, C₁-C₆-haloalkyl, C₂-C₆-alkynyl, C₃-C₈-cycloalkyl-C₁-C₆-alkyl, benzyl wherein the phenyl moiety may be substituted by 1, 2, 3, 4 or 5 radicals R¹³, and a 5- or 6-membered heterocyclic ring containing 1, 2 or 3 heteroatoms or heteroatom groups selected from N, O, S, NO, SO and SO₂, as ring members, where the heterocyclic ring may be substituted by one or more radicals R¹³. More specifically, R^{12b} is hydrogen or C₁-C₄-alkyl and R^{12a} has one of the meanings specified above.

Preferably, each R¹³ is independently selected from the group consisting of halogen, cyano, C₁-C₁₀-alkyl which may be partially or fully halogenated and/or may be substituted by one or more radicals R²², C₃-C₈-cycloalkyl which may be partially or fully halogenated and/or may be substituted by one or more radicals R²², —OR²³, —OS(O)ₙR²³, —SR²³, —S(O)ₘR²³, —S(O)ₙN(R²⁴)R²⁵, —N(R²⁴)R²⁵, C(═O)R²⁰, —C(═O)OR²³, —C(═O)N(R²⁴)R²⁵, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or maximum unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy; or two radicals R¹³ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, and —O(CH₂)O—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group,

where R²⁰, R²³, R²⁴ and R²⁵ have one of the general or in particular one of the preferred meanings given above.

More preferably, each R¹³ is independently selected from the group consisting of halogen, cyano, C₁-C₁₀-alkyl which may be partially or fully halogenated and/or may be substituted by one or more radicals R²², —OR²³, —N(R²⁴)R²⁵, C(═O)R²⁰, —C(═O)OR²³, —C(═O)N(R²⁴)R²⁵, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy; where R²⁰, R²³, R²⁴ and R²⁵ have one of the general or in particular one of the preferred meanings given above.

Even more preferably, each R¹³ is independently selected from the group consisting of halogen, C₁-C₄-alkyl, C₁-C₄-haloalkyl, C₁-C₄-alkoxy and C₁-C₄-haloalkoxy. In particular, each R¹³ is independently selected from the group consisting of halogen, C₁-C₄-alkyl and C₁-C₄-haloalkyl.

Preferably, R¹⁴ and R¹⁵ are, independently of each other and independently of each occurrence, selected from C₁-C₄-alkyl and are in particular methyl.

Preferably, R¹⁶ and R¹⁷ are, independently of each other and independently of each occurrence, selected from the group consisting of hydrogen, halogen, C₁-C₆-alkyl and C₁-C₆-haloalkyl. More preferably, R¹⁶ and R¹⁷ are, independently of each other and independently of each occurrence, selected from the group consisting of hydrogen, halogen and C₁-C₆-alkyl and in particular from the group consisting of hydrogen and halogen. Specifically, they are hydrogen.

Preferably, R¹⁸ and R¹⁹ are, independently of each other and independently of each occurrence, selected from the group consisting of C₁-C₆-alkyl, C₁-C₆-haloalkyl and phenyl which may be substituted by 1, 2, 3, 4, or 5 radicals R¹³; where R¹³ has one of the general or in particular one of the preferred meanings given above.

Preferably, each R²⁰ is independently selected from the group consisting of C₁-C₆-alkyl, C₁-C₆-haloalkyl, C₃-C₈-cycloalkyl, C₃-C₈-halocycloalkyl, phenyl and benzyl. More preferably, each R²⁰ is independently selected from the group consisting of C₁-C₆-alkyl, C₁-C₆-haloalkyl and phenyl and is in particular C₁-C₄-alkyl or C₁-C₃-haloalkyl.

Preferably, each R²¹ is independently selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R¹⁰; —C(═O)R¹⁰; —C(═O)OR¹¹; —C(═O)N(R^{12a})R^{12b}; —C(═S)R¹⁰; —C(═S)OR¹¹; —C(═S)N(R^{12a})R^{12b} and —C(═NR^{12a})R¹⁰; where R¹⁰, R¹¹, R^{12a} and R^{12b} have one of the general or in particular one of the preferred meanings given above.

More preferably, each R²¹ is selected from the group consisting of hydrogen; C₁-C₆-alkyl which may be partially or fully halogenated and/or may be substituted by one or more, e.g. 1, 2, 3 or 4, preferably 1 or 2, more preferably 1, radicals R¹⁰; —C(═O)R¹⁰ and —C(═O)N(R^{12a})R^{12b}; where R¹⁰, R^{12a} and R^{12b} have one of the general or in particular one of the preferred meanings given above. Preferably, in this case, R¹⁰ as a C₁-C₆-alkyl substituent, is selected from CN, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, phenyl and a 5- or 6-membered hetaryl ring containing 1, 2 or 3 heteroatoms selected from N, O and S as ring members and being optionally substituted by 1, 2 or 3 radicals R¹³. In this case, R¹⁰ as a CO substituent, is preferably selected from C₁-C₆-alkyl, C₁-C₆-haloalkyl, C₁-C₆-alkoxy and C₁-C₆-haloalkoxy. In this case, R^{12a} and R^{12b} are preferably selected from hydrogen and C₁-C₆-alkyl.

In particular, each R²¹ is selected from the group consisting of hydrogen, C₁-C₄-alkyl, C₁-C₄-haloalkyl and —C(═O)R¹⁰, and is specifically selected from the group consisting of hydrogen, C₁-C₄-alkyl and —C(═O)R¹⁰, where R¹⁰ has one of the general or in particular one of the preferred meanings given above and is specifically C₁-C₄-alkyl.

In case R²² is a substituent on an alkyl, alkenyl or alkynyl group, it is preferably selected from the group consisting of cyano, C₃-C₆-cycloalkyl, C₃-C₆-halocycloalkyl, —OR²³, —C(═O)N(R²⁴)R²⁵,

—C(═S)N(R²⁴)R²⁵, —C(═O)OR²³, —C(═O)R²⁰, phenyl which may be substituted by 1, 2, 3, 4 or 5 radicals selected from halogen, cyano, nitro, C₁-C₆-alkyl, C₁-C₆-haloalkyl, C₁-C₆-alkoxy and C₁-C₆-haloalkoxy, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals selected from halogen, cyano, nitro, C₁-C₆-alkyl, C₁-C₆-haloalkyl, C₁-C₆-alkoxy and C₁-C₆-haloalkoxy;
where
R²⁰ and R²³, independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹³; and
R²⁴ and R²⁵, independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹³.

In case R²² is a substituent on a cycloalkyl group, it is preferably selected from the group consisting of cyano, C₁-C₄-alkyl, C₁-C₄-haloalkyl, C₃-C₆-cycloalkyl, C₃-C₆-halocycloalkyl, —C(═O)N(R²⁴)R²⁵, —C(═S)N(R²⁴)R²⁵, —C(═O)OR²³, —C(═O)R²⁰, phenyl which may be substituted by 1, 2, 3, 4 or 5 radicals selected from halogen, cyano, nitro, C₁-C₆-alkyl, C₁-C₆-haloalkyl, C₁-C₆-alkoxy and C₁-C₆-haloalkoxy, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals selected from halogen, cyano, nitro, C₁-C₆-alkyl, C₁-C₆-haloalkyl, C₁-C₆-alkoxy and C₁-C₆-haloalkoxy; where
R²⁰ and R²³, independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹³; and
R²⁴ and R²⁵, independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R¹³.

R²³ is preferably selected from the group consisting of hydrogen, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₃-C₆-cycloalkyl-C₂-C₄-alkyl, phenyl which may be substituted by 1, 2, 3, 4 or 5 radicals selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals selected from halogen, cyano, nitro, C₁-C₆-alkyl, C₁-C₆-haloalkyl, C₁-C₆-alkoxy and C₁-C₆-haloalkoxy, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals selected from halogen, cyano, nitro, C₁-C₆-alkyl, C₁-C₆-haloalkyl, C₁-C₆-alkoxy and C₁-C₆-haloalkoxy.

R²⁴ and R²⁵, independently of each other and independently of each occurrence, are preferably selected from the group consisting of hydrogen, C₁-C₆-alkyl, C₁-C₆-haloalkyl, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals selected from halogen, cyano, nitro, C₁-C₆-alkyl, C₁-C₆-haloalkyl, C₁-C₆-alkoxy and C₁-C₆-haloalkoxy, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals selected from halogen, cyano, nitro, C₁-C₆-alkyl, C₁-C₆-haloalkyl, C₁-C₆-alkoxy and C₁-C₆-haloalkoxy;

or R²⁴ and R²⁵, together with the nitrogen atom to which they are bound, may form a 5- or 6-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy.

In a preferred embodiment, the compound of formula (I) is of the general formula (I-a)

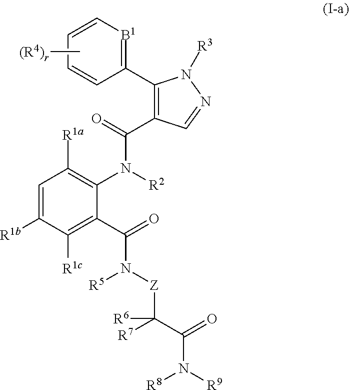

wherein
R^{1a}, R^{1b} and R^{1c} are selected from hydrogen and the group as defined for R¹; and
B¹, Z, R², R³, R⁴, R⁵, R⁶, R⁷, R⁸, R⁹ and r have one of the general meanings, or, in particular, one of the preferred meanings given above.

In particular, the compound of formula (I) is of the general formula (I-aa)

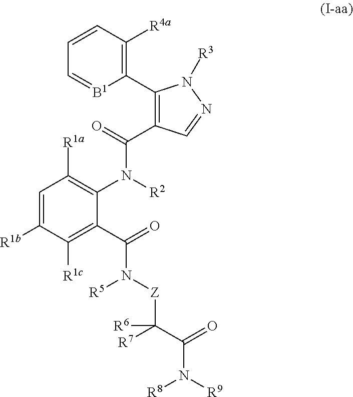

wherein
R^{1a}, R^{1b} and Ric are selected from hydrogen and the group as defined for R¹;
R^{4a} is selected from hydrogen and the group as defined for R⁴; and
B¹, Z, R², R³, R⁵, R⁶, R⁷, R⁸ and R⁹ have one of the general meanings, or, in particular, one of the preferred meanings given above.

Examples of preferred compounds are compounds of the following formulae Ia.1 to Ia.10, where the variables have one of the general or preferred meanings given above.

Examples of preferred compounds are the individual compounds compiled in the tables 1 to 960 below, Moreover, the meanings mentioned below for the individual variables in the tables are per se, independently of the combination in which they are mentioned, a particularly preferred embodiment of the substituents in question.

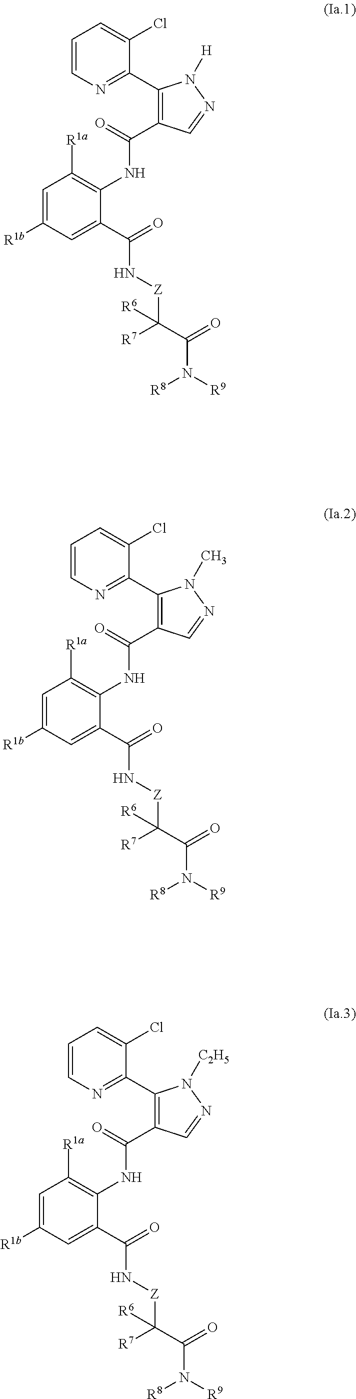

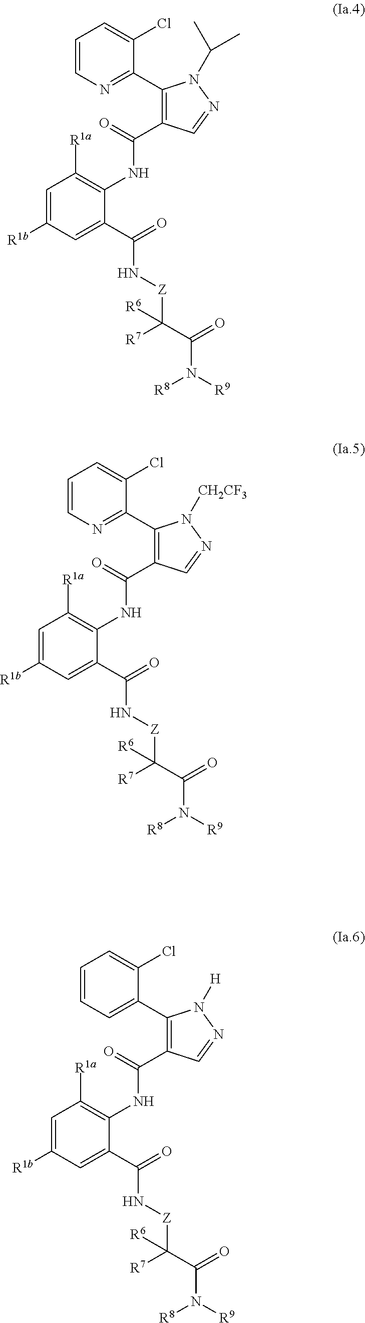

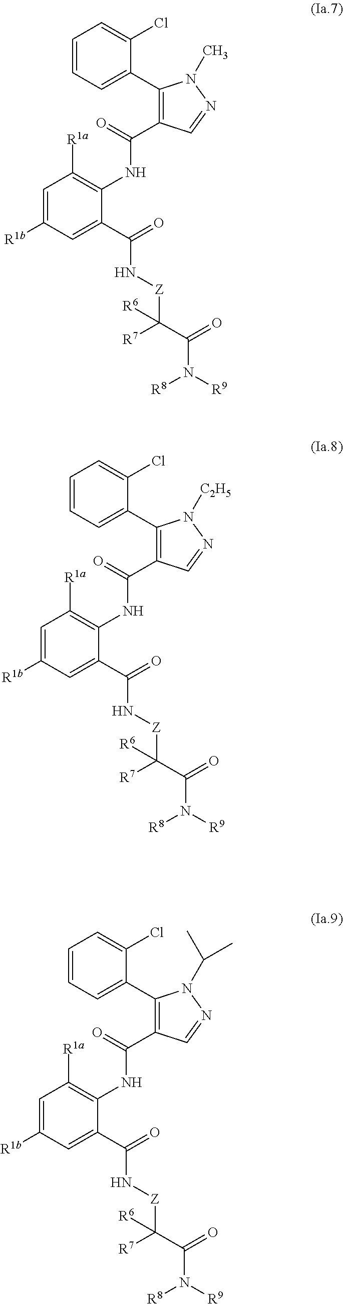

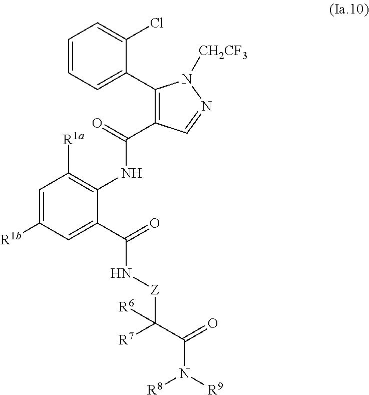

### Table 1

Compounds of the formula Ia.1 in which R^{1a} is methyl, R^{1b} is methyl, Z is O and the combination of R⁶, R⁷, R⁸ and R⁹ for a compound corresponds in each case to one row of Table A

### Table 2

Compounds of the formula Ia.1 in which R^{1a} is methyl, R^{1b} is Cl, Z is O and the combination of R⁶, R⁷, R⁸ and R⁹ for a compound corresponds in each case to one row of Table A

### Table 3

Compounds of the formula Ia.1 in which R^{1a} is Cl, R^{1b} is methyl, Z is O and the combination of R⁶, R⁷, R⁸ and R⁹ for a compound corresponds in each case to one row of Table A

### Table 4

Compounds of the formula Ia.1 in which R^{1a} is methyl, R^{1b} is Br, Z is O and the combination of R⁶, R⁷, R⁸ and R⁹ for a compound corresponds in each case to one row of Table A

### Table 5

Compounds of the formula Ia.1 in which R^{1a} is Br, R^{b} is methyl, Z is O and the combination of R⁶, R⁷, R⁸ and R⁹ for a compound corresponds in each case to one row of Table A

### Table 6

Compounds of the formula Ia.1 in which R^{1a} is methyl, R^{1b} is CN, Z is O and the combination of R⁶, R⁷, R⁸ and R⁹ for a compound corresponds in each case to one row of Table A

### Table 7

Compounds of the formula Ia.1 in which R^{1a} is CN, R^{1b} is methyl, Z is O and the combination of R⁶, R⁷, R⁸ and R⁹ for a compound corresponds in each case to one row of Table A

### Table 8

Compounds of the formula Ia.1 in which R^{1a} is Cl, R^{1b} is Cl, Z is O and the combination of R⁶, R⁷, R⁸ and R⁹ for a compound corresponds in each case to one row of Table A

### Table 9

Compounds of the formula Ia.1 in which R^{1a} is Cl, R^{1b} is Br, Z is O and the combination of R⁶, R⁷, R⁸ and R⁹ for a compound corresponds in each case to one row of Table A

### Table 10

Compounds of the formula Ia.1 in which R^{1a} is Br, R^{1b} is Cl, Z is O and the combination of R⁶, R⁷, R⁸ and R⁹ for a compound corresponds in each case to one row of Table A

### Table 11

Compounds of the formula Ia.1 in which R^{1a} is Cl, R^{1b} is CN, Z is O and the combination of R⁶, R⁷, R⁸ and R⁹ for a compound corresponds in each case to one row of Table A

### Table 12

Compounds of the formula Ia.1 in which R^{1a} is CN, R^{1b} is Cl, Z is O and the combination of R⁶, R⁷, R⁸ and R⁹ for a compound corresponds in each case to one row of Table A

### Table 13

Compounds of the formula Ia.1 in which R^{1a} is Br, R^{1b} is Br, Z is O and the combination of R⁶, R⁷, R⁸ and R⁹ for a compound corresponds in each case to one row of Table A

### Table 14

Compounds of the formula Ia.1 in which R^{1a} is Br, R^{1b} is CN, Z is O and the combination of R⁶, R⁷, R⁸ and R⁹ for a compound corresponds in each case to one row of Table A

### Table 15

Compounds of the formula Ia.1 in which R^{1a} is CN, R^{1b} is Br, Z is O and the combination of R⁶, R⁷, R⁸ and R⁹ for a compound corresponds in each case to one row of Table A

### Table 16

Compounds of the formula Ia.1 in which R^{1a} is CN, R^{1b} is CN, Z is O and the combination of R⁶, R⁷, R⁸ and R⁹ for a compound corresponds in each case to one row of Table A

### Table 17

Compounds of the formula Ia.1 in which R^{1a} is methyl, R^{1b} is methyl, Z is S and the combination of R⁶, R⁷, R⁸ and R⁹ for a compound corresponds in each case to one row of Table A

### Table 18

Compounds of the formula Ia.1 in which R^{1a} is methyl, R^{1b} is Cl, Z is S and the combination of R⁶, R⁷, R⁸ and R⁹ for a compound corresponds in each case to one row of Table A

### Table 19

Compounds of the formula Ia.1 in which R^{1a} is Cl, R^{1b} is methyl, Z is S and the combination of R⁶, R⁷, R⁸ and R⁹ for a compound corresponds in each case to one row of Table A

### Table 20

Compounds of the formula Ia.1 in which R^{1a} is methyl, R^{1b} is Br, Z is S and the combination of R⁶, R⁷, R⁸ and R⁹ for a compound corresponds in each case to one row of Table A

### Table 21

Compounds of the formula Ia.1 in which R^{1a} is Br, R^{1b} is methyl, Z is S and the combination of R⁶, R⁷, R⁸ and R⁹ for a compound corresponds in each case to one row of Table A

### Table 22

Compounds of the formula Ia.1 in which R^{1a} is methyl, R^{1b} is CN, Z is S and the combination of R⁶, R⁷, R⁸ and R⁹ for a compound corresponds in each case to one row of Table A

### Table 23

Compounds of the formula Ia.1 in which R^{1a} is CN, R^{1b} is methyl, Z is S and the combination of R⁶, R⁷, R⁸ and R⁹ for a compound corresponds in each case to one row of Table A

### Table 24

Compounds of the formula Ia.1 in which R^{1a} is Cl, R^{1b} is Cl, Z is S and the combination of R⁶, R⁷, R⁸ and R⁹ for a compound corresponds in each case to one row of Table A

### Table 25

Compounds of the formula Ia.1 in which R^{1a} is Cl, R^{1b} is Br, Z is S and the combination of R⁶, R⁷, R⁸ and R⁹ for a compound corresponds in each case to one row of Table A

### Table 26

Compounds of the formula Ia.1 in which R^{1a} is Br, R^{1b} is Cl, Z is S and the combination of R⁶, R⁷, R⁸ and R⁹ for a compound corresponds in each case to one row of Table A

### Table 27

Compounds of the formula Ia.1 in which R^{1a} is Cl, R^{1b} is CN, Z is S and the combination of R⁶, R⁷, R⁸ and R⁹ for a compound corresponds in each case to one row of Table A

### Table 28

Compounds of the formula Ia.1 in which R^{1a} is CN, R^{1b} is Cl, Z is S and the combination of R⁶, R⁷, R⁸ and R⁹ for a compound corresponds in each case to one row of Table A

### Table 29

Compounds of the formula Ia.1 in which R^{1a} is Br, R^{1b} is Br, Z is S and the combination of R⁶, R⁷, R⁸ and R⁹ for a compound corresponds in each case to one row of Table A

### Table 30

Compounds of the formula Ia.1 in which R^{1a} is Br, R^{1b} is CN, Z is S and the combination of R⁶, R⁷, R⁸ and R⁹ for a compound corresponds in each case to one row of Table A

### Table 31

Compounds of the formula Ia.1 in which R^{1a} is CN, R^{1b} is Br, Z is S and the combination of R⁶, R⁷, R⁸ and R⁹ for a compound corresponds in each case to one row of Table A

### Table 32

Compounds of the formula Ia.1 in which R^{1a} is CN, R^{1b} is CN, Z is S and the combination of R⁶, R⁷, R⁸ and R⁹ for a compound corresponds in each case to one row of Table A

### Table 33

Compounds of the formula Ia.1 in which R^{1a} is methyl, R^{1b} is methyl, Z is NH and the combination of R⁶, R⁷, R⁸ and R⁹ for a compound corresponds in each case to one row of Table A

### Table 34

Compounds of the formula Ia.1 in which R^{1a} is methyl, R^{1b} is Cl, Z is NH and the combination of R⁶, R⁷, R⁸ and R⁹ for a compound corresponds in each case to one row of Table A

### Table 35

Compounds of the formula Ia.1 in which R^{1a} is Cl, R^{1b} is methyl, Z is NH and the combination of R⁶, R⁷, R⁸ and R⁹ for a compound corresponds in each case to one row of Table A

### Table 36

Compounds of the formula Ia.1 in which R^{1a} is methyl, R^{1b} is Br, Z is NH and the combination of R⁶, R⁷, R⁸ and R⁹ for a compound corresponds in each case to one row of Table A

### Table 37

Compounds of the formula Ia.1 in which R^{1a} is Br, R^{1b} is methyl, Z is NH and the combination of R⁶, R⁷, R⁸ and R⁹ for a compound corresponds in each case to one row of Table A

### Table 38

Compounds of the formula Ia.1 in which R^{1a} is methyl, R^{1b} is CN, Z is NH and the combination of R⁶, R⁷, R⁸ and R⁹ for a compound corresponds in each case to one row of Table A

### Table 39

Compounds of the formula Ia.1 in which R^{1a} is CN, R^{1b} is methyl, Z is NH and the combination of R⁶, R⁷, R⁸ and R⁹ for a compound corresponds in each case to one row of Table A

### Table 40

Compounds of the formula Ia.1 in which R^{1a} is Cl, R^{1b} is Cl, Z is NH and the combination of R⁶, R⁷, R⁸ and R⁹ for a compound corresponds in each case to one row of Table A

### Table 41

Compounds of the formula Ia.1 in which R^{1a} is Cl, R^{1b} is Br, Z is NH and the combination of R⁶, R⁷, R⁸ and R⁹ for a compound corresponds in each case to one row of Table A

### Table 42

Compounds of the formula Ia.1 in which R^{1a} is Br, R^{1b} is Cl, Z is NH and the combination of R⁶, R⁷, R⁸ and R⁹ for a compound corresponds in each case to one row of Table A

### Table 43

Compounds of the formula Ia.1 in which R^{1a} is Cl, R^{1b} is CN, Z is NH and the combination of R⁶, R⁷, R⁸ and R⁹ for a compound corresponds in each case to one row of Table A

### Table 44

Compounds of the formula Ia.1 in which R^{1a} is CN, R^{1b} is Cl, Z is NH and the combination of R⁶, R⁷, R⁸ and R⁹ for a compound corresponds in each case to one row of Table A

### Table 45

Compounds of the formula Ia.1 in which R^{1a} is Br, R^{1b} is Br, Z is NH and the combination of R⁶, R⁷, R⁸ and R⁹ for a compound corresponds in each case to one row of Table A

### Table 46

Compounds of the formula Ia.1 in which R^{1a} is Br, R^{1b} is CN, Z is NH and the combination of R⁶, R⁷, R⁸ and R⁹ for a compound corresponds in each case to one row of Table A

### Table 47

Compounds of the formula Ia.1 in which R^{1a} is CN, R^{1b} is Br, Z is NH and the combination of R⁶, R⁷, R⁸ and R⁹ for a compound corresponds in each case to one row of Table A

### Table 48

Compounds of the formula Ia.1 in which R^{1a} is CN, R^{1b} is CN, Z is NH and the combination of R⁶, R⁷, R⁸ and R⁹ for a compound corresponds in each case to one row of Table A

### Table 49

Compounds of the formula Ia.1 in which R^{1a} is methyl, R^{1b} is methyl, Z is NCH₃ and the combination of R⁶, R⁷, R⁸ and R⁹ for a compound corresponds in each case to one row of Table A

### Table 50

Compounds of the formula Ia.1 in which R^{1a} is methyl, R^{1b} is Cl, Z is NCH₃ and the combination of R⁶, R⁷, R⁸ and R⁹ for a compound corresponds in each case to one row of Table A

### Table 51

Compounds of the formula Ia.1 in which R^{1a} is Cl, R^{1b} is methyl, Z is NCH₃ and the combination of R⁶, R⁷, R⁸ and R⁹ for a compound corresponds in each case to one row of Table A

### Table 52

Compounds of the formula Ia.1 in which R^{1a} is methyl, R^{1b} is Br, Z is NCH₃ and the combination of R⁶, R⁷, R⁸ and R⁹ for a compound corresponds in each case to one row of Table A

### Table 53

Compounds of the formula Ia.1 in which R^{1a} is Br, R^{1b} is methyl, Z is NCH₃ and the combination of R⁶, R⁷, R⁸ and R⁹ for a compound corresponds in each case to one row of Table A

### Table 54

Compounds of the formula Ia.1 in which R^{1a} is methyl, R^{1b} is CN, Z is NCH₃ and the combination of R⁶, R⁷, R⁸ and R⁹ for a compound corresponds in each case to one row of Table A

### Table 55

Compounds of the formula Ia.1 in which R^{1a} is CN, R^{1b} is methyl, Z is NCH₃ and the combination of R⁶, R⁷, R⁸ and R⁹ for a compound corresponds in each case to one row of Table A

### Table 56

Compounds of the formula Ia.1 in which R^{1a} is Cl, R^{1b} is Cl, Z is NCH₃ and the combination of R⁶, R⁷, R⁸ and R⁹ for a compound corresponds in each case to one row of Table A

### Table 57

Compounds of the formula Ia.1 in which R^{1a} is Cl, R^{1b} is Br, Z is NCH₃ and the combination of R⁶, R⁷, R⁸ and R⁹ for a compound corresponds in each case to one row of Table A

### Table 58

Compounds of the formula Ia.1 in which R^{1a} is Br, R^{1b} is Cl, Z is NCH₃ and the combination of R⁶, R⁷, R⁸ and R⁹ for a compound corresponds in each case to one row of Table A

### Table 59

Compounds of the formula Ia.1 in which R^{1a} is Cl, R^{1b} is CN, Z is NCH₃ and the combination of R⁶, R⁷, R⁸ and R⁹ for a compound corresponds in each case to one row of Table A

### Table 60

Compounds of the formula Ia.1 in which R^{1a} is CN, R^{1b} is Cl, Z is NCH₃ and the combination of R⁶, R⁷, R⁸ and R⁹ for a compound corresponds in each case to one row of Table A

### Table 61

Compounds of the formula Ia.1 in which R^{1a} is Br, R^{1b} is Br, Z is NCH₃ and the combination of R⁶, R⁷, R⁸ and R⁹ for a compound corresponds in each case to one row of Table A

### Table 62

Compounds of the formula Ia.1 in which R^{1a} is Br, R^{1b} is CN, Z is NCH₃ and the combination of R⁶, R⁷, R⁸ and R⁹ for a compound corresponds in each case to one row of Table A

### Table 63

Compounds of the formula Ia.1 in which R^{1a} is CN, R^{1b} is Br, Z is NCH₃ and the combination of R⁶, R⁷, R⁸ and R⁹ for a compound corresponds in each case to one row of Table A

### Table 64

Compounds of the formula Ia.1 in which R^{1a} is CN, R^{1b} is CN, Z is NCH₃ and the combination of R⁶, R⁷, R⁸ and R⁹ for a compound corresponds in each case to one row of Table A

### Table 65

Compounds of the formula Ia.1 in which R^{1a} is methyl, R^{1b} is methyl, Z is NCH₂CH₃ and the combination of R⁶, R⁷, R⁸ and R⁹ for a compound corresponds in each case to one row of Table A

### Table 66

Compounds of the formula Ia.1 in which R^{1a} is methyl, R^{1b} is Cl, Z is NCH₂CH₃ and the combination of R⁶, R⁷, R⁸ and R⁹ for a compound corresponds in each case to one row of Table A

### Table 67

Compounds of the formula Ia.1 in which R^{1a} is Cl, R^{1b} is methyl, Z is NCH₂CH₃ and the combination of R⁶, R⁷, R⁸ and R⁹ for a compound corresponds in each case to one row of Table A

### Table 68

Compounds of the formula Ia.1 in which R^{1a} is methyl, R^{1b} is Br, Z is NCH₂CH₃ and the combination of R⁶, R⁷, R⁸ and R⁹ for a compound corresponds in each case to one row of Table A

### Table 69

Compounds of the formula Ia.1 in which R^{1a} is Br, R^{1b} is methyl, Z is NCH₂CH₃ and the combination of R⁶, R⁷, R⁸ and R⁹ for a compound corresponds in each case to one row of Table A

### Table 70

Compounds of the formula Ia.1 in which R^{1a} is methyl, R^{1b} is CN, Z is NCH₂CH₃ and the combination of R⁶, R⁷, R⁸ and R⁹ for a compound corresponds in each case to one row of Table A

### Table 71

Compounds of the formula Ia.1 in which R^{1a} is CN, R^{1b} is methyl, Z is NCH₂CH₃ and the combination of R⁶, R⁷, R⁸ and R⁹ for a compound corresponds in each case to one row of Table A

### Table 72

Compounds of the formula Ia.1 in which R^{1a} is Cl, R^{1b} is Cl, Z is NCH₂CH₃ and the combination of R⁶, R⁷, R⁸ and R⁹ for a compound corresponds in each case to one row of Table A

### Table 73

Compounds of the formula Ia.1 in which R^{1a} is Cl, R^{1b} is Br, Z is NCH₂CH₃ and the combination of R⁶, R⁷, R⁸ and R⁹ for a compound corresponds in each case to one row of Table A

### Table 74

Compounds of the formula Ia.1 in which R^{1a} is Br, R^{1b} is Cl, Z is NCH₂CH₃ and the combination of R⁶, R⁷, R⁸ and R⁹ for a compound corresponds in each case to one row of Table A

### Table 75

Compounds of the formula Ia.1 in which R^{1a} is Cl, R^{1b} is CN, Z is NCH₂CH₃ and the combination of R⁶, R⁷, R⁸ and R⁹ for a compound corresponds in each case to one row of Table A

### Table 76

Compounds of the formula Ia.1 in which R^{1a} is CN, R^{1b} is Cl, Z is NCH₂CH₃ and the combination of R⁶, R⁷, R⁸ and R⁹ for a compound corresponds in each case to one row of Table A

### Table 77

Compounds of the formula Ia.1 in which R^{1a} is Br, R^{1b} is Br, Z is NCH₂CH₃ and the combination of R⁶, R⁷, R⁸ and R⁹ for a compound corresponds in each case to one row of Table A

### Table 78

Compounds of the formula Ia.1 in which R^{1a} is Br, R^{1b} is CN, Z is NCH₂CH₃ and the combination of R⁶, R⁷, R⁸ and R⁹ for a compound corresponds in each case to one row of Table A

### Table 79

Compounds of the formula Ia.1 in which R^{1a} is CN, R^{1b} is Br, Z is NCH₂CH₃ and the combination of R⁶, R⁷, R⁸ and R⁹ for a compound corresponds in each case to one row of Table A

### Table 80

Compounds of the formula Ia.1 in which R^{1a} is CN, R^{1b} is CN, Z is NCH₂CH₃ and the combination of R⁶, R⁷, R⁸ and R⁹ for a compound corresponds in each case to one row of Table A

### Table 81

Compounds of the formula Ia.1 in which R^{1a} is methyl, R^{1b} is methyl, Z is NCH(CH₃)₂ and the combination of R⁶, R⁷, R⁸ and R⁹ for a compound corresponds in each case to one row of Table A

### Table 82

Compounds of the formula Ia.1 in which R^{1a} is methyl, R^{1b} is Cl, Z is NCH(CH₃)₂ and the combination of R⁶, R⁷, R⁸ and R⁹ for a compound corresponds in each case to one row of Table A

### Table 83

Compounds of the formula Ia.1 in which R^{1a} is Cl, R^{1b} is methyl, Z is NCH(CH₃)₂ and the combination of R⁶, R⁷, R⁸ and R⁹ for a compound corresponds in each case to one row of Table A

### Table 84

Compounds of the formula Ia.1 in which R^{1a} is methyl, R^{1b} is Br, Z is NCH(CH₃)₂ and the combination of R⁶, R⁷, R⁸ and R⁹ for a compound corresponds in each case to one row of Table A

### Table 85

Compounds of the formula Ia.1 in which R^{1a} is Br, R^{1b} is methyl, Z is NCH(CH₃)₂ and the combination of R⁶, R⁷, R⁸ and R⁹ for a compound corresponds in each case to one row of Table A

### Table 86

Compounds of the formula Ia.1 in which R^{1a} is methyl, R^{1b} is CN, Z is NCH(CH₃)₂ and the combination of R⁶, R⁷, R⁸ and R⁹ for a compound corresponds in each case to one row of Table A

### Table 87

Compounds of the formula Ia.1 in which R^{1a} is CN, R^{1b} is methyl, Z is NCH(CH₃)₂ and the combination of R⁶, R⁷, R⁸ and R⁹ for a compound corresponds in each case to one row of Table A

### Table 88

Compounds of the formula Ia.1 in which R^{1a} is Cl, R^{1b} is Cl, Z is NCH(CH₃)₂ and the combination of R⁶, R⁷, R⁸ and R⁹ for a compound corresponds in each case to one row of Table A

### Table 89

Compounds of the formula Ia.1 in which R^{1a} is Cl, R^{1b} is Br, Z is NCH(CH₃)₂ and the combination of R⁶, R⁷, R⁸ and R⁹ for a compound corresponds in each case to one row of Table A

### Table 90

Compounds of the formula Ia.1 in which R^{1a} is Br, R^{1b} is Cl, Z is NCH(CH₃)₂ and the combination of R⁶, R⁷, R⁸ and R⁹ for a compound corresponds in each case to one row of Table A

### Table 91

Compounds of the formula Ia.1 in which R^{1a} is Cl, R^{1b} is CN, Z is NCH(CH₃)₂ and the combination of R⁶, R⁷, R⁸ and R⁹ for a compound corresponds in each case to one row of Table A

### Table 92

Compounds of the formula Ia.1 in which R^{1a} is CN, R^{1b} is Cl, Z is NCH(CH₃)₂ and the combination of R⁶, R⁷, R⁸ and R⁹ for a compound corresponds in each case to one row of Table A

### Table 93

Compounds of the formula Ia.1 in which R^{1a} is Br, R^{1b} is Br, Z is NCH(CH₃)₂ and the combination of R⁶, R⁷, R⁸ and R⁹ for a compound corresponds in each case to one row of Table A

### Table 94

Compounds of the formula Ia.1 in which R^{1a} is Br, R^{1b} is CN, Z is NCH(CH₃)₂ and the combination of R⁶, R⁷, R⁸ and R⁹ for a compound corresponds in each case to one row of Table A

### Table 95

Compounds of the formula Ia.1 in which R^{1a} is CN, R^{1b} is Br, Z is NCH(CH₃)₂ and the combination of R⁶, R⁷, R⁸ and R⁹ for a compound corresponds in each case to one row of Table A

### Table 96

Compounds of the formula Ia.1 in which R^{1a} is CN, R^{1b} is CN, Z is NCH(CH₃)₂ and the combination of R⁶, R⁷, R⁸ and R⁹ for a compound corresponds in each case to one row of Table A

### Tables 97 to 192

Compounds of the formula Ia.2 in which the combination of Ra, R^{1b} and Z is as defined in any of tables 1 to 96 and the combination of R⁶, R⁷, R⁸ and R⁹ for a compound corresponds in each case to one row of Table A

### Tables 193 to 288

Compounds of the formula Ia.3 in which the combination of R¹, R^{1b} and Z is as defined in any of tables 1 to 96 and the combination of R⁶, R⁷, R⁸ and R⁹ for a compound corresponds in each case to one row of Table A

### Tables 289 to 384

Compounds of the formula Ia.4 in which the combination of R^{1a}, R^{1b} and Z is as defined in any of tables 1 to 96 and the combination of R⁶, R⁷, R⁸ and R⁹ for a compound corresponds in each case to one row of Table A

### Tables 385 to 480

Compounds of the formula Ia.5 in which the combination of R¹, R^{1b} and Z is as defined in any of tables 1 to 96 and the combination of R⁶, R⁷, R⁸ and R⁹ for a compound corresponds in each case to one row of Table A

### Tables 481 to 576

Compounds of the formula Ia.6 in which the combination of R^{1a}, R^{1b} and Z is as defined in any of tables 1 to 96 and the combination of R⁶, R⁷, R⁸ and R⁹ for a compound corresponds in each case to one row of Table A

### Tables 577 to 672

Compounds of the formula Ia.7 in which the combination of R^{1a}, R^{1b} and Z is as defined in any of tables 1 to 96 and the combination of R⁶, R⁷, R⁸ and R⁹ for a compound corresponds in each case to one row of Table A

### Tables 673 to 768

Compounds of the formula Ia.8 in which the combination of R^{1a}, R^{1b} and Z is as defined in any of tables 1 to 96 and the combination of R⁶, R⁷, R⁸ and R⁹ for a compound corresponds in each case to one row of Table A

### Tables 769 to 864

Compounds of the formula Ia.9 in which the combination of R¹, R^{1b} and Z is as defined in any of tables 1 to 96 and the combination of R⁶, R⁷, R⁸ and R⁹ for a compound corresponds in each case to one row of Table A

### Tables 865 to 960

Compounds of the formula Ia.10 in which the combination of R^{1a}, R^{1b} and Z is as defined in any of tables 1 to 96 and the combination of R⁶, R⁷, R⁸ and R⁹ for a compound corresponds in each case to one row of Table A

TABLE A

| No. | R⁶ | R⁷ | R⁸ | R⁹ |
|---|---|---|---|---|
| A-1 | H | H | H | H |
| A-2 | H | H | H | CH₃ |
| A-3 | H | H | H | C₂H₅ |
| A-4 | H | H | H | CH(CH₃)₂ |
| A-5 | H | H | H | ^{c}(CH₂)₃ |
| A-6 | H | H | H | CH₂CF₃ |
| A-7 | H | H | H | CH₂CH═CH₂ |
| A-8 | H | H | H | C₆H₅ |
| A-9 | H | H | H | CH₂C₆H₅ |
| A-10 | H | H | CH₃ | H |
| A-11 | H | H | CH₃ | CH₃ |
| A-12 | H | H | CH₃ | C₂H₅ |
| A-13 | H | H | CH₃ | CH(CH₃)₂ |
| A-14 | H | H | CH₃ | ^{c}(CH₂)₃ |
| A-15 | H | H | CH₃ | CH₂CF₃ |
| A-16 | H | H | CH₃ | CH₂CH═CH₂ |
| A-17 | H | H | CH₃ | C₆H₅ |
| A-18 | H | H | CH₃ | CH₂C₆H₅ |
| A-19 | H | H | C₂H₅ | H |
| A-20 | H | H | C₂H₅ | CH₃ |
| A-21 | H | H | C₂H₅ | C₂H₅ |
| A-22 | H | H | C₂H₅ | CH(CH₃)₂ |
| A-23 | H | H | C₂H₅ | ^{c}(CH₂)₃ |
| A-24 | H | H | C₂H₅ | CH₂CF₃ |
| A-25 | H | H | C₂H₅ | CH₂CH═CH₂ |
| A-26 | H | H | C₂H₅ | C₆H₅ |
| A-27 | H | H | C₂H₅ | CH₂C₆H₅ |
| A-28 | CH₃ | H | H | H |
| A-29 | CH₃ | H | H | CH₃ |
| A-30 | CH₃ | H | H | C₂H₅ |
| A-31 | CH₃ | H | H | CH(CH₃)₂ |
| A-32 | CH₃ | H | H | ^{c}(CH₂)₃ |
| A-33 | CH₃ | H | H | CH₂CF₃ |
| A-34 | CH₃ | H | H | CH₂CH═CH₂ |
| A-35 | CH₃ | H | H | C₆H₅ |
| A-36 | CH₃ | H | H | CH₂C₆H₅ |
| A-37 | CH₃ | H | CH₃ | H |
| A-38 | CH₃ | H | CH₃ | CH₃ |
| A-39 | CH₃ | H | CH₃ | C₂H₅ |
| A-40 | CH₃ | H | CH₃ | CH(CH₃)₂ |
| A-41 | CH₃ | H | CH₃ | ^{c}(CH₂)₃ |
| A-42 | CH₃ | H | CH₃ | CH₂CF₃ |
| A-43 | CH₃ | H | CH₃ | CH₂CH═CH₂ |
| A-44 | CH₃ | H | CH₃ | C₆H₅ |
| A-45 | CH₃ | H | CH₃ | CH₂C₆H₅ |
| A-46 | CH₃ | H | C₂H₅ | H |
| A-47 | CH₃ | H | C₂H₅ | CH₃ |
| A-48 | CH₃ | H | C₂H₅ | C₂H₅ |
| A-49 | CH₃ | H | C₂H₅ | CH(CH₃)₂ |
| A-50 | CH₃ | H | C₂H₅ | ^{c}(CH₂)₃ |
| A-51 | CH₃ | H | C₂H₅ | CH₂CF₃ |
| A-52 | CH₃ | H | C₂H₅ | CH₂CH═CH₂ |
| A-53 | CH₃ | H | C₂H₅ | C₆H₅ |
| A-54 | CH₃ | H | C₂H₅ | CH₂C₆H₅ |
| A-55 | C₂H₅ | H | H | H |
| A-56 | C₂H₅ | H | H | CH₃ |
| A-57 | C₂H₅ | H | H | C₂H₅ |
| A-58 | C₂H₅ | H | H | CH(CH₃)₂ |
| A-59 | C₂H₅ | H | H | ^{c}(CH₂)₃ |
| A-60 | C₂H₅ | H | H | CH₂CF₃ |
| A-61 | C₂H₅ | H | H | CH₂CH═CH₂ |
| A-62 | C₂H₅ | H | H | C₆H₅ |
| A-63 | C₂H₅ | H | H | CH₂C₆H₅ |
| A-64 | C₂H₅ | H | CH₃ | H |
| A-65 | C₂H₅ | H | CH₃ | CH₃ |
| A-66 | C₂H₅ | H | CH₃ | C₂H₅ |
| A-67 | C₂H₅ | H | CH₃ | CH(CH₃)₂ |
| A-68 | C₂H₅ | H | CH₃ | ^{c}(CH₂)₃ |
| A-69 | C₂H₅ | H | CH₃ | CH₂CF₃ |
| A-70 | C₂H₅ | H | CH₃ | CH₂CH═CH₂ |
| A-71 | C₂H₅ | H | CH₃ | C₆H₅ |
| A-72 | C₂H₅ | H | CH₃ | CH₂C₆H₅ |
| A-73 | C₂H₅ | H | C₂H₅ | H |
| A-74 | C₂H₅ | H | C₂H₅ | CH₃ |
| A-75 | C₂H₅ | H | C₂H₅ | C₂H₅ |
| A-76 | C₂H₅ | H | C₂H₅ | CH(CH₃)₂ |
| A-77 | C₂H₅ | H | C₂H₅ | ^{c}(CH₂)₃ |
| A-78 | C₂H₅ | H | C₂H₅ | CH₂CF₃ |
| A-79 | C₂H₅ | H | C₂H₅ | CH₂CH═CH₂ |
| A-80 | C₂H₅ | H | C₂H₅ | C₆H₅ |
| A-81 | C₂H₅ | H | C₂H₅ | CH₂C₆H₅ |
| A-82 | CH(CH₃)₂ | H | H | H |
| A-83 | CH(CH₃)₂ | H | H | CH₃ |
| A-84 | CH(CH₃)₂ | H | H | C₂H₅ |
| A-85 | CH(CH₃)₂ | H | H | CH(CH₃)₂ |
| A-86 | CH(CH₃)₂ | H | H | ^{c}(CH₂)₃ |
| A-87 | CH(CH₃)₂ | H | H | CH₂CF₃ |
| A-88 | CH(CH₃)₂ | H | H | CH₂CH═CH₂ |
| A-89 | CH(CH₃)₂ | H | H | C₆H₅ |
| A-90 | CH(CH₃)₂ | H | H | CH₂C₆H₅ |
| A-91 | CH(CH₃)₂ | H | CH₃ | H |
| A-92 | CH(CH₃)₂ | H | CH₃ | CH₃ |
| A-93 | CH(CH₃)₂ | H | CH₃ | C₂H₅ |
| A-94 | CH(CH₃)₂ | H | CH₃ | CH(CH₃)₂ |
| A-95 | CH(CH₃)₂ | H | CH₃ | ^{c}(CH₂)₃ |
| A-96 | CH(CH₃)₂ | H | CH₃ | CH₂CF₃ |
| A-97 | CH(CH₃)₂ | H | CH₃ | CH₂CH═CH₂ |
| A-98 | CH(CH₃)₂ | H | CH₃ | C₆H₅ |
| A-99 | CH(CH₃)₂ | H | CH₃ | CH₂C₆H₅ |
| A-100 | CH(CH₃)₂ | H | C₂H₅ | H |
| A-101 | CH(CH₃)₂ | H | C₂H₅ | CH₃ |
| A-102 | CH(CH₃)₂ | H | C₂H₅ | C₂H₅ |
| A-103 | CH(CH₃)₂ | H | C₂H₅ | CH(CH₃)₂ |
| A-104 | CH(CH₃)₂ | H | C₂H₅ | ^{c}(CH₂)₃ |
| A-105 | CH(CH₃)₂ | H | C₂H₅ | CH₂CF₃ |
| A-106 | CH(CH₃)₂ | H | C₂H₅ | CH₂CH═CH₂ |
| A-107 | CH(CH₃)₂ | H | C₂H₅ | C₆H₅ |
| A-108 | CH(CH₃)₂ | H | C₂H₅ | CH₂C₆H₅ |
| A-109 | CH₃ | CH₃ | H | H |
| A-110 | CH₃ | CH₃ | H | CH₃ |
| A-111 | CH₃ | CH₃ | H | C₂H₅ |
| A-112 | CH₃ | CH₃ | H | CH(CH₃)₂ |
| A-113 | CH₃ | CH₃ | H | ^{c}(CH₂)₃ |
| A-114 | CH₃ | CH₃ | H | CH₂CF₃ |
| A-115 | CH₃ | CH₃ | H | CH₂CH═CH₂ |
| A-116 | CH₃ | CH₃ | H | C₆H₅ |
| A-117 | CH₃ | CH₃ | H | CH₂C₆H₅ |
| A-118 | CH₃ | CH₃ | CH₃ | H |
| A-119 | CH₃ | CH₃ | CH₃ | CH₃ |
| A-120 | CH₃ | CH₃ | CH₃ | C₂H₅ |
| A-121 | CH₃ | CH₃ | CH₃ | CH(CH₃)₂ |
| A-122 | CH₃ | CH₃ | CH₃ | ^{c}(CH₂)₃ |
| A-123 | CH₃ | CH₃ | CH₃ | CH₂CF₃ |
| A-124 | CH₃ | CH₃ | CH₃ | CH₂CH═CH₂ |
| A-125 | CH₃ | CH₃ | CH₃ | C₆H₅ |
| A-126 | CH₃ | CH₃ | CH₃ | CH₂C₆H₅ |
| A-127 | CH₃ | CH₃ | C₂H₅ | H |
| A-128 | CH₃ | CH₃ | C₂H₅ | CH₃ |
| A-129 | CH₃ | CH₃ | C₂H₅ | C₂H₅ |
| A-130 | CH₃ | CH₃ | C₂H₅ | CH(CH₃)₂ |
| A-131 | CH₃ | CH₃ | C₂H₅ | ^{c}(CH₂)₃ |
| A-132 | CH₃ | CH₃ | C₂H₅ | CH₂CF₃ |
| A-133 | CH₃ | CH₃ | C₂H₅ | CH₂CH═CH₂ |
| A-134 | CH₃ | CH₃ | C₂H₅ | C₆H₅ |
| A-135 | CH₃ | CH₃ | C₂H₅ | CH₂C₆H₅ |
| A-136 | C₂H₅ | CH₃ | H | H |
| A-137 | C₂H₅ | CH₃ | H | CH₃ |
| A-138 | C₂H₅ | CH₃ | H | C₂H₅ |
| A-139 | C₂H₅ | CH₃ | H | CH(CH₃)₂ |
| A-140 | C₂H₅ | CH₃ | H | ^{c}(CH₂)₃ |
| A-141 | C₂H₅ | CH₃ | H | CH₂CF₃ |
| A-142 | C₂H₅ | CH₃ | H | CH₂CH═CH₂ |
| A-143 | C₂H₅ | CH₃ | H | C₆H₅ |
| A-144 | C₂H₅ | CH₃ | H | CH₂C₆H₅ |
| A-145 | C₂H₅ | CH₃ | CH₃ | H |
| A-146 | C₂H₅ | CH₃ | CH₃ | CH₃ |
| A-147 | C₂H₅ | CH₃ | CH₃ | C₂H₅ |
| A-148 | C₂H₅ | CH₃ | CH₃ | CH(CH₃)₂ |
| A-149 | C₂H₅ | CH₃ | CH₃ | ^{c}(CH₂)₃ |
| A-150 | C₂H₅ | CH₃ | CH₃ | CH₂CF₃ |
| A-151 | C₂H₅ | CH₃ | CH₃ | CH₂CH═CH₂ |
| A-152 | C₂H₅ | CH₃ | CH₃ | C₆H₅ |
| A-153 | C₂H₅ | CH₃ | CH₃ | CH₂C₆H₅ |
| A-154 | C₂H₅ | CH₃ | C₂H₅ | H |
| A-155 | C₂H₅ | CH₃ | C₂H₅ | CH₃ |
| A-156 | C₂H₅ | CH₃ | C₂H₅ | C₂H₅ |
| A-157 | C₂H₅ | CH₃ | C₂H₅ | CH(CH₃)₂ |
| A-158 | C₂H₅ | CH₃ | C₂H₅ | ^{c}(CH₂)₃ |
| A-159 | C₂H₅ | CH₃ | C₂H₅ | CH₂CF₃ |
| A-160 | C₂H₅ | CH₃ | C₂H₅ | CH₂CH═CH₂ |
| A-161 | C₂H₅ | CH₃ | C₂H₅ | C₆H₅ |
| A-162 | C₂H₅ | CH₃ | C₂H₅ | CH₂C₆H₅ |
| A-163 | CH(CH₃)₂ | CH₃ | H | H |
| A-164 | CH(CH₃)₂ | CH₃ | H | CH₃ |
| A-165 | CH(CH₃)₂ | CH₃ | H | C₂H₅ |
| A-166 | CH(CH₃)₂ | CH₃ | H | CH(CH₃)₂ |
| A-167 | CH(CH₃)₂ | CH₃ | H | ^{c}(CH₂)₃ |
| A-168 | CH(CH₃)₂ | CH₃ | H | CH₂CF₃ |
| A-169 | CH(CH₃)₂ | CH₃ | H | CH₂CH═CH₂ |
| A-170 | CH(CH₃)₂ | CH₃ | H | C₆H₅ |
| A-171 | CH(CH₃)₂ | CH₃ | H | CH₂C₆H₅ |
| A-172 | CH(CH₃)₂ | CH₃ | CH₃ | H |
| A-173 | CH(CH₃)₂ | CH₃ | CH₃ | CH₃ |
| A-174 | CH(CH₃)₂ | CH₃ | CH₃ | C₂H₅ |
| A-175 | CH(CH₃)₂ | CH₃ | CH₃ | CH(CH₃)₂ |
| A-176 | CH(CH₃)₂ | CH₃ | CH₃ | ^{c}(CH₂)₃ |
| A-177 | CH(CH₃)₂ | CH₃ | CH₃ | CH₂CF₃ |
| A-178 | CH(CH₃)₂ | CH₃ | CH₃ | CH₂CH═CH₂ |
| A-179 | CH(CH₃)₂ | CH₃ | CH₃ | C₆H₅ |
| A-180 | CH(CH₃)₂ | CH₃ | CH₃ | CH₂C₆H₅ |
| A-181 | CH(CH₃)₂ | CH₃ | C₂H₅ | H |
| A-182 | CH(CH₃)₂ | CH₃ | C₂H₅ | CH₃ |
| A-183 | CH(CH₃)₂ | CH₃ | C₂H₅ | C₂H₅ |
| A-184 | CH(CH₃)₂ | CH₃ | C₂H₅ | CH(CH₃)₂ |
| A-185 | CH(CH₃)₂ | CH₃ | C₂H₅ | ^{c}(CH₂)₃ |
| A-186 | CH(CH₃)₂ | CH₃ | C₂H₅ | CH₂CF₃ |
| A-187 | CH(CH₃)₂ | CH₃ | C₂H₅ | CH₂CH═CH₂ |
| A-188 | CH(CH₃)₂ | CH₃ | C₂H₅ | C₆H₅ |
| A-189 | CH(CH₃)₂ | CH₃ | C₂H₅ | CH₂C₆H₅ |
| A-190 | C₂H₅ | C₂H₅ | H | H |
| A-191 | C₂H₅ | C₂H₅ | H | CH₃ |
| A-192 | C₂H₅ | C₂H₅ | H | C₂H₅ |
| A-193 | C₂H₅ | C₂H₅ | H | CH(CH₃)₂ |
| A-194 | C₂H₅ | C₂H₅ | H | ^{c}(CH₂)₃ |
| A-195 | C₂H₅ | C₂H₅ | H | CH₂CF₃ |
| A-196 | C₂H₅ | C₂H₅ | H | CH₂CH═CH₂ |
| A-197 | C₂H₅ | C₂H₅ | H | C₆H₅ |
| A-198 | C₂H₅ | C₂H₅ | H | CH₂C₆H₅ |
| A-199 | C₂H₅ | C₂H₅ | CH₃ | H |
| A-200 | C₂H₅ | C₂H₅ | CH₃ | CH₃ |
| A-201 | C₂H₅ | C₂H₅ | CH₃ | C₂H₅ |
| A-202 | C₂H₅ | C₂H₅ | CH₃ | CH(CH₃)₂ |
| A-203 | C₂H₅ | C₂H₅ | CH₃ | ^{c}(CH₂)₃ |
| A-204 | C₂H₅ | C₂H₅ | CH₃ | CH₂CF₃ |
| A-205 | C₂H₅ | C₂H₅ | CH₃ | CH₂CH═CH₂ |
| A-206 | C₂H₅ | C₂H₅ | CH₃ | C₆H₅ |
| A-207 | C₂H₅ | C₂H₅ | CH₃ | CH₂C₆H₅ |
| A-208 | C₂H₅ | C₂H₅ | C₂H₅ | H |
| A-209 | C₂H₅ | C₂H₅ | C₂H₅ | CH₃ |
| A-210 | C₂H₅ | C₂H₅ | C₂H₅ | C₂H₅ |
| A-211 | C₂H₅ | C₂H₅ | C₂H₅ | CH(CH₃)₂ |
| A-212 | C₂H₅ | C₂H₅ | C₂H₅ | ^{c}(CH₂)₃ |
| A-213 | C₂H₅ | C₂H₅ | C₂H₅ | CH₂CF₃ |
| A-214 | C₂H₅ | C₂H₅ | C₂H₅ | CH₂CH═CH₂ |
| A-215 | C₂H₅ | C₂H₅ | C₂H₅ | C₆H₅ |
| A-216 | C₂H₅ | C₂H₅ | C₂H₅ | CH₂C₆H₅ |
| A-217 | CH(CH₃)₂ | C₂H₅ | H | H |
| A-218 | CH(CH₃)₂ | C₂H₅ | H | CH₃ |
| A-219 | CH(CH₃)₂ | C₂H₅ | H | C₂H₅ |
| A-220 | CH(CH₃)₂ | C₂H₅ | H | CH(CH₃)₂ |
| A-221 | CH(CH₃)₂ | C₂H₅ | H | ^{c}(CH₂)₃ |
| A-222 | CH(CH₃)₂ | C₂H₅ | H | CH₂CF₃ |
| A-223 | CH(CH₃)₂ | C₂H₅ | H | CH₂CH═CH₂ |
| A-224 | CH(CH₃)₂ | C₂H₅ | H | C₆H₅ |
| A-225 | CH(CH₃)₂ | C₂H₅ | H | CH₂C₆H₅ |
| A-226 | CH(CH₃)₂ | C₂H₅ | CH₃ | H |
| A-227 | CH(CH₃)₂ | C₂H₅ | CH₃ | CH₃ |
| A-228 | CH(CH₃)₂ | C₂H₅ | CH₃ | C₂H₅ |
| A-229 | CH(CH₃)₂ | C₂H₅ | CH₃ | CH(CH₃)₂ |
| A-230 | CH(CH₃)₂ | C₂H₅ | CH₃ | ^{c}(CH₂)₃ |
| A-231 | CH(CH₃)₂ | C₂H₅ | CH₃ | CH₂CF₃ |
| A-232 | CH(CH₃)₂ | C₂H₅ | CH₃ | CH₂CH═CH₂ |
| A-233 | CH(CH₃)₂ | C₂H₅ | CH₃ | C₆H₅ |
| A-234 | CH(CH₃)₂ | C₂H₅ | CH₃ | CH₂C₆H₅ |
| A-235 | CH(CH₃)₂ | C₂H₅ | C₂H₅ | H |
| A-236 | CH(CH₃)₂ | C₂H₅ | C₂H₅ | CH₃ |
| A-237 | CH(CH₃)₂ | C₂H₅ | C₂H₅ | C₂H₅ |
| A-238 | CH(CH₃)₂ | C₂H₅ | C₂H₅ | CH(CH₃)₂ |
| A-239 | CH(CH₃)₂ | C₂H₅ | C₂H₅ | ^{c}(CH₂)₃ |
| A-240 | CH(CH₃)₂ | C₂H₅ | C₂H₅ | CH₂CF₃ |
| A-241 | CH(CH₃)₂ | C₂H₅ | C₂H₅ | CH₂CH═CH₂ |
| A-242 | CH(CH₃)₂ | C₂H₅ | C₂H₅ | C₆H₅ |
| A-243 | CH(CH₃)₂ | C₂H₅ | C₂H₅ | CH₂C₆H₅ |
| A-244 | CH(CH₃)₂ | CH(CH₃)₂ | H | H |
| A-245 | CH(CH₃)₂ | CH(CH₃)₂ | H | CH₃ |
| A-246 | CH(CH₃)₂ | CH(CH₃)₂ | H | C₂H₅ |
| A-247 | CH(CH₃)₂ | CH(CH₃)₂ | H | CH(CH₃)₂ |
| A-248 | CH(CH₃)₂ | CH(CH₃)₂ | H | ^{c}(CH₂)₃ |
| A-249 | CH(CH₃)₂ | CH(CH₃)₂ | H | CH₂CF₃ |
| A-250 | CH(CH₃)₂ | CH(CH₃)₂ | H | CH₂CH═CH₂ |
| A-251 | CH(CH₃)₂ | CH(CH₃)₂ | H | C₆H₅ |
| A-252 | CH(CH₃)₂ | CH(CH₃)₂ | H | CH₂C₆H₅ |
| A-253 | CH(CH₃)₂ | CH(CH₃)₂ | CH₃ | H |
| A-254 | CH(CH₃)₂ | CH(CH₃)₂ | CH₃ | CH₃ |
| A-255 | CH(CH₃)₂ | CH(CH₃)₂ | CH₃ | C₂H₅ |
| A-256 | CH(CH₃)₂ | CH(CH₃)₂ | CH₃ | CH(CH₃)₂ |
| A-257 | CH(CH₃)₂ | CH(CH₃)₂ | CH₃ | ^{c}(CH₂)₃ |
| A-258 | CH(CH₃)₂ | CH(CH₃)₂ | CH₃ | CH₂CF₃ |
| A-259 | CH(CH₃)₂ | CH(CH₃)₂ | CH₃ | CH₂CH═CH₂ |
| A-260 | CH(CH₃)₂ | CH(CH₃)₂ | CH₃ | C₆H₅ |
| A-261 | CH(CH₃)₂ | CH(CH₃)₂ | CH₃ | CH₂C₆H₅ |
| A-262 | CH(CH₃)₂ | CH(CH₃)₂ | C₂H₅ | H |
| A-263 | CH(CH₃)₂ | CH(CH₃)₂ | C₂H₅ | CH₃ |
| A-264 | CH(CH₃)₂ | CH(CH₃)₂ | C₂H₅ | C₂H₅ |
| A-265 | CH(CH₃)₂ | CH(CH₃)₂ | C₂H₅ | CH(CH₃)₂ |
| A-266 | CH(CH₃)₂ | CH(CH₃)₂ | C₂H₅ | ^{c}(CH₂)₃ |
| A-267 | CH(CH₃)₂ | CH(CH₃)₂ | C₂H₅ | CH₂CF₃ |
| A-268 | CH(CH₃)₂ | CH(CH₃)₂ | C₂H₅ | CH₂CH═CH₂ |
| A-269 | CH(CH₃)₂ | CH(CH₃)₂ | C₂H₅ | C₆H₅ |
| A-270 | CH(CH₃)₂ | CH(CH₃)₂ | C₂H₅ | CH₂C₆H₅ |

^{c}(CH₂)₃ = cyclopropyl

The compounds of the formula (I) can be prepared by the standard methods of organic chemistry, e.g. by the methods described hereinafter in schemes 1 to 9 and in the synthesis descriptions of the working examples. The substituents, variables and indices in schemes 1 to 9 are as defined above for formula (I), if not otherwise specified.

The compounds of formula (I) can be prepared as shown in the Scheme 1 below.

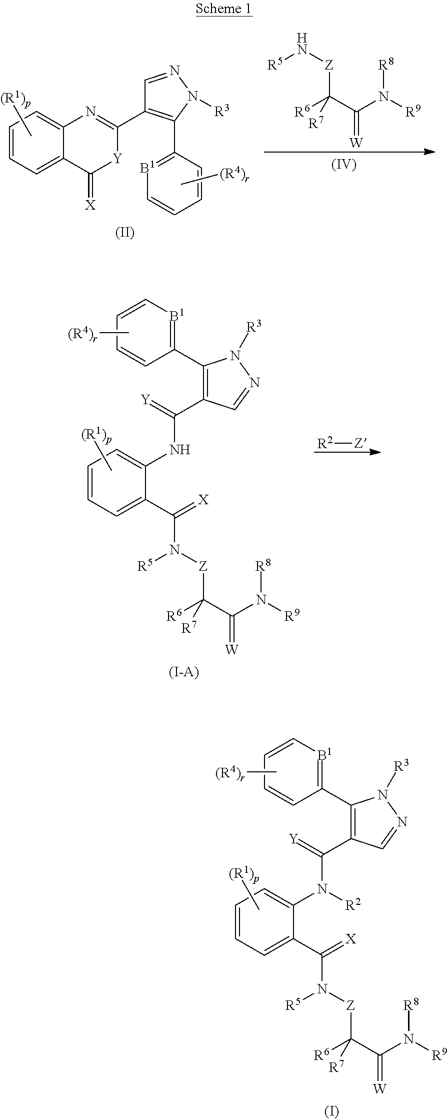

Compounds of formula (II) are reacted with compounds of formula (IV) to give compounds of formula (I-A). The reaction is suitably carried out in a polar or apolar aprotic solvent such as N,N-dimethylformamide, tetrahydrofuran, dioxane, acetonitrile, dimethylsulfoxide or pyridine, or mixtures of these solvents, in a temperature range between 0° C. and 100° C., preferably between 20° C. and 90° C. For converting compounds of formula (I-A) in which R² is H into compounds (I) in which R² is not H, compounds of formula (I-A) can be reacted with compounds of formula R²—Z′, wherein R² is not H and Z′ is a leaving group, such as for example a bromine, chlorine or iodine atom or a tosylate, mesylate or triflate, to give compounds of formula (I). The reaction is suitably carried out in the presence of a base such as sodium hydride or potassium hydride, suitably in a polar aprotic solvent such as N,N-dimethylformamide, tetrahydrofuran, dioxane, acetonitrile, dimethylsulfoxide or pyridine, or mixtures of these solvents, in a temperature range between 0° C. and 100° C. Other preparation methods for compounds of formula I may also be adapted from analogous reactions, as for example described in WO 01/70671.

Benoxzazin(thi)ones and benzothiazin(thi)ones of formula (II) are available via known methods, e.g. via coupling of either an anthranilic acid or an isatoic anhydride with an acid chloride. For references to the synthesis and chemistry of benzazinones see Jacobsen et al, Bioorganic and Medicinal Chemistry, 2000, 8, 2095-2103 and references cited therein. See also Coppola, J. Heterocyclic Chemistry, 1999, 36, 563-588. The benzazin(thi)ones of formula (II) can also be prepared according to the procedures described in WO 04/046129 or WO 04/011447 as well as according to references cited therein and suitable modifications thereof.

Compounds of formula (IV), in which Z is an oxygen atom, one of either R⁶ or R⁷ is H and R⁵ to R⁹ are otherwise as described as for formula (I), can be obtained as shown in Scheme 2 below.

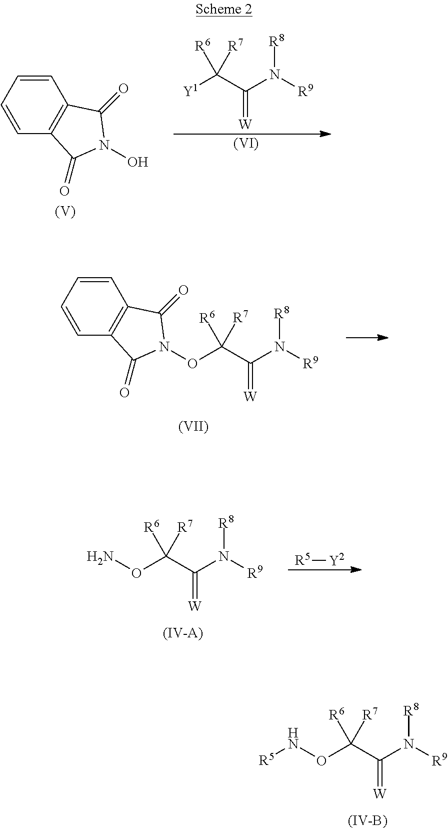

Reaction of N-hydroxyphthalamide (V) with compounds of formula (VI), in which Y¹ is a chloro, bromo, iodo or hydroxyl radical, gives compounds of formula (VII). The reaction, excluding the case where Y¹ is hydroxyl, is carried out in the presence of a base such as a group I metal carbonate, a trialkylamine or a group I metal hydride in the presence of an aprotic polar solvent such as N-methylpyrrolidin-2-one, N,N-dimethylformamide or tetrahydrofuran between 25° C. and 180° C. In the case where Y′ is hydroxyl, the reaction may proceed by Mitsonobu reaction in the presence of a suitable trialkyl or triaryl phosphine reagent and an N,N′-dialkylazodicarboxylate reagent, in analogy to conditions described in Organic Letters, 2009, 11(9), 2019-2022 or Synthesis, (4), 655-659, and references therein. Cleavage of the phthalamide protecting group in the compound of formula (VII) to give the compound of formula (IV-A), which is a special case of compound of formula (IV) where Z is oxygen, R⁵ is hydrogen and either one of R⁶ and R⁷ are hydrogen, is carried out in the presence of hydrazine or methylhydrazine in a polar protic solvent such as methanol or ethanol between 0° C. and 80° C. The compound of formula (IV-B), which is a special case of formula (IV) wherein Z is oxygen and either one of R⁶ and R⁷ are hydrogen, is synthesized starting from compounds of formula (IV-A) in the presence of an alkylating group of formula R⁵—Y², a weak base such as a trialkylamine, sodium carbonate, or potassium carbonate and an aprotic polar solvent such as dichloromethane, chloroform, acetonitrile, N,N-dimethylformamide, N,N-dimethyl-acetamide or N-methylpyrrolidin-2-one, between 0° C. and 150° C. R⁵ is as defined for formula (I) and Y² is leaving group, such as a chloro, bromo or iodo radical. Compounds of formula (IV-A) and (IV-B) can be reacted analogously to compounds of formula (IV) with compounds of formula (II) to give sequentially the compounds of formula (I-A) and (I), as described in Scheme 1, above.

The compounds of formula (VI) can be obtained by following standard amide bond formation of the respective amine R⁸R⁹NH with the corresponding acid chloride (Y)(R⁶)(R⁷)CCOCl.

Compounds of formula (IV-A) and (IV-B) wherein R⁶ and R⁷ are both alkyl groups can be prepared as follows:

Reaction of a compound (VIII) with compound (IX) to give compound (X) can be effected by following the literature procedure in J. Medicinal Chemistry, 2008, 41(15), 4601-4608 (Supporting Information, Compound I-d), see also Scheme 3 below.

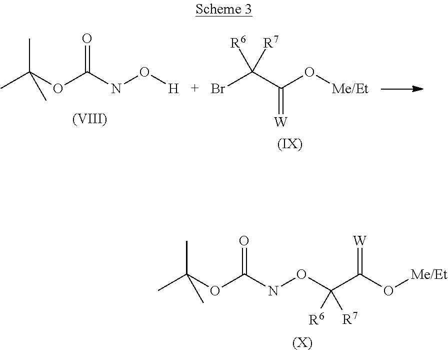

wherein “Me” means methyl and “Et” means ethyl.

As shown in Scheme 4 below, compound (X) can be hydrolysed to the free acid (XI) using standard ester hydrolysis conditions, such as using metal hydroxides in a suitable polar protic solvent or solvent mixture. Treating the free acid with a suitable amine HNR⁸R⁹ under standard amide coupling conditions delivers the amide (XII).

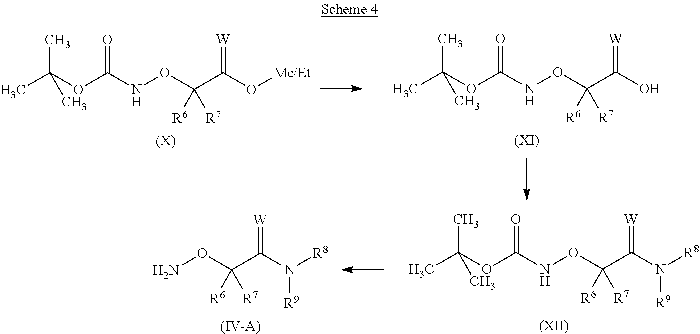

wherein “Me” means methyl and “Et” means ethyl.

Under acidic conditions, tert-butyl carbamates are known to decompose to the free amine, as described in Greene, T. W.; Wutz, P. G. M., Protective Groups in Organic Synthesis, Wiley, Fourth Ed. Thus compounds of formula (IV-A), wherein neither R⁶ nor R⁷ are hydrogen atoms and the substituents are otherwise as defined for formula (I), can be prepared by reaction of compounds of formula (XII) with concentrated mineral acid or trifluoroacetic acid in an aprotic organic solvent at temperatures between 0° C. and 50° C.

In the special case where in compound (IV) R⁵ is hydrogen and Z is sulfur, these intermediates may be prepared as follows and reacted further as with compound (II) to give the compounds of formula (I) in which R⁵ is H and Z is sulfur:

The compound of formula (II) is reacted with a compound PH₃C—S—NH₂, wherein Ph is phenyl, which is commercially available (see Scheme 5 below).

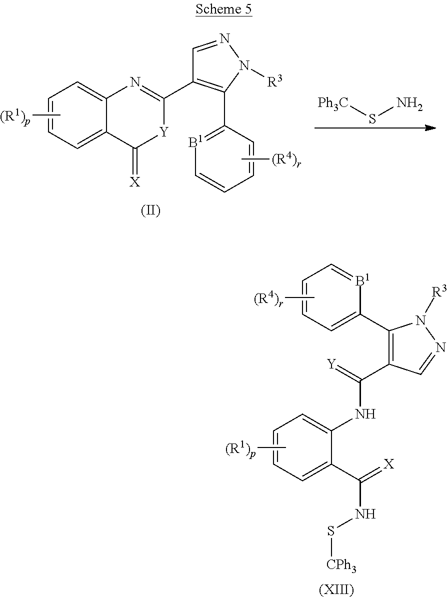

wherein “Ph” means phenyl.

The reaction is carried out in an aprotic solvent such as N,N-dimethylformamide, tetrahydrofuran, dioxane, acetonitrile, dimethylsulfoxide or pyridine, or mixtures of these solvents, in a temperature range between 0° C. and 100° C., preferably between 20° C. and 90° C.

Compound (XIII) can be hydrolyzed to compound (XIV) under reductive conditions such as using a trialkylsilane in the presence of trifluoroacetic acid (e.g. in Chemistry, A European Journal, 2003, 9(15), 3683-91), see also Scheme 6 below.

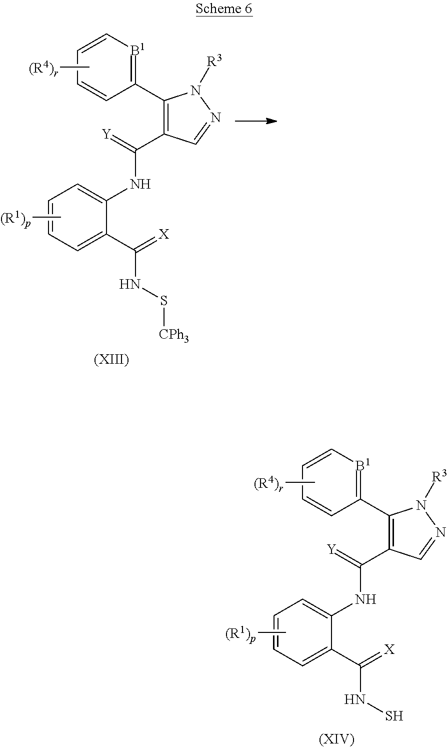

wherein “Ph” means phenyl.

As can be seen from Scheme 7 below, compound (XIV) can be alkylated with a compound of formula (XV), wherein Y¹ is a leaving group, using a suitable base, such as a metal hydroxide, a metal hydride, a metal carbonate or a metal alkoxide in a suitable aprotic solvent such as dimethylsulfoxide, acetonitrile, N-methyl-pyrrole, tetrahydrofuran, dioxane, N,N-dimethylformamide or a suitable mixture of the above solvents, yielding a compound (I-B), wherein Z is S and R² and R⁵ are H.

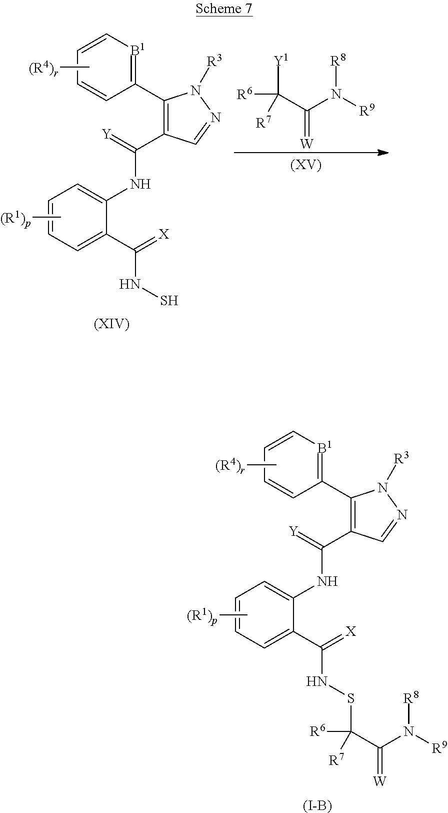

In case where in compound (IV) R⁵ is hydrogen and Z is NR^{12a}, these intermediates may be prepared as follows and reacted further as with compound (IV) to give the compounds of formula (I) in which R⁵ is H and Z is NR^{12a}:

As can be seen in Scheme 8 below, the compound (XV) can be treated with aqueous hydrazine using a suitable solvent, such as dimethylsulfoxide, acetonitrile, N-methyl-pyrrole, tetrahydrofuran, dioxane, N,N-dimethylformamide or a suitable mixture of the above solvents at a temperature range between 0° C. and 100° C., preferably between 20° C. and 90° C. to give the compound (XVI).

Compound (II) can be ring-opened with compound (XVI) to give compound (I-C) using a suitable solvent such as N,N-dimethylformamide, tetrahydrofuran, dioxane, acetonitrile, dimethylsulfoxide or pyridine, or mixtures of these solvents, in a temperature range between 0° C. and 100° C., preferably between 20° C. and 90° C.

Compound (I-C) can be treated with compounds of the formula R^{12a}—Y², wherein R^{12a} and Y² are as defined above with or without the presence of a suitable base such as a metal hydroxide, alkoxide, amide, carbonate or hydride, or a trialkylamine in a suitable solvent to give a compound I-D, wherein Z is NR^{12a} and R² and R⁵ are H.

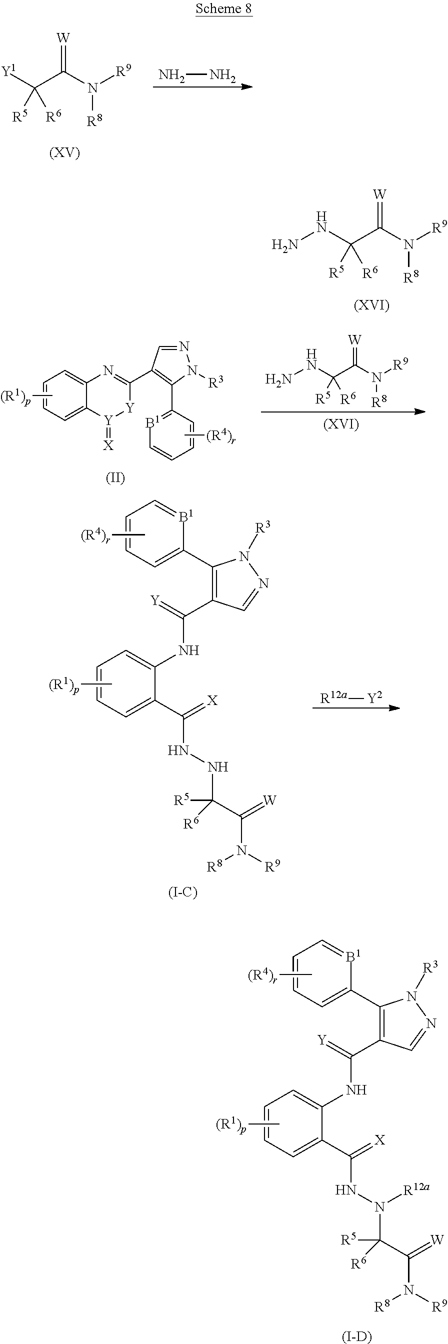

Alternatively, compounds of formula (I) can also be prepared as shown in scheme 9 below by reaction of a compound of formula (III) with a compound of formula (IV).

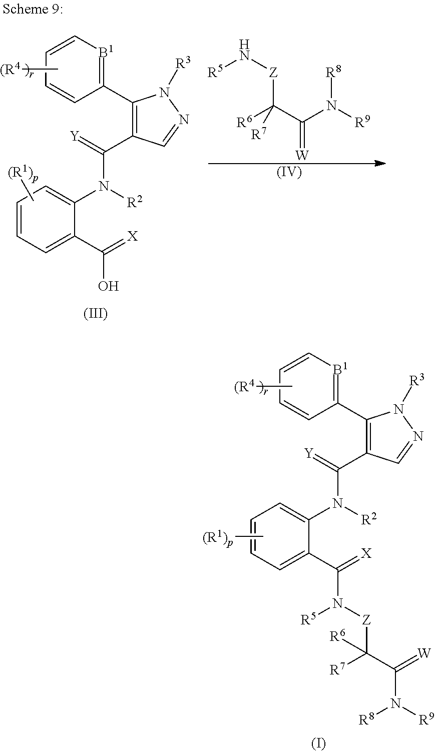

As a rule, the compounds of formula (I) including their stereoisomers, salts, tautomers and N-oxides, and their precursors in the synthesis process can be prepared by the methods described above. If individual compounds can not be prepared via the above described routes, they can be prepared by derivatization of other compounds (I) or the respective precursor or by customary modifications of the synthesis routes described. For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

Due to their excellent activity, the compounds of the present invention may be used for controlling invertebrate pests.

Accordingly, the present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of the present invention or a composition as defined above.

Preferably, the method of the invention serves for protecting plant propagation material (such as seed) and the plant which grows therefrom from invertebrate pest attack or infestation and comprises treating the plant propagation material (such as seed) with a pesticidally effective amount of a compound of the present invention as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

In the sense of the present invention, “invertebrate pests” are preferably selected from arthropods and nematodes, more preferably from harmful insects, arachnids and nematodes, and even more preferably from insects, acarids and nematodes. In the sense of the present invention, “invertebrate pests” are most preferably insects.

The invention further provides an agricultural composition for combating invertebrate pests, which comprises such an amount of at least one compound according to the invention and at least one inert liquid and/or solid agronomically acceptable carrier that has a pesticidal action and, if desired, at least one surfactant.

Such a composition may comprise a single active compound of the present invention or a mixture of several active compounds of the present invention. The composition according to the present invention may comprise an individual isomer or mixtures of isomers or a salt as well as individual tautomers or mixtures of tautomers.

The compounds of the present invention, including their salts, stereoisomers and tautomers, are in particular suitable for efficiently controlling arthropodal pests such as arachnids, myriapedes and insects as well as nematodes. They are especially suitable for efficiently combating or controlling the following pests:

Insects from the order of the lepidopterans (Lepidoptera), for example Agrotis ipsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Pieris rapae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria;
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;
thrips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,
termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus;
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis;
bugs, aphids, leafhoppers, whiteflies, scale insects, cicadas (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus hesperus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nephotettix virescens, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus;
ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;
crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina;
arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa;
fleas (Siphonaptera), e.g. Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica,
centipedes (Chilopoda), e.g. Scutigera coleoptrata,
millipedes (Diplopoda), e.g. Narceus spp.,
Earwigs (Dermaptera), e.g. forficula auricularia,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
Collembola (springtails), e.g. Onychiurus ssp.

The compounds of the present invention, including their salts, stereoisomers and tautomers, are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the present invention, including their salts, stereoisomers and tautomers, are particularly useful for controlling insects, preferably chewing and biting and piercing and sucking insects such as insects from the genera Lepidoptera, Coleoptera and Hemiptera, in particular Lepidoptera, Coleoptera and true bugs.

The compounds of the present invention, including their salts, stereoisomers and tautomers, are moreover useful for controlling insects of the orders Thysanoptera, Diptera (especially flies, mosquitoes), Hymenoptera (especially ants) and Isoptera (especially termites).

The compounds of the present invention, including their salts, stereoisomers and tautomers, are particularly useful for controlling insects of the orders Lepidoptera and Coleoptera.

The compounds of the present invention can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the present invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones [N-methyl-pyrrolidone (NMP), N-octylpyrrolidone (NOP)], acetates (glycol diacetate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are surface-active compounds, such as anionic, cationic, nonionic and am-photeric surfactants, block polymers, polyelectrolytes, and mixtures thereof.

Such surfactants can be used as emulsifier, dispersant, solubilizer, wetter, penetration enhancer, protective col-loid, or adjuvant. Examples of surfactants are listed in McCutcheon's, Vol. 1: Emulsifiers & De-tergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of con-densed naphthalenes, sulfonates of dodecyl- and tridecylbenzenes, sulfonates of naphthalenes and alkyl [symbol] naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Exam-ples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkyl-polyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Also anti-freezing agents, such as glycerin, ethylene glycol or propylene glycol, and bactericides, such as bronopol and isothiazolinone derivatives such as alkylisothiazolinones and benzisothiazolinones, can be added to the formulation.

Suitable antifoaming agents are for example antifoaming agents based on silicon or magnesium stearate.

Suitable preservatives are for example dichlorophen und benzyl alcohol hemiformal

Suitable thickeners are compounds which confer a pseudoplastic flow behavior to the formulation, i.e. high viscosity at rest and low viscosity in the agitated stage. Mention may be made, in this context, for example, of commercial thickeners based on polysaccharides, such as Xanthan Gum® (Kelzan® from Kelco), Rhodopol®23 (Rhone Poulenc) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations, i.e. the compositions according to the invention, comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of the present invention can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

The following are examples of formulations:

1. Products for dilution with water. For seed treatment purposes, such products may be applied to the seed diluted or undiluted.

### A) Water-Soluble Concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active compound is obtained.

### B) Dispersible Concentrates (DC)

20 parts by weight of the active compound is dissolved in 70 parts by weight of cyclohexanone with addition of 10 parts by weight of a dispersant, for example polyvinylpyrrolidone. Dilution with water gives a dispersion, whereby a formulation with 20% (w/w) of active compounds is obtained.

### C) Emulsifiable Concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

### D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

### E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

### F) Water-Dispersible Granules and Water-Soluble Granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

### G) Water-Dispersible Powders and Water-Soluble Powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

### H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.

2. Products to be applied undiluted for foliar applications. For seed treatment purposes, such products may be applied to the seed diluted or undiluted.

### I) Dustable Powders (DP, DS)

5 parts by weight of the active compound are ground finely and mixed intimately with 95 parts by weight of finely divided kaolin. This gives a dustable product having 5% (w/w) of active compound.

### J) Granules (GR, FG, GG, MG)

0.5 part by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

### K) ULV Solutions (UL)

10 parts by weight of the active compound is dissolved in 90 parts by weight of an organic solvent, for example xylene. This gives a product having 10% (w/w) of active compound, which is applied undiluted for foliar use.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.001 to 1%.

The active ingredients may also be used successfully in the ultra-low-volume process (ULV), it being possible to apply formulations comprising over 95% by weight of active ingredient, or even to apply the active ingredient without additives.

In the methods and uses of this invention, the compounds according to the invention may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following list M of pesticides together with which the compounds according to the invention can be used and with which potential synergistic effects might be produced, is intended to illustrate the possible combinations, but not to impose any limitation:

M.1. Organo(thio)phosphate compounds: acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
M.2. Carbamate compounds: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;
M.3. Pyrethroid compounds: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zetacypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, metofluthrin, permethrin, phenothrin, prallethrin, profluthrin, pyrethrin (pyrethrum), resmethrin, silafluofen, tefluthrin, tetramethrin, tralomethrin, transfluthrin;
M.4. Juvenile hormone mimics: hydroprene, kinoprene, methoprene, fenoxycarb, pyriproxyfen;
M.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), spinetoram (allosteric agonist), thiacloprid, thiocyclam, thiosultap-sodium and AKD1022.
M.6. GABA gated chloride channel antagonist compounds: chlordane, endosulfan, gamma-HCH (lindane); ethiprole, fipronil, pyrafluprole, pyriprole M.7. Chloride channel activators: abamectin, emamectin benzoate, milbemectin, lepimectin;
M.8. METI I compounds: fenazaquin, fenpyroximate, pyrimidifen, pyridaben, tebufenpyrad, tolfenpyrad, flufenerim, rotenone;
M.9. METI II and III compounds: acequinocyl, fluacyprim, hydramethylnon;
M.10. Uncouplers of oxidative phosphorylation: chlorfenapyr, DNOC;
M.11. Inhibitors of oxidative phosphorylation: azocyclotin, cyhexatin, diafenthiuron, fenbutatin oxide, propargite, tetradifon;
M.12. Moulting disruptors: cyromazine, chromafenozide, halofenozide, methoxyfenozide, tebufenozide;
M.13. Synergists: piperonyl butoxide, tribufos;
M.14. Sodium channel blocker compounds: indoxacarb, metaflumizone;
M.15. Fumigants: methyl bromide, chloropicrin sulfuryl fluoride;
M.16. Selective feeding blockers: crylotie, pymetrozine, flonicamid;
M.17. Mite growth inhibitors: clofentezine, hexythiazox, etoxazole;

M. 18. Chitin synthesis inhibitors: buprofezin, bistrifluron, chlorfluazuron, diflubenzuron, flucycloxuron, flufenoxuron, hexaflumuron, lufenuron, novaluron, noviflumuron, teflubenzuron, triflumuron;

M.19. Lipid biosynthesis inhibitors: spirodiclofen, spiromesifen, spirotetramat;
M.20. Octapaminergic agonsits: amitraz;
M.21. Ryanodine receptor modulators: flubendiamide and the phtalamid compound (R)-, (S)-3-Chlor-N1-{2-methyl-4-[1,2,2,2-tetrafluor-1-(trifluormethyl)ethyl]phenyl}-N2-(1-methyl-2-methylsulfonylethyl)phthalamid (M21.1)
M.22. Isoxazoline compounds: 4-[5-(3,5-Dichloro-phenyl)-5-trifluoromethyl-4,5-dihydro-isoxazol-3-yl]-2-methyl-N-pyridin-2-ylmethyl-benzamide (M22.1), 4-[5-(3,5-Dichloro-phenyl)-5-trifluoromethyl-4,5-dihydro-isoxazol-3-yl]-2-methyl-N-(2,2,2-trifluoro-ethyl)-benzamide (M22.2), 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide (M22.3), 4-[5-(3,5-Dichloro-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.4), 4-[5-(3,5-Dichlorophenyl)-5-trifluoromethyl-4,5-dihydro-isoxazol-3-yl]-N-[(methoxyimino)methyl]-2-methylbenzamide (M22.5) 4-[5-(3-Chloro-5-trifluoromethyl-phenyl)-5-trifluoromethyl-4,5-dihydro-isoxazol-3-yl]-2-methyl-N-[(2,2,2-trifluoro-ethylcarbamoyl)-methyl]-benzamide (M22.6), 4-[5-(3-Chloro-5-trifluoromethyl-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.7) and 5-[5-(3,5-Dichloro-4-fluoro-phenyl)-5-trifluoromethyl-4,5-dihydro-isoxazol-3-yl]-2-[1,2,4]triazol-1-yl-benzonitrile (M22.8);
M.23. Anthranilamide compounds: chloranthraniliprole, cyantraniliprole, 5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (M23.1), 5-Bromo-2-(3-chloro-pyridin-2-yl)-2H-pyrazole-3-carboxylic acid [2-chloro-4-cyano-6-(1-cyclopropyl-ethylcarbamoyl)-phenyl]-amide (M23.2), 5-Bromo-2-(3-chloro-pyridin-2-yl)-2H-pyrazole-3-carboxylic acid [2-bromo-4-cyano-6-(1-cyclopropyl-ethylcarbamoyl)-phenyl]-amide (M23.3), 5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide (M23.4), 5-Bromo-2-(3-chloro-pyridin-2-yl)-2H-pyrazole-3-carboxylic acid [2,4-dichloro-6-(1-cyclopropyl-ethylcarbamoyl)-phenyl]-amide (M23.5), 5-Bromo-2-(3-chloro-pyridin-2-yl)-2H-pyrazole-3-carboxylic acid [4-chloro-2-(1-cyclopropyl-ethylcarbamoyl)-6-methyl-phenyl]-amide (M23.6), N′-(2-{[5-Bromo-2-(3-chloro-pyridin-2-yl)-2H-pyrazole-3-carbonyl]-amino}-5-chloro-3-methyl-benzoyl)-hydrazinecarboxylic acid methyl ester (M23.7), N′-(2-{[5-Bromo-2-(3-chloro-pyridin-2-yl)-2H-pyrazole-3-carbonyl]-amino}-5-chloro-3-methyl-benzoyl)-N′-methyl-hydrazinecarboxylic acid methyl ester (M23.8), N′-(2-{[5-Bromo-2-(3-chloro-pyridin-2-yl)-2H-pyrazole-3-carbonyl]-amino}-5-chloro-3-methyl-benzoyl)-N,N′-dimethyl-hydrazinecarboxylic acid methyl ester (M23.9), N′-(3,5-Dibromo-2-{[5-bromo-2-(3-chloro-pyridin-2-yl)-2H-pyrazole-3-carbonyl]-amino}-benzoyl)-hydrazinecarboxylic acid methyl ester (M23.10), N′-(3,5-Dibromo-2-{[5-bromo-2-(3-chloro-pyridin-2-yl)-2H-pyrazole-3-carbonyl]-amino}-benzoyl)-N′-methyl-hydrazinecarboxylic acid methyl ester (M23.11) and N′-(3,5-Dibromo-2-{[5-bromo-2-(3-chloro-pyridin-2-yl)-2H-pyrazole-3-carbonyl]-amino}-benzoyl)-N,N′-dimethyl-hydrazinecarboxylic acid methyl ester (M23.12);
M.24. Malononitrile compounds: 2-(2,2,3,3,4,4,5,5-octafluoropentyl)-2-(3,3,3-trifluoropropyl)malononitrile (CF₂HCF₂CF₂CF₂CH₂C(CN)₂CH₂CH₂CF₃) (M24.1) and 2-(2,2,3,3,4,4,5,5-octafluoropentyl)-2-(3,3,4,4,4-pentafluorobutyl)-malonodinitrile (CF₂HCF₂CF₂—CF₂CH₂C(CN)₂CH₂CH₂CF₂CF₃) (M24.2);
M.25. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;
M.26. Aminofuranone compounds:
4-{[(6-Bromopyrid-3-yl)methyl](2-fluoroethyl)amino}furan-2(5H)-on (M26.1), 4-{[(6-Fluoropyrid-3-yl)methyl](2,2-difluoroethyl)amino}furan-2(5H)-on (M26.2), 4-{[(2-Chloro1,3-thiazolo-5-yl)methyl](2-fluoroethyl)amino}furan-2(5H)-on (M26.3), 4-{[(6-Chloropyrid-3-yl)methyl](2-fluoroethyl)amino}furan-2(5H)-on (M26.4), 4-{[(6-Chloropyrid-3-yl)methyl](2,2-difluoroethyl)amino}furan-2(5H)-on (M26.5), 4-{[(6-Chloro-5-fluoropyrid-3-yl)methyl](methyl)amino}furan-2(5H)-on (M26.6), 4-{[(5,6-Dichloropyrid-3-yl)methyl](2-fluoroethyl)amino}furan-2(5H)-on (M26.7), 4-{[(6-Chloro-5-fluoropyrid-3-yl)methyl](cyclopropyl)amino}furan-2(5H)-on (M26.8), 4-{[(6-Chloropyrid-3-yl)methyl](cyclopropyl)amino}furan-2(5H)-on (M26.9) and 4-{[(6-Chloropyrid-3-yl)methyl](methyl)amino}furan-2(5H)-on (M26.10);
M.27. Various compounds: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic, sulfoxaflor, N—R′-2,2-dihalo-1-R″cyclo-propanecarboxamide-2-(2,6-dichloro-α,α,α,-trifluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, 4-But-2-ynyloxy-6-(3,5-dimethyl-piperidin-1-yl)-2-fluoro-pyrimidine (M27.1), Cyclopropaneacetic acid, 1,1′-[(3S,4R,4aR,6S,6aS,12R,12aS,12bS)-4-[[(2-cyclopropyl-acetyl)oxy]methyl]-1,3,4,4a,5,6,6a,12,12a,12b-decahydro-12-hydroxy-4,6a,12b-trimethyl-11-oxo-9-(3-pyridinyl)-2H, 11H-naphtho[2,1-b]pyrano[3,4-e]pyran-3,6-diyl]ester (M27.2) and 8-(2-Cyclopropylmethoxy-4-trifluoromethyl-phenoxy)-3-(6-trifluoromethyl-pyridazin-3-yl)-3-aza-bicyclo[3.2.1]octane (M27.3).

The commercially available compounds of the group M may be found in The Pesticide Manual, 13th Edition, British Crop Protection Council (2003) among other publications. Paraoxon and their preparation have been described in Farm Chemicals Handbook, Volume 88, Meister Publishing Company, 2001. Flupyrazofos has been described in Pesticide Science 54, 1988, p. 237-243 and in U.S. Pat. No. 4,822,779.-AKD 1022 and its preparation have been described in U.S. Pat. No. 6,300,348.-M21.1 is known from WO 2007/101540. Isoxazolines M22.1 to M22.8 have been described in e.g. WO 2005/085216, WO 2007/079162, WO 2007/026965, WO 2009/126668 and WO 2009/051956. Anthranilamides M23.1 to M23.6 have been described in WO 2008/72743 and WO 200872783, those M23.7 to M23.12 in WO 2007/043677. Malononitriles M24.1 and M24.2 have been described in WO 02/089579, WO 02/090320, WO 02/090321, WO 04/006677, WO 05/068423, WO 05/068432 and WO 05/063694. Aminofuranones M26.1 to M26.10 have been described e.g. in WO 2007/115644. Alkynylether M27.1 is described e.g. in JP 2006131529. Organic sulfur compounds have been described in WO 2007060839. Pyripyropene derivative M27.2 has been described in WO 2008/66153 and WO 2008/108491. Pyridazin M27.3 has been described in JP 2008/115155.

The following list F of active substances, in conjunction with which the compounds according to the invention can be used, is intended to illustrate the possible combinations but does not limit them:

F.I) Respiration inhibitors
F.I-1) Inhibitors of complex III at Qo site (e.g. strobilurins)
strobilurins: azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, triclopyricarb/chlorodincarb, trifloxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methyl-acetamide; oxazolidinediones and imidazolinones: famoxadone, fenamidone;
F.I-2) Inhibitors of complex II (e.g. carboxamides):
carboxanilides: benodanil, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide, N-(4′-trifluoromethylthiobiphenyl-2-yl)-3 difluoromethyl-1-methyl-1H pyrazole-4-carboxamide and N-(2-(1,3,3-trimethyl-butyl)-phenyl)-1,3-dimethyl-5 fluoro-1H-pyrazole-4 carboxamide;
F.I-3) Inhibitors of COMPLEX III at Qi site: cyazofamid, amisulbrom;
F.I-4) Other respiration inhibitors (complex I, uncouplers)
diflumetorim; tecnazen; ferimzone; ametoctradin; silthiofam; nitrophenyl derivates: binapacryl, dinobuton, dinocap, fluazinam, nitrthal-isopropyl, organometal compounds: fentin salts, such as fentin-acetate, fentin chloride or fentin hydroxide;
F.II) Sterol biosynthesis inhibitors (SBI fungicides)
F.II-1) C14 DEMETHYLASE INHIBITORS (DMI fungicides, e.g. triazoles, imidazoles)
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole; imidazoles: imazalil, pefurazoate, oxpoconazole, prochloraz, triflumizole; pyrimidines, pyridines and piperazines: fenarimol, nuarimol, pyrifenox, triforine;
F.II-2) Delta14-Reductase Inhibitors (Amines, e.g. Morpholines, Piperidines)
morpholines: aldimorph, dodemorph, dodemorph-acetate, fenpropimorph, tridemorph; piperidines: fenpropidin, piperalin; spiroketalamines: spiroxamine;
F.II-3) Inhibitors of 3-keto reductase: hydroxyanilides: fenhexamid;

F.III) Nucleic acid synthesis inhibitors

F.III-1) RNA, DNA synthesis
phenylamides or acyl amino acid fungicides: benalaxyl, benalaxyl-M, kiralaxyl, metalaxyl, metalaxyl-M (mefenoxam), ofurace, oxadixyl; isoxazoles and iosothiazolones: hymexazole, octhilinone;
F.III-2) DNA topisomerase inhibitors: oxolinic acid;
F.III-3) Nucleotide metabolism (e.g. adenosin-deaminase) hydroxy(2-amino)-pyrimidines: bupirimate;
F.IV) Inhibitors of cell division and or cytoskeleton
F.IV-1) Tubulin inhibitors: benzimidazoles and thiophanates: benomyl, carbendazim, fuberidazole, thiabendazole, thiophanate-methyl;
triazolopyrimidines: 5-chloro-7 (4-methylpiperidin-1-yl)-6-(2,4,6-trifluorophenyl)-[1,2,4]triazolo[1,5 a]pyrimidine
F.IV-2) Other cell division inhibitors benzamides and phenyl acetamides: diethofencarb, ethaboxam, pencycuron, fluopicolide, zoxamide;
F.IV-3) Actin inhibitors: benzophenones: metrafenone;
F.V) Inhibitors of amino acid and protein synthesis
F.V-1) Mmethionine synthesis inhibitors (anilino-pyrimidines) anilino-pyrimidines: cyprodinil, mepanipyrim, nitrapyrin, pyrimethanil;
F.V-2) Protein synthesis inhibitors (anilino-pyrimidines)
antibiotics: blasticidin-S, kasugamycin, kasugamycin hydrochloride-hydrate, mildiomycin, streptomycin, oxytetracyclin, polyoxine, validamycin A;
F.VI) Signal transduction inhibitors
F.VI-1) MAP/Histidine kinase inhibitors (e.g. anilino-pyrimidines) dicarboximides: fluoroimid, iprodione, procymidone, vinclozolin; phenylpyrroles: fenpiclonil, fludioxonil;
F.VI-2) G protein inhibitors: quinolines: quinoxyfen;
F.VII) Lipid and membrane synthesis inhibitors
F.VII-1) Phospholipid biosynthesis inhibitors
organophosphorus compounds: edifenphos, iprobenfos, pyrazophos; dithiolanes: isoprothiolane;
F.VII-2) Lipid peroxidation
aromatic hydrocarbons: dicloran, quintozene, tecnazene, tolclofos-methyl, biphenyl, chloroneb, etridiazole;
F.VII-3) Carboxyl acid amides (CAA fungicides)
cinnamic or mandelic acid amides: dimethomorph, flumorph, mandiproamid, pyrimorph; valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;
F.VII-4) Compounds affecting cell membrane permeability and fatty acids carbamates: propamocarb, propamocarb-hydrochlorid
F.VIII) Inhibitors with Multi Site Action
F.VIII-1) Inorganic active substances: Bordeaux mixture, copper acetate, copper hydroxide, copper oxychloride, basic copper sulfate, sulfur;
F.VIII-2) Thio- and dithiocarbamates: ferbam, mancozeb, maneb, metam, methasulphocarb, metiram, propineb, thiram, zineb, ziram;
F.VIII-3) Organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;
F.VIII-4) Guanidines: guanidine, dodine, dodine free base, guazatine, guazatineacetate, iminoctadine, iminoctadine-triacetate, iminoctadine-tris(albesilate);
F.VIII-5) Ahtraquinones: dithianon;
F.IX) Cell wall synthesis inhibitors
F.IX-1) Inhibitors of glucan synthesis: validamycin, polyoxin B;
F.IX-2) Melanin synthesis inhibitors: pyroquilon, tricyclazole, carpropamide, dicyclomet, fenoxanil;
F.X) Plant defence inducers
F.X-1) Salicylic acid pathway: acibenzolar-S-methyl;
F.X-2) Others: probenazole, isotianil, tiadinil, prohexadione-calcium; phosphonates: fosetyl, fosetyl-aluminum, phosphorous acid and its salts;
F.XI) Unknown mode of action:
bronopol, chinomethionat, cyflufenamid, cymoxanil, dazomet, debacarb, diclomezine, difenzoquat, difenzoquat-methylsulfate, diphenylamin, flumetover, flusulfamide, flutianil, methasulfocarb, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide;
F.XI) Growth regulators:
abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6 benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;
F.XII) Biological control agents
antifungal biocontrol agents: Bacillus subtilis strain with NRRL No. B-21661 (e.g. RHAPSODY®, SERENADE® MAX and SERENADE® ASO from AgraQuest, Inc., USA.), Bacillus pumilus strain with NRRL No. B-30087 (e.g. SONATA® and BALLAD® Plus from AgraQuest, Inc., USA), Ulocladium oudemansii (e.g. the product BOTRYZEN from BotriZen Ltd., New Zealand), Chitosan (e.g. ARMOUR-ZEN from BotriZen Ltd., New Zealand).

The invention also relates to a composition containing one or more, preferably one, individualized compound(s) I of the invention and one or more, preferably one, two or three, in particular one, pesticide(s) selected from the above list M and/or F.

The composition according to the invention may be a physical mixture of the at least one compound I of the invention and the at least one pesticide selected from the above list M and/or F. Accordingly, the invention also provides a mixture comprising one or more, preferably one, compound(s) I of the invention and one or more, preferably one, two or three, in particular one, pesticide(s) selected from the above list M and/or F. However, the composition may also be any combination of at least one compound I of the invention with at least one pesticide selected from the above list M and/or F, it not being required for the compounds to be present together in the same formulation.

An example of a composition according to the invention in which the at least one compound I of the invention and the at least one pesticide selected from the above list M and/or F are not present together in the same formulation is a combipack. In a combipack, two or more components of a combipack are packaged separately, i.e., not jointly pre-formulated. As such, combipacks include one or more separate containers such as vials, cans, bottles, pouches, bags or canisters, each container containing a separate component for an agrochemical composition. One example is a two-component combipack. Accordingly the present invention also relates to a two-component combipack, comprising a first component which in turn comprises at least one compound A, a liquid or solid carrier and, if appropriate, at least one surfactant and/or at least one customary auxiliary, and a second component which in turn comprises at least one compound B, a liquid or solid carrier and, if appropriate, at least one surfactant and/or at least one customary auxiliary. More details, e.g. as to suitable liquid and solid carriers, surfactants and customary auxiliaries are described below.

The invertebrate pest (also referred to as “animal pest”), i.e. the insects, arachnids and nematodes, the plant, soil or water in which the plant is growing or may grow can be contacted with the compounds of the present invention or composition(s) comprising them by any application method known in the art. As such, “contacting” includes both direct contact (applying the compounds/compositions directly on the invertebrate pest or plant—typically to the foliage, stem or roots of the plant) and indirect contact (applying the compounds/compositions to the locus of the invertebrate pest or plant). The compounds of the present invention or the pesticidal compositions comprising them may be used to protect growing plants and crops from attack or infestation by animal pests, especially insects, acaridae or arachnids by contacting the plant/crop with a pesticidally effective amount of compounds of the present invention. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with an insecticidally effective amount of the active compounds. The application can be carried out both before and after the infection of the plants, plant propagation materials, such as seeds, soil, surfaces, materials or rooms by the insects.

Moreover, invertebrate pests may be controlled by contacting the target pest, its food supply, habitat, breeding ground or its locus with a pesticidally effective amount of compounds of the present invention. As such, the application may be carried out before or after the infection of the locus, growing crops, or harvested crops by the pest.

The compounds of the present invention can also be applied preventively to places at which occurrence of the pests is expected.

The compounds of the present invention may be also used to protect growing plants from attack or infestation by pests by contacting the plant with a pesticidally effective amount of compounds of the present invention. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 5 g to 500 g per hectare, more desirably from 5 g to 200 g per hectare.

The compounds of the present invention are effective through both contact (via soil, glass, wall, bed net, carpet, plant parts or animal parts), and ingestion (bait, or plant part).

The compounds of the present invention may also be applied against non-crop insect pests, such as ants, termites, wasps, flies, mosquitoes, crickets, or cockroaches. For use against said non-crop pests, compounds of the present invention are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickiness, moisture retention or aging characteristics.

The bait employed in the composition is a product, which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active ingredient.

Formulations of compounds of the present invention as aerosols (e.g in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of the present invention and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of the present invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of the present invention are also suitable for the treatment of plant propagation material, especially seeds, in order to protect them from insect pest, in particular from soil-living insect pests and the resulting plant's roots and shoots against soil pests and foliar insects.

The compounds of the present invention are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the present invention, including a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A 242 236, EP-A 242 246) (WO 92/00377) (EP-A 257 993, U.S. Pat. No. 5,013,659) or in transgenic crop plants, for example cotton, with the capability of producing Bacillus thuringiensis toxins (Bt toxins) which make the plants resistant to certain pests (EP-A 142 924, EP-A 193 259).

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of the present invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

In the treatment of seed, the application rates of the compounds of the present invention are generally from 0.01 g to 10 kg per 100 kg of seed, preferably from 0.05 g to 5 kg per 100 kg of seed, more preferably from 0.1 g to 1000 g per 100 kg of seed and in particular from 0.1 g to 200 g per 100 kg of seed.

The invention therefore also relates to seed comprising a compound of the present invention, including an agriculturally useful salt of it, as defined herein. The amount of the compound of the present invention, including an agriculturally useful salt thereof will in general vary from 0.01 g to 10 kg per 100 kg of seed, preferably from 0.05 g to 5 kg per 100 kg of seed, in particular from 0.1 g to 1000 g per 100 kg of seed. For specific crops such as lettuce the rate can be higher.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of the present invention into contact with each other, and “seed dressing” to methods of seed treatment which provide the seeds with an amount of the compounds of the present invention, i.e. which generate a seed comprising a compound of the present invention. In principle, the treatment can be applied to the seed at any time from the harvest of the seed to the sowing of the seed. The seed can be treated immediately before, or during, the planting of the seed, for example using the “planter's box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of the present invention, including their stereoisomers, veterinarily acceptable salts or N-oxides, are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions comprising a parasiticidally effective amount of compounds of the present invention, including their stereoisomers, veterinarily acceptable salts or N-oxides, and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of the present invention, including its stereoisomers, veterinarily acceptable salts or N-oxides, or a composition comprising it.

The invention also provides a process for the preparation of a composition for treating, controlling, preventing or protecting animals against infestation or infection by parasites which comprises a parasiticidally effective amount of a compound of the present invention, including its stereoisomers, veterinarily acceptable salts or N-oxides, or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula (I) and their stereoisomers, veterinarily acceptable salts, tautomers and N-oxides, are suitable for combating endo- and ectoparasites in and on animals.

The compounds of the present invention, especially compounds of formula (I) and their stereoisomers, veterinarily acceptable salts, tautomers and N-oxides, and compositions comprising them are preferably used for controlling and preventing infestations of and infections in animals including warm-blooded animals (including humans) and fish.

They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of the present invention, including their stereoisomers, veterinarily acceptable salts or N-oxides, and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention, including their stereoisomers, veterinarily acceptable salts or N-oxides, and compositions comprising them are suitable for systemic and/or non-systemic control of ecto- and/or endoparasites. They are active against all or some stages of development.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus, cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
Bugs (Heteropterida): Cimex lectularius, Cimex hemipterus, Reduvius senilis, Triatoma spp., Rhodnius ssp., Panstrongylus ssp. and Arilus critatus,
Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp, Rhabditida, e.g. Rhabditis spp, Strongyloides spp., Helicephalobus spp, Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The present invention relates to the therapeutic and the non-therapeutic use of compounds of the present invention and compositions comprising them for controlling and/or combating parasites in and/or on animals. The compounds of the present invention and compositions comprising them may be used to protect the animals from attack or infestation by parasites by contacting them with a parasiticidally effective amount of compounds of the present invention and compositions containing them.

The compounds of the present invention and compositions comprising them can be effective through both contact (via soil, glass, wall, bed net, carpet, blankets or animal parts) and ingestion (e.g. baits). As such, “contacting” includes both direct contact (applying the pesticidal mixtures/compositions containing the compounds of the present invention directly on the parasite, which may include an indirect contact at its locus-P, and optionally also administrating the pesticidal mixtures/composition directly on the animal to be protected) and indirect contact (applying the compounds/compositions to the locus of the parasite). The contact of the parasite through application to its locus is an example of a non-therapeutic use of compounds of the present invention. “Locus-P” as used above means the habitat, food supply, breeding ground, area, material or environment in which a parasite is growing or may grow outside of the animal.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions of the present invention. A parasiticidally effective amount of the compositions will also vary according to the prevailing conditions such as desired parasiticidal effect and duration, target species, mode of application, and the like.

The compounds of the present invention can also be applied preventively to places at which occurrence of the pests or parasites are expected.

Administration can be carried out both prophylactically and therapeutically.

Administration of the active compounds is carried out directly or in the form of suitable preparations, orally, topically/dermally or parenterally.

### EXAMPLES

The present invention is now illustrated in further details by the following examples, without imposing any limitation thereto.

### Preparation Examples

Compounds can be characterized e.g. by coupled High Performance Liquid Chromatography/mass spectrometry (HPLC/MS), by ¹H-NMR and/or by their melting points.

Analytical HPLC column: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C. ¹H-NMR, respectively ¹³C-NMR: The signals are characterized by chemical shift (ppm, 6 [delta]) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplett, sept=septet, q=quartett, t=triplett, d=doublet, dd=doublet of doublet, s=singulett, b=broad.

Abbreviations used are: h for hour(s), min for minute(s), room temperature for 20-25° C., THF for tetrahydrofuran, n-BuLi for n-butyl-lithium, MTBE for tert-butyl methyl ether.

### C. Compound Examples

### C.1 Compound Examples 1

Compound example 1-1 corresponds to a compound formula C.1:

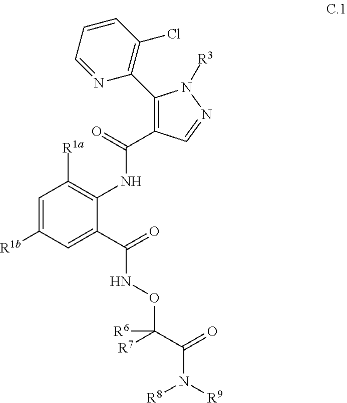

wherein R^{1a}, R^{1b}, R⁶, R⁷, R⁸ and R⁹ are defined in Table C.1 below.

TABLE C.1

| Compound No. | R^{1a} | R^{1b} | R³ | R⁶ | R⁷ | R⁸ | R⁹ |
|---|---|---|---|---|---|---|---|
| 1-1 | CH₃ | Cl | CH₂CF₃ | H | H | CH₃ | H |

TABLE C.2
Physico-chemical data of the compound
example given above in Table C.1

| | Physico-chemical data as melting point mp [° C.] |
|---|---|
| Compound | or retention time tᵣ [min], m/z (HPLC/MS) or |
| No. | ¹H-NMR (CDCl₃) [δ ppm] |
| 1-1 | tᵣ = 2.77 min; m/z = 559 (M⁺) |

### S. Synthesis Examples for Compounds of Formula I

### S.1 Synthesis example of 5-(3-chloro-pyridin-2-yl)-1-(2,2,2-trifluoro-ethyl)-1H-pyrazole-4-carboxylic acid (4-chloro-2-methyl-6-methylcarbamoylmethoxycarbamoyl-phenyl)-amide (compound 1-1 of Table C.1)

### Step 1.1: Synthesis of 3-chloro-pyridine-2-carbonyl chloride

To a solution of 3-chloropicolinic acid (20.0 g, 127 mmol) in toluene (200 mL) was added thionyl chloride (37.7 g, 317 mmol) and dimethylformamide (10 drops). The mixture was heated under reflux (110° C.) for 1 h and evaporated. The residue was taken up into more toluene and evaporated again to give the title product (24.3 g) used in its crude form in the next step.

### Step 1.2: Synthesis of 3-(3-chloro-pyridin-2-yl)-3-oxo-propionic acid ethyl ester

Triethylamine (25.7 g, 254 mmol) and anhydrous magnesium chloride (29.0 g, 305 mmol) were added sequentially to a solution of potassium malonate (43.2 g, 254 mmol) in MeCN (170 mL) at 10-15° C. The mixture was allowed to warm to room temperature and stirred for 3 h, then cooled to 0° C. and a solution of 3-chloro-pyridine-2-carbonyl chloride (24.4 g, 139 mmol) in acetonitrile (50 mL) added slowly, maintaining the temperature between 0-5° C. The mixture was allowed to warm slowly to 25° C. overnight. The mixture was evaporated and the residue was taken up in CH₂CO₂, and 10% HCl was added at 10-15° C. until the aqueous layer reached pH 3. The separated organic phase was washed with 10% HCl, saturated aqueous Na₂CO₃ and again with 10% HCl, dried (Na₂SO₄) and evaporated to give the title product (22.19 g, 70%); LCMS r.t. 2.79, m/z 183 (M-EtOH)+; ¹H NMR (400 MHz, CDCl₃) δ 8.70 (dd, J=1.5, 4.5 Hz, 1H), 7.98 (dd, J=1.5, 8.5 Hz, 1H), 7.56 (dd, J=4.5, 8.5 Hz, 1H), 4.33 (q, J=7.0 Hz, 2H), 4.30 (s, 2H), 1.37 (t, J=7.0 Hz, 3H).

### Step 1.3: Synthesis of (E/Z)-2-(3-chloro-pyridine-2-carbonyl)-3-dimethylamino-acrylic acid ethyl ester

3-(3-Chloro-pyridin-2-yl)-3-oxo-propionic acid ethyl ester (3.0 g, 13.1 mmol) and N,N-dimethylformamide dimethyl acetal (1.94 g, 15.8 mmol) were heated under reflux for 2 h. The mixture was partitioned between ethyl acetate and H₂O. The separated aqueous phase was extracted twice with ethyl acetate and the combined organic phases washed with H₂O twice, dried (Na₂SO₄) and evaporated to give the title product (2.59 g, 70%); LCMS r.t. 2.085; m/z 283 (M+H)+.

### Step 1.4: Synthesis of 5-(3-chloro-pyridin-2-yl)-1-(2,2,2-trifluoro-ethyl)-1H-pyrazole-4-carboxylic acid ethyl ester

To a solution of (E/Z)-2-(3-chloro-pyridine-2-carbonyl)-3-dimethylamino-acrylic acid ethyl ester (2.0 g, 7.1 mmol) in ethanol (34 mL) was added 2,2,2-trifluoroethylhydrazine trihydrate (1.31 g, 7.8 mmol) and the mixture heated to reflux for 2 h. The solvent was evaporated and the residue partitioned between CH₂Cl₂ and H₂O. The aqueous phase was extracted twice with CH₂Cl₂ and the combined organic phases were washed with H₂O, dried (Na₂SO₄) and evaporated to give the title product (1.95 g, 83%); LCMS r.t. 3.16 min, m/z 334.0 (M+H)+. ¹H NMR (400 MHz, CDCl₃) δ 8.64 (dd, J=1.5, 5.0 Hz, 1H), 8.10 (s, 1H), 7.88 (dd, J=1.5, 8.5 Hz, 1H), 7.41 (dd, J=5.0, 8.5 Hz, 1H), 4.90-4.65 (bd m, 2H), 4.24-4.14 (m, 2H), 1.16 (t, J=7.5 Hz, 3H).

### Step 1.5: Synthesis of 5-(3-chloro-pyridin-2-yl)-1-(2,2,2-trifluoro-ethyl)-1H-pyrazole-4-carboxylic acid

To a solution of 5-(3-chloro-pyridin-2-yl)-1-(2,2,2-trifluoro-ethyl)-1H-pyrazole-4-carboxylic acid ethyl ester (1.95 g, 5.8 mmol) in tetrahydrofuran (30 mL) was added a solution of LiOH (0.56 g, 23 mmol) in H₂O (6 mL) and the mixture stirred at 100° C. for 20 h. The mixture was allowed to cool to 25° C. and partitioned between CH₂Cl₂ and 10% hydrochloric acid. The phases were separated and the aqueous phase extracted a further two times. The combined organic phases were washed with H₂O, dried (Na₂SO₄) and evaporated to give the title product (1.50 g, 84%); LCMS r.t. 2.36 min; m/z 307 (M+H)+.

### Step 1.6: Synthesis of 6-chloro-2-[5-(3-chloro-pyridin-2-yl)-1-(2,2,2-trifluoro-ethyl)-1H-pyrazol-4-yl]-8-methyl-benzo[d][1,3]oxazin-4-one

A solution of methanesulfonyl chloride (1.23 g, 10.8 mmol) in acetonitrile (20 mL) was added dropwise at 0° C. to a solution of 5-(3-chloro-pyridin-2-yl)-1-(2,2,2-trifluoro-ethyl)-1H-pyrazole-4-carboxylic acid (3.15 g, 10.3 mmol) and triethylamine (1.50 mL, 10.8 mmol) in acetonitrile (56 mL) and the resulting mixture was stirred for 30 min at 0° C. To this was added 2-amino-5-chloro-3-methylbenzoic acid (1.91 g, 10.3 mmol) and the mixture stirred for 5 min prior to the dropwise addition of a solution of triethylamine (2.79 mL, 20.1 mmol) in acetonitrile (46 mL) and stirring at 0° C. for 1 h. A further portion of methanesulfonyl chloride (1.23 g, 10.8 mmol) was added and the mixture stirred overnight at 25° C. A solution of 5% K₂CO₃ (105 mL) was added and the precipitated solid filtered and dried to yield 1.05 g of crude product. The biphasic filtrate was separated and the upper organic layer was shaken again with 5% aqueous K₂CO₃ (50 mL). A second crop of product was filtered and the two crops combined to give the title product (1.25 g, 27%); LCMS r.t. 4.10 min; m/z 454.9 (M)+; ¹H NMR (400 MHz, DMSO-d₆) δ 8.77 (dd, J=1.5, 5.0 Hz, 1H), 8.38 (s, 1H), 8.21 (dd, J=1.5, 8.0 Hz, 1H), 7.85 (m, 1H), 7.75 (m, 1H), 7.69 (dd, J=4.5, 8.0 Hz, 1H), 5.27-5.00 (m, 2H), 1.93 (s, 3H).

### Step 1.7: Synthesis of 5-(3-Chloro-pyridin-2-yl)-1-(2,2,2-trifluoro-ethyl)-1H-pyrazole-4-carboxylic acid (4-chloro-2-methyl-6-methylcarbamoylmethoxycarbamoyl-phenyl)-amide

A solution of 6-chloro-2-[5-(3-chloro-pyridin-2-yl)-1-(2,2,2-trifluoro-ethyl)-1H-pyrazol-4-yl]-8-methyl-benzo[d][1,3]oxazin-4-one (0.40 g, 0.88 mmol) and 2-aminooxy-NV-methyl-acetamide (0.36 g, 3.5 mmol) in dimethylsulfoxide (10 mL) was heated at 75° C. (bath temperature) for 4 d. The mixture was poured into dilute ice-cold NH₄Cl (20 mL) and the precipitated solids filtered under suction. The filtrate was extracted with CH₂Cl₂ (2×30 mL), dried (Na₂SO₄) and evaporated. The residue was chromatographed and purified further by reverse phase HPLC to give the title compound (72 mg, 15%) as a white solid; LCMS r.t. 2.77 min; m/z 559.1 (M)+; ¹H NMR (400 MHz, DMSO-d₆) δ 11.85 (br s, 1H), 9.86 (br s, 1H), 8.65 (d, J=4.5 Hz, 1H), 8.34 (s, 1H), 8.14 (br s, 1H), 8.05 (d, J=8.5 Hz, 1H), 7.56-7.52 (m, 2H), 7.34 (s, 1H), 5.20-4.96 (br m, 2H), 4.21 (s, 2H), 2.56 (d, J=4.5 Hz, 3H), 2.19 (s, 3H).

### B. Biological Examples

The activity of the compounds of formula I of the present invention could be demonstrated and evaluated in biological tests described in the following.

The active compound was dissolved at the desired concentration in a mixture of 1:1 (vol:vol) distilled water: acteon. The test solution was prepared at the day of use.

### B.1 Diamond Backmoth (Plutella xylostella)

The active compound was dissolved at the desired concentration in a mixture of 1:1 (vol:vol) distilled water: aceteone. Surfactant (Alkamuls® EL 620) was added at a rate of 0.1% (vol/vol). The test solution was prepared at the day of use.

Leaves of cabbage were dipped in test solution and air-dried. Treated leaves were placed in petri dishes lined with moist filter paper and inoculated with ten 3^{rd} instar larvae. Mortality was recorded 72 hours after treatment. Feeding damages were also recorded using a scale of 0-100%.

In this test, compound 1-1 at 500 ppm showed 100% mortality in comparison with untreated controls.

### B.2 Southern Armyworm (Spodoptera eridiana); 2^{nd} Instar Larvae

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic) was included in the solution at a volume of 0.01% (v/v).

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, compound 1-1 at 500 ppm showed 100% mortality in comparison with untreated controls.

1-32. (canceled)

33. A compound of formula (I) 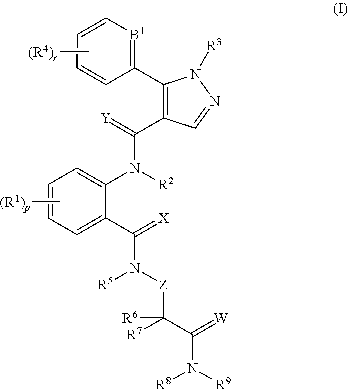
wherein
B¹ is N or CH;
each R¹ is independently selected from the group consisting of halogen; cyano; azido; nitro; —SCN; SF₅; C₁-C₆-alkyl which may be partially or fully halogenated and/or may be substituted by one or more radicals R¹⁰; C₃-C₈-cycloalkyl which may be partially or fully halogenated and/or may be substituted by one or more radicals R¹⁰; C₂-C₆-alkenyl which may be partially or fully halogenated and/or may be substituted by one or more radicals R¹⁰; C₂-C₆-alkynyl which may be partially or fully halogenated and/or may be substituted by one or more radicals R¹⁰; —Si(R¹⁴)₂R¹⁵; —OR¹¹; —OS(O)ₙR¹¹;
—SR¹¹; —S(O)ₘR¹¹; —S(O)ₙN(R^{12a})R^{12b}; —N(R^{12a})R^{12b}; —N(R^{12a})C(═O)R¹⁰; —C(═O)R¹⁰; —C(═S)R¹⁰; —C(═O)OR¹¹; —C(═S)OR¹¹; —C(═NR^{12a})R¹⁰; —C(═O)N(R^{12a})R^{12b}; —C(═S)N(R^{12a})R^{12b}; phenyl which may be substituted by 1, 2, 3, 4 or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
or two radicals R¹ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²¹—, —CH₂CH═N—, —CH═CH—NR²¹—, —OCH═N— and —SCH═N—, thus forming, together with the carbon atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R² is selected from the group consisting of hydrogen; cyano; C₁-C₁₀-alkyl which may be partially or fully halogenated and/or may be substituted by one or more radicals R¹⁰; C₃-C₈-cycloalkyl which may be partially or fully halogenated and/or may be substituted by one or more radicals R¹⁰; C₂-C₁₀-alkenyl which may be partially or fully halogenated and/or may be substituted by one or more radicals R¹⁰; C₂-C₁₀-alkynyl which may be partially or fully halogenated and/or may be substituted by one or more radicals R¹⁰;
—N(R^{12a})R^{12b}; —Si(R¹⁴)₂R¹⁵; —OR¹¹; —SR¹¹; —S(O)ₘR¹¹; —S(O)ₙN(R^{12a})R^{12b}, —C(═O)R¹⁰; —C(═O)OR¹¹; —C(═O)N(R^{12a})R^{12b}; —C(═S)R¹⁰; —C(═S)OR¹¹; —C(═S)N(R^{12a})R^{12b}; —C(═NR^{12a})R¹⁰; phenyl which may be substituted by 1, 2, 3, 4 or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
R³ is selected from the group consisting of hydrogen, cyano, nitro, C₁-C₆-alkyl which may be partially or fully halogenated and/or may be substituted by one or more radicals R⁷, C₃-C₈-cycloalkyl which may be partially or fully halogenated and/or may be substituted by one or more radicals R⁷, C₂-C₆-alkenyl which may be partially or fully halogenated and/or may be substituted by one or more radicals R⁷, C₂-C₆-alkynyl which may be partially or fully halogenated and/or may be substituted by one or more radicals R⁷, —Si(R¹⁴)₂R¹³, —OR⁸, —OS(O)ₙR⁸, —SR⁸, —S(O)ₘR⁸, —S(O)ₙN(R^{9a})R^{9b}, —N(R^{9a})R^{9b}, N(R^{9a})C(═O)R⁷, —C(═O)R⁷, —C(═O)OR⁸, —C(═S)R⁷, —C(═S)OR⁸, —C(═NR^{9a})R⁷, —C(═O)N(R^{9a}) R^{9b}, —C(═S)N(R^{9a})R^{9b}, phenyl which may be substituted by 1, 2, 3, 4 or 5 radicals R¹⁰, and a 3-, 4-, 5-, 6- or 7-membered saturated, partially unsaturated or maximum unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R⁴ is independently selected from the group consisting of halogen, cyano, azido, nitro, —SCN, SF₅, C₁-C₆-alkyl which may be partially or fully halogenated and/or may be substituted by one or more radicals R¹⁰, C₃-C₈-cycloalkyl which may be partially or fully halogenated and/or may be substituted by one or more radicals R¹⁰, C₂-C₆-alkenyl which may be partially or fully halogenated and/or may be substituted by one or more radicals R , C₂-C₆-alkynyl which may be partially or fully halogenated and/or may be substituted by one or more radicals R¹⁰, —Si(R¹⁴)₂R¹⁵, —OR¹, —SR¹¹,
—S(O)ₘR¹¹, S(O)ₙN(R^{12a})R^{12b}, —N(R^{12a})R^{12b}, —N(R^{12a})C(═O)R¹⁰, —C(═O)R¹⁰, —C(═O)OR¹¹, —C(═S)R¹⁰, —C(═S)OR¹¹, —C(═NR^{12a})R¹⁰, —C(═O)N(R^{12a})R^{12b}, —C(═S)N(R^{12a})R^{12b}, phenyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
R⁵ is selected from the group consisting of hydrogen; cyano; C₁-C₁₀-alkyl which may be partially or fully halogenated and/or may be substituted by one or more radicals R¹⁰; C₃-C₈-cycloalkyl which may be partially or fully halogenated and/or may be substituted by one or more radicals R¹⁰; C₂-C₁₀-alkenyl which may be partially or fully halogenated and/or may be substituted by one or more radicals R¹⁰; C₂-C₁₀-alkynyl which may be partially or fully halogenated and/or may be substituted by one or more radicals R¹⁰;
—N(R^{12a})R^{12b}; —Si(R¹⁴)₂R¹⁵; —OR¹¹; —SR¹¹; —S(O)ₘR¹¹; —S(O)ₙN(R^{12a})R^{12b}, —C(═O)R¹⁰; —C(═O)OR¹¹; —C(═O)N(R^{12a})R^{12b}; —C(═S)R; —C(═S)OR¹¹; —C(═S)N(R^{12a})R^{12b}; —C(═NR^{12a})R¹⁰; phenyl which may be substituted by 1, 2, 3, 4 or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
R⁶, R⁷ are selected independently of each other from the group consisting of hydrogen, halogen, cyano, azido, nitro, —SCN, C₁-C₆-alkyl, C₃-C₆-cycloalkyl, C₂-C₆-alkenyl, C₂-C₆-alkynyl, wherein the aforementioned aliphatic and cycloaliphatic radicals each independently may be partially or fully halogenated and/or may be substituted with 1, 2, 3, 4, 5, 6, 7, 8, 9 or 10 substituents R¹⁰, said substituents R¹⁰ being identical or different from one another if more than one substituent R¹⁰ is present, —OR¹¹, —NR^{12a}R^{12b},
—S(O)ₙR¹¹, —S(O)ₙNR^{12a}R^{12b}, —C(═O)R¹⁰, —C(═O)NR^{12a}R^{12b}, —C(═O)OR¹¹, —C(═S)R¹⁰, —C(═S)NR^{12a}R^{12b}, —C(═S)OR¹¹, —C(═S)SR¹¹, —C(═NR^{12a})R¹⁰, —C(═NR^{12a})NR^{12a}R^{12b}, —Si(R¹⁴)₂R¹⁵, phenyl which may be substituted with 1, 2, 3, 4 or 5 substituents R¹³, said substituents R¹³ being identical or different from one another if more than one substituent R¹³ is present, and a 3-, 4-, 5-, 6- or 7-membered saturated, partially unsaturated or maximally unsaturated heterocyclic ring wherein said heterocyclic ring comprises 1, 2 or 3 heteroatoms independently selected from the group consisting of oxygen, nitrogen and sulfur atoms and may be substituted with 1, 2, 3, 4 or 5 substituents R¹³, said substituents R¹³ being identical or different from one another if more than one substituent R¹³ is present, and wherein said nitrogen and sulfur atoms, independently of one another, may be oxidized;
or R⁶ and R⁷ are together a C₂-C₇-alkylene or C₂-C₇-alkenylene chain and form a 3-, 4-, 5-, 6-, 7- or 8-membered saturated, partially unsaturated or maximally unsaturated ring together with the carbon atom they are bonded to, wherein 1, 2, 3 or 4 of any of the CH₂ groups in the C₂-C₇-alkylene chain or 1, 2, 3 or 4 of any of the CH₂ or CH groups in the C₂-C₇-alkenylene chain may be replaced by 1, 2, 3 or 4 groups independently selected from the group consisting of O, S, N and NH; and wherein the carbon and/or nitrogen atoms in the C₂-C₇-alkylene or C₂-C₇-alkenylene chain may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl and phenyl which may be substituted with 1, 2, 3, 4 or 5 substituents R¹³, said substituents R¹³ being identical or different from one another if more than one substituent R¹³ is present; and wherein the sulfur and nitrogen atoms in the C₂-C₇-alkylene, C₂-C₇-alkenylene or C₂-C₇-alkynylene chain, independently of one another, may be oxidized;
or R⁶ and R⁷ together form a ═O, ═S, ═NR^{12a}, ═NOR¹¹, or ═CR¹⁶R¹⁷ radical;
R⁸, R⁹ are selected independently of each other from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals each independently may be partially or fully halogenated and/or may be substituted with 1, 2, 3, 4, 5, 6, 7, 8,9 or 10 substituents R¹⁰, said substituents R¹⁰ being identical or different from one another if more than one substituent R¹⁰ is present, —OR¹¹, —NR^{12a}R^{12b}, —S(O)ₙR¹¹, —S(O)ₙNR^{12a}R^{12b},
—C(═O)R¹⁰, —C(═O)NR^{12a}R^{12b}, —C(═O)OR¹¹, —C(═S)R¹³, —C(═S)NR^{12a}R^{12b}, —C(═S)OR¹¹, —C(═S)SR¹¹, —C(═NR^{12a})R¹³, —C(═NR^{12a})NR^{12a}R^{12b}, —Si(R¹⁴)₂R¹⁵, phenyl which may be substituted with 1, 2, 3, 4 or 5 substituents R¹³, said substituents R¹³ being identical or different from one another if more than one substituent R¹³ is present, and a 3-, 4-, 5-, 6- or 7-membered saturated, partially unsaturated or maximum unsaturated heterocyclic ring wherein said heterocyclic ring comprises 1, 2 or 3 heteroatoms independently selected from the group consisting of oxygen, nitrogen and sulfur atoms and may be substituted with 1, 2, 3, 4 or 5 substituents R²¹, said substituents R²¹ being identical or different from one another if more than one substituent R²¹ is present, and wherein said nitrogen and sulfur atoms, independently of one another, may be oxidized;
or R⁸ and R⁹, together with the nitrogen atom to which they are bound, form a 3-, 4-, 5-, 6-, 7- or 8-membered saturated, partially unsaturated or maximally unsaturated heterocyclic ring which may additionally contain 1, 2, 3 or 4 further heteroatoms or heteroatom groups selected from N, O, S, NO, SO, SO₂, C(═O) and C(═S) as ring members, where the heterocyclic ring may be substituted by one or more radicals R¹³;
or R⁸ and R⁸ together may form a═CR¹⁶R¹⁷, ═S(O)ₙR¹¹, ═S(O)ₙNR^{12a}R^{12b}, ═NR^{12a} or ═NOR¹¹ radical;
each R¹⁰ is independently selected from the group consisting of cyano, azido, nitro, —SCN, SF₅, C₃-C₈-cycloalkyl, C₃-C₈-halocycloalkyl, —Si(R¹⁴)₂R¹⁵, —OR¹¹, —OSO₂R¹¹, —SR¹¹, —S(O)ₘR¹¹, —S(O)ₙN(R^{12a})R^{12b}, —N(R^{12a})R^{12b}, —C(═O)N(R^{12a})R^{12b}, —C(═S)N(R^{12a})R^{12b}, —C(═O)OR¹¹, —C(═O)R²⁰, phenyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
and, in case R¹⁰ is bound to a cycloalkyl group or to a heterocyclic ring, R¹⁰ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹³;
and in groups —C(═O)R¹⁰, —C(═S)R¹⁰, —C(═NR^{12a})R¹⁰ and —N(R^{12a})C(═O)R¹⁰, R¹⁰ may additionally be selected from hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl which may be substituted by 1, 2, 3, 4 or 5 radicals R¹³;
or two geminally bound radicals R¹⁰ together form a group selected from ═CR¹⁶R¹⁷, ═S(O)ₘR¹¹, ═S(O)ₘN(R^{12a})R^{12b}, ═NR^{12a}, ═NOR¹¹ and ═NN(R^{12a})R^{12b};
or two radicals R¹⁰, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R¹¹ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹⁵, —SR²³, —S(O)ₘR²³, —S(O)ₙN(R^{12a})R^{12b}, —N(R^{12a})R^{12b}, —N═CR¹⁸R¹⁹, —C(═O)R²⁰, —C(═O)N(R^{12a})R^{12b}, —C(═S)N(R^{12a})R^{12b}, —C(═O)OR²³, phenyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
with the proviso that R¹¹ is not C₁-C₆-alkoxy or C₁-C₆-haloalkoxy if it is bound to an oxygen atom;
R^{12a}, R^{12b} are, independently of each other and independently of each occurrence, selected from the group consisting of hydrogen, cyano, C₁-C₆-alkyl which may be partially or fully halogenated and/or may be substituted by one or more radicals R²², C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R²², C₃-C₈-cycloalkyl which may be partially or fully halogenated and/or may be substituted by one or more radicals R²², C₃-C₈-cycloalkyl-C₁-C₄-alkyl where the cycloalkyl moiety may be partially or fully halogenated and/or may be substituted by one or more radicals R²², C₂-C₆-alkenyl which may be partially or fully halogenated and/or may be substituted by one or more radicals R²², C₂-C₆-alkynyl which may be partially or fully halogenated and/or may be substituted by one or more radicals R²², —N(R²⁴)R²⁵; —N(R²⁴)C(═O)R²⁰; —Si(R¹⁴)₂R¹⁵; —OR²³; SR²³;
—S(O)ₘR²³; —S(O)ₙN(R²⁴)R²⁵; —C(═O)R²⁰; —C(═O)OR²³; —C(═O)N(R²⁴)R²⁵; —C(═S)R²⁰; —C(═S)OR²³; —C(═S)N(R²⁴)R²⁵; —C(═NR²⁴)R²⁰; —S(O)ₘR²³, —S(O)ₙN(R²⁴)R²⁵; phenyl which may be substituted by 1, 2, 3, 4 or 5 radicals R¹³; benzyl which may be substituted by 1, 2, 3, 4 or 5 radicals R¹³, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹³;
or R^{12a} and R^{12b} together form a group ═CR¹⁶R¹⁷;
or R^{12a} and R^{12b}, together with the nitrogen atom to which they are bound, may form a 3-, 4-, 5-, 6- or 7-membered saturated, partially unsaturated or maximally unsaturated heterocyclic ring which may additionally containing 1 or 2 further heteroatoms or heteroatom groups selected from N, O, S, NO, SO and SO₂, as ring members, where the heterocyclic ring may be substituted by one or more radicals R¹³;
each R¹³ is independently selected from the group consisting of halogen, cyano, azido, nitro, —SCN, SF₅, C₁-C₁₀-alkyl which may be partially or fully halogenated and/or may be substituted by one or more radicals R²², C₃-C₈-cycloalkyl which may be partially or fully halogenated and/or may be substituted by one or more radicals R²², C₂-C₁₀-alkenyl which may be partially or fully halogenated and/or may be substituted by one or more radicals R²², C₂-C₁₀-alkynyl which may be partially or fully halogenated and/or may be substituted by one or more radicals R²², —Si(R¹⁴)₂R¹⁵, —OR²³, —OS(O)ₙR²³,
—SR²³, —S(O)ₘR²³, —S(O)ₙN(R²⁴)R²⁵, —N(R²⁴)R²⁵, C(═O)R²⁰, —C(═O)OR²³, —C(═NR²⁴)R²⁰, —C(═O)N(R²⁴)R²⁵, —C(═S)N(R²⁴)R²⁵, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;
or two radicals R¹³ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²⁴—, —CH₂CH═N—, —CH═CH—NR²⁴—, —OCH═N— and —SCH═N—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R¹⁴, R¹⁵ are, independently of each other and independently of each occurrence, selected from the group consisting of C₁-C₄-alkyl, C₃-C₆-cycloalkyl, C₁-C₄-alkoxy-C₁-C₄-alkyl, phenyl and benzyl;
R¹⁶, R¹⁷ are, independently of each other and independently of each occurrence, selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, —C(═O)R²⁰, —C(═O)OR²³, —C(═NR²⁴)R²⁰, —C(═O)N(R²⁴)R²⁵, —C(═S)N(R²⁴)R²⁵, phenyl which may be substituted by 1, 2, 3, 4, or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R¹³;
R¹⁸, R¹⁹ are, independently of each other and independently of each occurrence, selected from the group consisting of C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R¹³; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R¹³;
each R²⁰ is independently selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
each R²¹ is independently defined like R²;
each R²² is independently selected from the group consisting of cyano, azido, nitro, —SCN, SF₅, C₃-C₈-cycloalkyl, C₃-C₈-halocycloalkyl, —Si(R¹⁴)₂R¹⁵, —OR²³, —OSO₂R²³, —SR²³, —S(O)ₘR²³, —S(O)ₙN(R²⁴)R²⁵, —N(R²⁴)R²⁵, —C(═O)N(R²⁴)R²⁵, —C(═S)N(R²⁴)R²⁵, —C(═O)OR²³, —C(═O)R²³, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
and, in case R²² is bound to a cycloalkyl group, R²² may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl and C₂-C₆-haloalkynyl;
or two geminally bound radicals R²² together form a group selected from ═S(O)ₘR²³, ═S(O)ₘN(R²⁴)R²⁵, ═NR²⁴, ═NOR²³ and ═NN(R²⁴)R²⁵;
or two radicals R²², together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R²³ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹⁵, C₁-C₆-alkylaminosulfonyl, amino, C₁-C₆-alkylamino, di-(C₁-C₆-alkyl)-amino, C₁-C₆-alkylcarbonyl, C₁-C₆-haloalkylcarbonyl, aminocarbonyl, C₁-C₆-alkylaminocarbonyl, di-(C₁-C₆-alkyl)-aminocarbonyl, C₁-C₆-alkoxycarbonyl, C₁-C₆-haloalkoxycarbonyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
with the proviso that R²³ is not C₁-C₆-alkoxy or C₁-C₆-haloalkoxy if it is bound to an oxygen atom;
R²⁴ and R²⁵ are independently of each other and independently of each occurrence selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
or R²⁴ and R²⁵, together with the nitrogen atom to which they are bound, may form a 3-, 4-, 5-, 6- or 7-membered saturated, partially unsaturated or maximally unsaturated heterocyclic ring which may additionally contain 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy;
each m is independently 1 or 2;
each n is independently 0, 1 or 2;
p is 0, 1,2,3 or 4;
r is 0, 1, 2, 3, or 4;
W is O or S;
X is O or S;
Y is O or S; and
Z is O, NR^{12a} or S(O)ₙ
or a stereoisomer, salt, tautomer or N-oxide thereof.

34. The compound according to claim 33, wherein W, X and Y are O.

35. The compound according to claim 33, wherein
p is 1, 2 or 3, preferably 2.

36. The compound according to claim 33, wherein
r is 0, 1, or 2, preferably 1.

37. The compound according to claim 33, wherein B¹ is N.

38. The compound according to claim 33, wherein each R¹ is independently selected from halogen, cyano and C₁-C₆-alkyl which may be partially or fully halogenated and/or may be substituted by one or more radicals R¹⁰, and preferably from halogen and C₁-C₄-alkyl.

39. The compound according to claim 33, wherein R² is hydrogen or C₁-C₆-alkyl which may be partially or fully halogenated and/or may be substituted by one or more radicals R¹⁰, and is preferably hydrogen.

40. The compound according to claim 33, wherein R³ is selected from hydrogen, cyano and C₁-C₆-alkyl which may be partially or fully halogenated and/or may be substituted by one or more radicals R¹⁰ and is preferably C₁-C₆-alkyl or C₁-C₆-haloalkyl.

41. The compound according to claim 33, where R³ is not hydrogen.

42. The compound according to claim 33, wherein each R⁴ is independently selected from halogen, cyano and C₁-C₆-alkyl which may be partially or fully halogenated and/or may be substituted by one or more radicals R¹⁰, and is preferably halogen.

43. The compound according to claim 33, wherein R⁵ is hydrogen or C₁-C₆-alkyl which may be partially or fully halogenated and/or may be substituted by one or more radicals R¹⁰, and is preferably hydrogen.

44. The compound according to claim 33, wherein R⁶ and R⁷, independently of each other, are selected from hydrogen, halogen and C₁-C₆-alkyl which may be partially or fully halogenated and/or may be substituted by one or more radicals R¹⁰, and are preferably hydrogen.

45. The compound according to claim 33, wherein R⁸ and R⁹, independently of each other, are selected from the group consisting of hydrogen, C₁-C₆-alkyl which may be partially or fully halogenated and/or may be substituted by one or more radicals R¹⁰, —C(═O)R¹⁰, —C(═O)OR¹¹, and —C(═O)N(R^{12a})R^{12b}, or, together with the nitrogen atom to which they are bound, form a 5- or 6-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals selected from halogen, cyano, C₁-C₆-alkyl, C₁-C₆-haloalkyl, C₁-C₆-alkoxy and C₁-C₆-haloalkoxy.

46. The compound according to claim 45, wherein R⁸ and R⁹, independently of each other, are selected from the group consisting of hydrogen, C₁-C₆-alkyl and C₁-C₆-haloalkyl.

47. The compound according to claim 33, wherein Z is O.

48. The compound according to claim 33, wherein Z is NR^{12a}.

49. The compound according to claim 48, wherein R^{12a} is hydrogen or C₁-C₆-alkyl which may be partially or fully halogenated and/or may be substituted by one or more radicals R²², and is preferably hydrogen.

50. The compound according to claim 33, wherein the compound has the general formula (I-a) 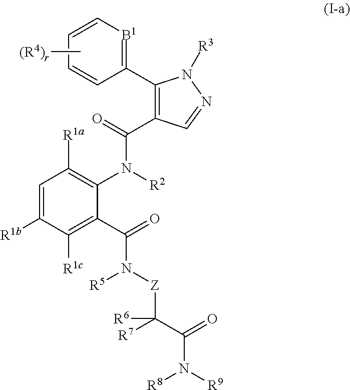
wherein
R^{1a}, R^{1b} and R^{1c} are selected from hydrogen and the group as defined for R¹.

51. The compound according to claim 50, wherein the compound has the general formula (I-aa) 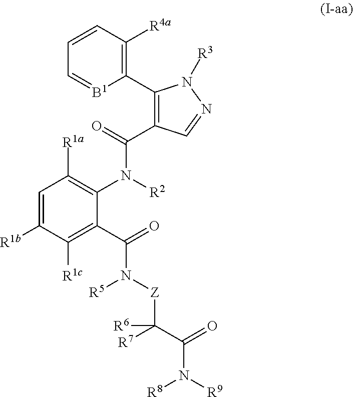
wherein
R^{1a}, R^{1b} and R^{1c} are selected from hydrogen and the group as defined for R¹;
R^{4a} is selected from hydrogen and the group as defined for R⁴.

52. The compound according to claim 51 of the formula (I-aa), wherein B¹ is N, Z is O, R^{1c}, R², R⁵ and R⁶ are H, R^{1a} is CH₃, R^{4a} is Cl, and R^{1b}, R³, R⁷, R⁸ and R⁹ have the following meanings:
R^{1b} is Cl, R³ is CH₂CF₃, R⁷ is H, R⁸ is CH₃ and R⁹ is CH₃; or
R^{1b} is Cl, R³ is CH₂CF₃, R⁷ is H, R⁸ is CH₃ and R⁹ is H; or
R^{1b} is CN, R³ is CH₂CF₃, R⁷ is H, R⁸ is CH₃ and R⁹ is CH₃; or
R^{1b} is CN, R³ is CH₂CF₃, R⁷ is H, R⁸ is CH₃ and R⁹ is H; or
R^{1b} is Cl, R³ is CH₂CF₃, R⁷ is H, R⁸ is CH(CH₃)₂ and R⁹ is H; or
R^{1b} is Cl, R³ is CH₂CF₃, R⁷ is CH₃, R⁸ is CH₃ and R⁹ is CH₃; or
R^{1b} is Cl, R³ is CH₂CF₃, R⁷ is CH₃, R⁸ is CH₃ and R⁹ is H; or
R^{1b} is Cl, R³ is CH₂CHF₂, R⁷ is H, R⁸ is CH₃ and R⁹ is CH₃; or
R^{1b} is Cl, R³ is CH₂CHF₂, R⁷ is H, R⁸ is CH₃ and R⁹ is H; or
R^{1b} is CN, R^{3a} is CH₂CHF₂, R⁷ is H, R⁸ is CH₃ and R⁹ is CH₃; or
R^{1b} is CN, R³ is CH₂CHF₂, R⁷ is H, R⁸ is CH₃ and R⁹ is H; or
R^{1b} is Cl, R³ is CH₂CHF₂, R⁷ is H, R⁸ is CH(CH₃)₂ and R⁹ is H; or
R^{1b} is Cl, R³ is CH₂CHF₂, R⁷ is CH₃, R⁸ is CH₃ and R⁹ is CH₃; or
R^{1b} is Cl, R³ is CH₂CHF₂, R⁷ is CH₃, R⁸ is CH₃ and R⁹ is H.

53. A method for preparing the compound of formula (I) as defined in claim 33, comprising following steps:
reacting a compound of formula (II) or a compound of formula (III) 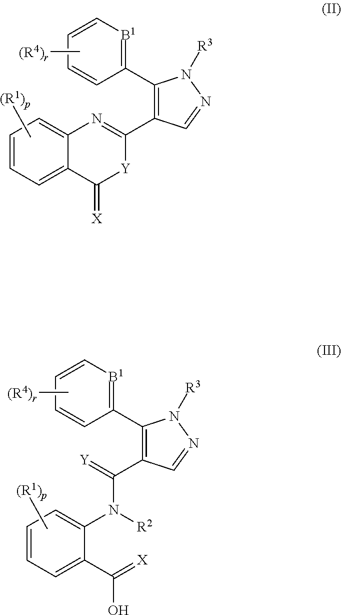
with a compound of formula IV 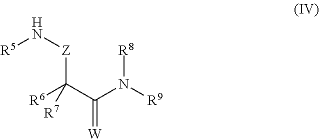
where in case of reaction of compound (11), a compound of formula (I) wherein R² is hydrogen is obtained, and,
if desired, reacting the compound (I) wherein R² is hydrogen with a compound R²—Z′ wherein R² is different from hydrogen and Z′ is a leaving group.

54. An agricultural or veterinary composition comprising at least one compound as defined in claim 33, or a stereoisomer, agriculturally or veterinarily acceptable salt, tautomer or N-oxide thereof and at least one liquid and/or solid carrier.

55. A method for combating or controlling invertebrate pests of the group of insects, arachnids or nematodes, which method comprises contacting said pest or its food supply, habitat or breeding grounds with a pesticidally effective amount of at least one compound as defined in claim 33 or a stereoisomer, salt, tautomer or N-oxide thereof.

56. A method for protecting growing plants from attack or infestation by invertebrate pests of the group of insects, arachnids or nematodes, which method comprises contacting a plant, or soil or water in which the plant is growing or may grow, with a pesticidally effective amount of at least one compound as defined in claim 33 or a stereoisomer, salt, tautomer or N-oxide thereof.

57. A method for the protection of seeds from soil insects and of the seedlings' roots and shoots from soil and foliar insects comprising contacting the seeds before sowing and/or after pregermination with at least one compound as defined in claim 33 or a stereoisomer, salt, tautomer or N-oxide thereof.

58. Seed comprising a compound as defined in claim 33 or a stereoisomer, salt, tautomer or N-oxide thereof in an amount of from 0.1 g to 10 kg per 100 kg of the plant propagation material.

59-61. (canceled)

62. A method for treating an animal infested or infected by parasites or for preventing animals from getting infested or infected by parasites or for protecting an animal against infestation or infection by parasites which comprises orally, topically or parenterally administering or applying to the animal a parasiticidally effective amount of a compound as defined in claim 33 or a stereoisomer, veterinarily acceptable salt, tautomer or N-oxide thereof.

63-64. (canceled)
